SEQUENCE LISTING (15 sequences)

SEQ ID NO: 1 - DNA, 1436 bp, Bacteroides fragilis
atgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaac

SEQ ID NO: 2 - DNA, 1395 bp, Bacteroides ovatus
  modified_base (51)..(51): a, c, t, g, unknown or other
  modified_base (125)..(125): a, c, t, g, unknown or other
  modified_base (154)..(154): a, c, t, g, unknown or other
  modified_base (168)..(168): a, c, t, g, unknown or other
  modified_base (170)..(170): a, c, t, g, unknown or other
  modified_base (177)..(177): a, c, t, g, unknown or other
  modified_base (180)..(180): a, c, t, g, unknown or other
  modified_base (393)..(393): a, c, t, g, unknown or other
  modified_base (435)..(435): a, c, t, g, unknown or other
  modified_base (720)..(721): a, c, t, g, unknown or other
  modified_base (902)..(902): a, c, t, g, unknown or other
  modified_base (973)..(973): a, c, t, g, unknown or other
  modified_base (999)..(999): a, c, t, g, unknown or other
  modified_base (1082)..(1082): a, c, t, g, unknown or other
  modified_base (1232)..(1232): a, c, t, g, unknown or other
  modified_base (1235)..(1235): a, c, t, g, unknown or other
atgaacgctagctacaggcttaacacatgcaagtcgaggggcagcattttngtttgcttgcaaactgaagatggcgaccggcgcacgggtgagtaacacgtatccaacctgccgataactccggnatagcctttcgaaagaaagattaataccngatagcatacgaanancgcatgntanttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatganggccctatgggtcgtaaacttcttttatatgggaataaagtnttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgnnactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggccngcacaagcggaggaacatgtggtttaattcgatgatacgcgaggaaccttacccgggcttaaattgcaacngaatatattggaaacagtatagccgnaaggctgttgtgaaggtgctgcatggttgtcgtcagctcgtgccgtgaggtgtcggcttaagtgccataacgagcgcaacccntatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctaccnggngacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggt

SEQ ID NO: 3 - DNA, 1510 bp, Bacteroides vulgatus
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaag

SEQ ID NO: 4 - DNA, 1467 bp, Bilophila wadsworthia
  modified_base (789)..(789): a, c, t, g, unknown or other
cttaacacatgcaagtcgaacgtgaaagtccttcgggatgagtaaaagtggcgcacgggtgagtaacgcgtggataatctacccttaagatggggataacggctggaaacggtcgctaataccgaatacgctcccgattttatcattggggggaaagatggcctctgcttgcaagctatcgcttaaggatgagtccgcgtcccattagctagttggcggggtaacggcccaccaaggcaacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtacgcggcttggtaagtcaggggtgaaatcccacagcccaactgtggaactgcctttgatactgccacgcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgcngggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactcgcaagaggagcagccgtctacggtagggccgatgattggggtgaagtcgtaacaa

SEQ ID NO: 5 - DNA, 1476 bp, Clostridium bifermentans
  modified_base (1449)..(1449): a, c, t, g, unknown or other
catrgctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatatgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatatgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcactaacgcattaagtactccgcctgggaagtacgctcgcaagagtgaaactcaaaggaatttdcggggacccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaacaaatgactggggtgaagtcgtaacaaggtanccgtatcggaaggtgcggcbggatcaa

SEQ ID NO: 6 - DNA, 1393 bp, Clostridium hiranonis
acatgcaagtcgagcgattctcttcggagaagagcggcggacgggtgagtaacgcgtgggtaacctgccctgtacacacggataacataccgaaaggtatgctaatacgggataatatataagagtcgcatgacttttatatcaaagatttttcggtacaggatggacccgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagttaaaggctacggctcaaccgtagtaagctcctgatactgtctgacttgagtgcaggagaggaaagcggaattcccagtgtagcggtgaaatgcgtagatattgggaggaacaccagtagcgaaggcggctttctggactgtaactgacgctgaggcacgaaagcgtggggagcaaacaggattagataccctggtagtccacgctgtaaacgatgagtactagttgtcggaggttaccccttcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaggcttgacatccttctgaccgaggactaatctcctctttccctccggggacagaagtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcctagggctacacacgtgctacaatgggtggtacagagggcagccaagccgtgaggtggagcaaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttggagacacccgaagccgactatctaaccttttgggagaagtcgtccccctcgaatcaatacccc

SEQ ID NO: 7 - DNA, 1465 bp, Clostridium leptum
  modified_base (1)..(84): a, c, t, g, unknown or other
  modified_base (233)..(233): a, c, t, g, unknown or other
  modified_base (271)..(271): a, c, t, g, unknown or other
  modified_base (337)..(338): a, c, t, g, unknown or other
  modified_base (389)..(389): a, c, t, g, unknown or other
  modified_base (482)..(485): a, c, t, g, unknown or other
  modified_base (523)..(523): a, c, t, g, unknown or other
  modified_base (659)..(659): a, c, t, g, unknown or other
  modified_base (670)..(670): a, c, t, g, unknown or other
  modified_base (812)..(813): a, c, t, g, unknown or other
  modified_base (822)..(822): a, c, t, g, unknown or other
  modified_base (827)..(828): a, c, t, g, unknown or other
  modified_base (906)..(908): a, c, t, g, unknown or other
  modified_base (932)..(932): a, c, t, g, unknown or other
  modified_base (945)..(946): a, c, t, g, unknown or other
  modified_base (968)..(968): a, c, t, g, unknown or other
  modified_base (1017)..(1017): a, c, t, g, unknown or other
  modified_base (1079)..(1080): a, c, t, g, unknown or other
  modified_base (1095)..(1095): a, c, t, g, unknown or other
  modified_base (1186)..(1187): a, c, t, g, unknown or other
  modified_base (1224)..(1224): a, c, t, g, unknown or other
  modified_base (1365)..(1366): a, c, t, g, unknown or other
  modified_base (1461)..(1462): a, c, t, g, unknown or other
nnnnnnnnnnnnnnnnnnnnnnnnnnnnnnnnnnnnnnnnnnnnnnnnnnnnnnnnnnnnnnnnnnnnnnnnnnnnnnnnnnnnttggatttaacttagtggcggacgggtgagtaacgcgtgagtaacctgcctttcagagggggataacgttctgaaaagaacgctaataccgcataacatcaatttatcgcatgataggttgatcaaaggagcaatccgctggaagatgnactcgcgtccgattagccagttggcggggtaacggccnaccaaagcgacgatcggtagccggactgagaggttgaacggccacattgggactgagacacggccnngactcctacgggaggcagcagtgggggatattgcacaatgggggaaacccngatgcagcaacgccgcgtgagggaagaaggttttcggattgtaaacctctgttcttagtgacgataatgacggtagctaaggagaaagctccnnnnaactacgtgccagcagccgcggtaatacgtagggagcnagcgttgtccggatttactgggtgtaaagggtgcgtaggcggcgaggcaagtcaggcgtgaaatctatgggcttaacccataaactgcgcttgaaactgtcttgcttgagtgaagtagaggtaggcggaattcccngtgtagcggtnaaatgcgtagagatcgggaggaacaccagtggcgaaggcggcctactgggctttaactgacgctgaagcacgaaagcatgggtagcaaacaggattagataccctggtagtccatgccgtaaacgatgattactaggtgtgnngggggtctnacccnntccgtgccgcagttaacacaataagtaatccacctggggagtacggccgcaaggttgaaactcaaaggaattgacggnnncccgcacaagcagtggagtatgtngtttaattcgaannaacgcgaagaaccttaccaggncttgacatccgtctaacgaagcagagatgcattaggtgcccttcggggnaaggcgagacaggtggtgcatggttgtcgtcagctcgtgtcgtgagatgttgggttaagtcnngcaacgagcgcaacncttgtttctagttgctacgcaagagcactctagagagactgccgttgacaaaacggaggaaggtggggacgacgtcaaatcatcatgcccnntatgacctgggccacacacgtactacaatggctgtanacagagggaagcaaagccgcgaggtggagcaaaaccctaaaagcagtcccagttcggatcgcaggctgcaacccgcctgcgtgaagtcggaattgctagtaatcgcggatcagcatgccgcggtgaatacgttcccgggcnntgtacacaccgcccgtcacaccatgggagccggtaatacccgaagccagtagttcaaccgcaaggagagcgctgtcgaaggtaggattggcgacnnggg

SEQ ID NO: 8 - DNA, 1530 bp, Clostridium ramosum
  modified_base (70)..(70): a, c, t, g, unknown or other
  modified_base (541)..(541): a, c, t, g, unknown or other
  modified_base (1372)..(1372): a, c, t, g, unknown or other
  modified_base (1423)..(1423): a, c, t, g, unknown or other
  modified_base (1471)..(1471): a, c, t, g, unknown or other
  modified_base (1476)..(1482): a, c, t, g, unknown or other
  modified_base (1502)..(1502): a, c, t, g, unknown or other
  modified_base (1508)..(1515): a, c, t, g, unknown or other
  modified_base (1530)..(1530): a, c, t, g, unknown or other
acaatggagagtttgatcctggctcaggatgaacgctggcggcgtgcctaatacatgcaagtcgaacgcnagcacttgtgcttcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctagttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcnagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggtgcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttctcgngccttgtacacaccgcccgtcacaccacgagagttgataacacccgaagcnggtggcctaaccgcaaggaaggagctgtctaaggtgggattgatgatnggggnnnnnnngtaacaaggtatccctacgngaacgnnnnnnnngatcacctcctttcn

SEQ ID NO: 9 - DNA, 1519 bp, Clostridium sardiniense
tttaaattgagagtttgatcctggctcaggacgaacgctggcggcgtgcctaacacatgcaagtcgagcgatgaagtttccttcgggaaacggattagcggcggacgggtgagtaacacgtgggtaacctgcctcatagaggggaatagccttccgaaaggaagattaataccgcataacattgcagtttcgcatgaaacagcaattaaaggagcaatccgctatgagatggacccgcggcgcattagctagttggtaaggtaatggcttaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggtcttcggattgtaaagctctgtctttggggacgataatgacggtacccaaggaggaagccacggctaactacgtgccagcagccgcggtaatacgtaggtggcaagcgttgtccggatttactgggcgtaaagggagcgtaggcggatttttaagtgggatgtgaaatacccgggctcaacctgggtgctgcattccaaactgggaatctagagtgcaggaggggagagtggaattcctagtgtagcggtgaaatgcgtagagattaggaagaacaccagtggcgaaggcgactctctggactgtaactgacgctgaggctcgaaagcgtggggagcaaacaggattagataccctggtagtccacgccgtaaacgatgaatactaggtgtaggggtttcgatacctctgtgccgccgctaacgcattaagtattccgcctggggagtacggtcgcaagattaaaactcaaaggaattgacgggggcccgcacaagtagcggagcatgtggtttaattcgaagcaacgcgaagaaccttacctagacttgacatcttctgcattacccttaatcggggaagtcctttcggggacagaatgacaggtggtgcatggttgtcgtcagctcgtgtcgtgagatgttgggttaagtcccgcaacgagcgcaacccctattgttagttgctaccattaagttgagcactctagcgagactgcccgggttaaccgggaggaaggtggggatgacgtcaaatcatcatgccccttatgtctagggctacacacgtgctacaatggcaagtacagagagatgcaataccgtgaggtggagctaaacttcaaaacttgtctcagttcggattgtaggctgaaactcgcctacatgaagctggagttactagtaatcgcgaatcagcatgtcgcggtgaatacgttcccgggccttgtacacaccgcccgtcacaccatgagagttggcaatacccaaagttcgtgagctaacgcgtaagcgaggcagcgacctaaggtagggtcagcgattggggtgaagtcgtaacaaggtagccgtaggagaacctgcggctggatcacctcctttct

SEQ ID NO: 10 - DNA, 1529 bp, Clostridium scindens
gagagtttgatcctggctcaggatgaacgctggcggcgtgcctaacacatgcaagtcgaacgaagcgcctggccccgacttcttcggaacgaggagccttgcgactgagtggcggacgggtgagtaacgcgtgggcaacctgccttgcactgggggataacagccagaaatggctgctaataccgcataagaccgaagcgccgcatggcgcggcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcattttggatgggcactctggagagactgccagggagaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagttggaatcgctagtaatcgcgaatcagaatgtcgcggtgaatacgttcccgggtcttgtacacaccgcccgtcacaccatgggagtcagtaacgcccgaagccggtgacccaacccgtaagggagggagccgtcgaaggtgggaccgataactggggtgaagtcgtaacaaggtagccgtatcggaaggtgcggctggatcacctccttc

SEQ ID NO: 11 - DNA, 844 bp, Escherichia coli
gcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaac

SEQ ID NO: 12 - DNA, 512 bp, Klebsiella oxytoca
cctgaaagaacctgcctttgtagtgctcacacagattgtctgatgaaaaataagcagtaagaaaatctctgcaggcttgtagctcaggtggttagagcgcacccctgataagggtgaggtcggtggttcaagtccactcaggcctaccaaatttctgctgatgctgcgttgcggcgacactcacatactttaagtatgtttcgtgtcaccacgccttgcctcaacagaaattaaggttgatgagattttaactacgatggggctatagctcagctgggagagcgcctgctttgcacgcaggaggtctgcggttcgatcccgcatagctccaccatcattactgccaaaaacaagaaaacttcagagtgtacctgaaaaggttcactgcgaagttttgctctttaaaaatctggatcaagctgaaaattgaaacgacacacagctaatgtgtgttcgagtctctcaaattttcgcgacacgatgatgtttcacgaaacatcttcgggttgtga

SEQ ID NO: 13 - DNA, 1497 bp, Parabacteroides distasonis
caatttaaacaacgaagagtttgatcctggctcaggatgaacgctagcgacaggcttaacacatgcaagtcgaggggcagcggggtgtagcaatacaccgccggcgaccggcgcacgggtgagtaacgcgtatgcaacttgcctatcagagggggataacccggcgaaagtcggactaataccgcatgaagcagggatcccgcatgggaatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggttaggctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactggtgactggggctaagtcgtaacaag

SEQ ID NO: 14 - DNA, 1506 bp, Prevotella melaninogenica
  modified_base (36)..(36): a, c, t, g, unknown or other
gatgaacgctagctacaggcttaacacatgcaagtngaggggaaacggcattgagtgcttgcactctttggacgtcgaccggcgcacgggtgagtaacgcgtatccaaccttcccattactgtgggataacctgccgaaaggcagactaataccgcatagtcttcgatgacggcatcagatttgaagtaaagatttatcggtaatggatggggatgcgtctgattagcttgttggcggggtaacggcccaccaaggcaacgatcagtaggggttctgagaggaaggtcccccacattggaactgagacacggtccaaactcctacgggaggcagcagtgaggaatattggtcaatggacggaagtctgaaccagccaagtagcgtgcaggatgacggccctatgggttgtaaactgcttttgtatggggataaagttagggacgtgtccctatttgcaggtaccatacgaataaggaccggctaattccgtgccagcagccgcggtaatacggaaggtccaggcgttatccggatttattgggtttaaagggagcgtaggctggagattaagtgtgttgtgaaatgtagacgctcaacgtctgaattgcagcgcatactggtttccttgagtacgcacaacgttggcggaattcgtcgtgtagcggtgaaatgcttagatatgacgaagaactccgattgcgaaggcagctgacgggagcgcaactgacgcttaagctcgaaggtgcgggtatcaaacaggattagataccctggtagtccgcacagtaaacgatggatgcccgctgttggtacctggtatcagcggctaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttagagataatgacgcccttcggggtctctgtgaaggtgctgcatggttgtcgtcagctcgtgccgtgaggtgtcggcttaagtgccataacgagcgcaacccctctcttcagttgccatcaggttaagctgggcactctggagacactgccaccgtaaggtgtgaggaaggtggggatgacgtcaaatcagcacggcccttacgtccggggctacacacgtgttacaatggccggtacagagggacggtgtaatgcaaattgcatccaatcttgaaagccggtcccagttcggactggggtctgcaacccgaccccacgaagctggattcgctagtaatcgcgcatcagccatggcgcggtgaatacgttcccgggccttgtacacaccgcccgtcaagccatgaaagccgggggtgcctgaagtccgtgaccgcaaggatcggcctagggcaaaactggtgattggggctaagtcgtaacaaggtagccgtaccggaaggtgcggctggaacacctcctttct

SEQ ID NO: 15 - DNA, 1497 bp, Proteus mirabilis
tgatcctggctcagattgaacgctggcggcaggcctaacacatgcaagtcgagcggtaacaggagaaagcttgctttcttgctgacgagcggcggacgggtgagtaatgtatggggatctgcccgatagagggggataactactggaaacggtggctaataccgcataatgtctacggaccaaagcaggggctcttcggaccttgcactatcggatgaacccatatgggattagctagtaggtggggtaaaggctcacctaggcgacgatctctagctggtctgagaggatgatcagccacactgggactgagacacggcccagactcctacgggaggcagcagtggggaatattgcacaatgggcgcaagcctgatgcagccatgccgcgtgtatgaagaaggccttagggttgtaaagtactttcagcggggaggaaggtgataaggttaatacccttatcaattgacgttacccgcagaagaagcaccggctaactccgtgccagcagccgcggtaatacggagggtgcaagcgttaatcggaattactgggcgtaaagcgcacgcaggcggtcaattaagtcagatgtgaaagccccgagcttaacttgggaattgcatctgaaactggttggctagagtcttgtagaggggggtagaattccatgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttagaggttgtggtcttgaaccgtggcttctggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagcgaatcctttagagatagaggagtgccttcgggaacgctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtagc

(19) United States
(12) Patent Application Publication
(10) Pub. No.: US 2022/0096566 A1
(43) Pub. Date: Mar. 31, 2022

(54) THERAPEUTIC MICROBIOTA FOR THE TREATMENT AND/OR PREVENTION OF DYSBIOSIS

(71) Applicants: THE BRIGHAM AND WOMEN'S HOSPITAL, INC., Boston, MA (US); THE CHILDREN'S MEDICAL CENTER CORPORATION, Boston, MA (US)

(72) Inventors: Lynn BRY, Jamaica Plain, MA (US); Talal A. CHATILA, Belmont, MA (US); Georg GERBER, Newton, MA (US); Azza ABDEL-GADIR, Cambridge, MA (US); Rima RACHID, Belmont, MA (US)

(73) Assignee: THE BRIGHAM AND WOMEN'S HOSPITAL, INC., Boston, MA (US)

(21) Appl. No.: 17/292,482

(22) PCT Filed: Nov. 8, 2019
(86) PCT No.: PCT/US2019/060504

§ 371 Date: May 10, 2021

Related U.S. Application Data
(60) Provisional application No. 62/758,276, filed on Nov. 9, 2018.

(51) Int. Cl. A61K 35/741; A61K 45/06; A61P 1/14

(52) U.S. Cl. CPC A61K 35/741; A61P 1/14; A61K 45/06

(57) ABSTRACT

Disclosed are methods and compositions for the prevention and treatment of dysbiosis and associated conditions. In particular, described herein are compositions of microbial consortia, including minimal microbial consortia, that can prevent and/or cure dysbiosis and associated conditions. In certain embodiments, the microbial consortia comprise certain members of the taxa Clostridiales, Bacteroidetes, Prevotella, and/or Parabacteroides.

### CROSS-REFERENCE TO RELATED APPLICATION

This application claims benefit under 35 U.S.C. § 119(e) of U.S. Provisional Application No. 62/758,276 filed Nov. 9, 2018, the content of which is incorporated herein by reference in its entirety.

### GOVERNMENT SUPPORT

This invention was made with Government Support under Grant Nos. P30-DK-034854, 1R56AI117983 and 1R01AI126915 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Oct. 31, 2019, is named 043214-093940WOPT_SL.txt and is 33,281 bytes in size.

### FIELD OF THE DISCLOSURE

The present disclosure relates to the treatment and/or prevention of dysbiosis and associated conditions.

### BACKGROUND

Growing evidence indicates that the microbial flora is a key environmental influence on a myriad of physiological signaling mechanisms in the body. Furthermore, the microbial flora can influence the development of disease.

### SUMMARY

Provided herein are methods and compositions for the treatment and/or prevention of dysbiosis and associated diseases or disorders, including but not limited to inflammatory diseases or disorders, metabolic diseases or disorders, and atopic diseases or disorders. The methods and compositions described herein are based, in part, on the discovery that altered intestinal microbiota (e.g., from antibiotic treatments, C-section births, diet etc.) can promote dysbiosis and inflammatory and atopic diseases while some combinations of microbes can prevent and/or ameliorate or cure dysbioses, and inflammatory and/or atopic diseases.

In one aspect, described herein is a pharmaceutical composition comprising a microbial consortium of culturable species, and a pharmaceutically acceptable carrier, wherein the consortium is comprised in at least two of the preparations selected from the group consisting of: (i) a preparation of a viable, culturable, anaerobic gut bacterial strain(s) that expresses exopolysaccharide, lipoteichoic acid (LTA), lipopolysaccharide (LPS) or other microbial adjuvant molecules that promote the development of regulatory T cells (T_{reg}); (ii) a preparation of a viable, culturable, anaerobic gut bacterial strain(s) that produces butyrate and/or propionate fermentation products via fermentation of carbohydrates and other carbon sources in the gut lumen; (iii) a preparation of one or more viable, culturable, anaerobic gut bacterial strains that alone or in combination performs the full complement of bile acid transformations; (iv) a preparation of a viable, culturable, anaerobic gut bacterial strain that produces compounds capable of stimulating the aryl hydrocarbon receptor (AhR) receptor pathway in gut epithelial cells, antigen presenting cells and/or T cells to stimulate development of regulatory T cell responses; (v) a preparation of a viable, culturable, anaerobic gut bacterial strain(s) that produces compounds capable of stimulating the pregnane X receptor with beneficial effects upon gut barrier function and/or development of regulatory T cell responses; (vi) a preparation of a viable, culturable, anaerobic gut bacterial strain(s) that produces compounds capable of stimulating the RORgamma (RAR-related orphan receptor gamma) pathways to stimulate development of regulatory T cell responses via direct stimulation or RORgamma-activated pathways in gut antigen presenting cells and/or epithelial cells that then stimulate regulatory T cell responses; (vii) a preparation of viable, culturable, anaerobic gut bacterial strain(s) that stimulates host production of mucins and complex glycoconjugates that improve gut barrier function and colonization by protective commensal species; (viii) a preparation of a viable, culturable, anaerobic gut bacterial strain(s) that alters the gut luminal environment to reduce the deleterious activities of dysbiotic species promoting development of unhealthy allergic T cell responses to, for example, food antigens; (ix) a preparation of a viable, culturable, anaerobic gut bacterial strain(s) that alters the gut luminal environment to promote improved colonization by other members of the administered consortium for any of the above stated effects, and/or colonization by existing beneficial species in the patients underlying microbiota; (x) a preparation of a viable, culturable, anaerobic gut bacterial strain(s) that promotes the colonization or growth of a bacterial strain in a preparation of (i)-(ix) above, in vivo.

In another aspect, described herein is a pharmaceutical composition comprising:
a. a preparation comprising a microbial consortium of isolated bacteria that comprises two to twenty species of viable gut bacteria, at least two of which are selected from the group consisting of: Bacteroides vulgatus, Parabacteroides distasonis, and Prevotella melaninogenica, in an amount sufficient to treat or prevent a dysbiosis to an individual in need thereof, and
b. a pharmaceutically acceptable carrier.

In another aspect, described herein is a pharmaceutical composition comprising:
a. a preparation comprising at least two species of isolated, viable, anaerobic gut bacteria selected from the group consisting of: Bacteroides vulgatus, Parabacteroides distasonis, and Prevotella melaninogenica, in an amount sufficient to treat or prevent dysbiosis when administered to an individual in need thereof, and
b. a pharmaceutically acceptable carrier.

In another aspect, described herein is a pharmaceutical composition comprising:
a. a preparation comprising at least three species of isolated, viable, anaerobic gut bacteria comprising: Bacteroides vulgatus, Parabacteroides distasonis, and Prevotella melaninogenica, in an amount sufficient to treat or prevent dysbiosis when administered to an individual in need thereof, and
b. a pharmaceutically acceptable carrier.

In another aspect, described herein is a pharmaceutical composition comprising:
a. a preparation comprising at least four species of isolated, viable, anaerobic gut bacteria selected from the group consisting of: Bacteroides fragilis, Bacteroides ovatus, Bacteroides vulgatus, Parabacteroides distasonis, and Prevotella melaninogenica, in an amount sufficient to treat or prevent a dysbiosis when administered to an individual in need thereof, and
b. a pharmaceutically acceptable carrier.

In another aspect, described herein is a pharmaceutical composition comprising:
a. a preparation comprising isolated, viable, anaerobic gut bacteria including each of Bacteroides fragilis, Bacteroides ovatus, Bacteroides vulgatus, Parabacteroides distasonis, and Prevotella melaninogenica, in an amount sufficient to treat or prevent dysbiosis when administered to an individual in need thereof, and
b. a pharmaceutically acceptable carrier.

In another aspect, described herein is a method for treating, or preventing a dysbiosis in a subject, the method comprising: administering to a subject a pharmaceutical composition described herein, thereby treating, or preventing dysbiosis in the subject.

In another aspect, described herein is a method for the treatment, or prevention of gut inflammation or a metabolic disease or disorder, the method comprising: administering to a subject a pharmaceutical composition described herein, thereby treating, or preventing the gut inflammation or metabolic disease or disorder in the subject.

In another aspect, described herein is a method for the treatment, or prevention of an atopic disease or disorder, the method comprising: administering to a subject a pharmaceutical composition described herein, thereby treating, or preventing the atopic disease or disorder in the subject.

In another aspect, described herein is a method for preventing the onset of an allergy in a subject, the method comprising: administering to a subject a composition of any one of the pharmaceutical compositions described herein, thereby preventing the onset of an allergy in the subject.

In another aspect, described herein is a method for reducing or eliminating a subject's immune reaction to an allergen, the method comprises: administering to a subject a pharmaceutical composition comprising a microbial consortium as described herein, thereby reducing or eliminating a subject's immune reaction to an allergen.

In another aspect, described herein is a method of monitoring a subject's microbiome, the method comprising: determining the presence and/or biomass in a biological sample obtained from a subject, and wherein if at least two or more species selected from the group consisting of Bacteroides fragilis, Bacteroides ovatus, Bacteroides vulgatus, Parabacteroides distasonis, and Prevotela melaninogenica, are absent or low relative to a reference, the subject is treated with a pharmaceutical composition comprising a microbial consortium as described herein.

In another aspect, described herein is a method of treating atopic disease or disorder in an individual in need thereof, the method comprises administering a pharmaceutical composition comprising a microbial consortium as described herein to the individual.

In another aspect, described herein is a method of reducing the number or activity of Tₕ2 cells in a tissue of an individual in need thereof, the method comprising administering a pharmaceutical composition comprising a microbial consortium as described herein to the individual.

In another aspect, described herein is a synergistic microbial composition comprising:
a. a first microbial consortium consisting essentially of two to five species of viable gut bacteria selected from the group consisting of: Bacteroides fragilis, Bacteroides ovatus, Bacteroides vulgatus, Parabacteroides distasonis, and Prevotella melaninogenica; and
b. a second microbial consortium consisting essentially of one to six species of viable gut bacteria selected from the group consisting of: Clostridium ramosum, Clostridium scindens, Clostridium hiranonsis, Clostridium bifermentans, Clostridium leptum, and Clostridium sardiniensis, wherein one or more members of the second microbial consortium increases the colonization and/or persistence of one or more members of the first microbial consortium in a mammalian host.

In another aspect, described herein is a synergistic microbial composition comprising:
a. a first microbial consortium consisting essentially of one to six species of viable gut bacteria selected from the group consisting of: Clostridium ramosum, Clostridium scindens, Clostridium hiranonsis, Clostridium bifermentans, Clostridium leptum, and Clostridium sardiniensis; and
b. a second microbial consortium consisting essentially of two to five species of viable gut bacteria, wherein the species of viable gut bacteria are selected from the group consisting of: Bacteroides fragilis, Bacteroides ovatus, Bacteroides vulgatus, Parabacteroides distasonis, and Prevotella melaninogenica, wherein one or more members of the second microbial consortium increases the colonization and/or persistence of one or more members of the first microbial consortium in a mammalian host.

In one embodiment of any of the aspects, including methods and compositions, the pharmaceutical composition or the microbial consortium further comprises at least one of Bacteroides fragilis and Bacteroides ovatus. In further embodiments, the pharmaceutical composition or the microbial consortium further comprises Bacteroides fragilis and Bacteroides ovatus. In further embodiments, the pharmaceutical composition or the microbial consortium further comprises each of Bacteroides fragilis, Bacteroides ovatus, Bacteroides vulgatus, Parabacteroides distasonis, and Prevotella melaninogenica. In further embodiments, the pharmaceutical composition or the microbial consortium further comprises one or more of the bacteria in TABLE 4.

In another embodiment of any of the aspects, including methods and compositions, the pharmaceutical composition or the microbial consortium comprises at least three or at least four species selected from the group consisting of: Bacteroides fragilis, Bacteroides ovatus, Bacteroides vulgatus, Parabacteroides distasonis, and Prevotella melaninogenica. In further embodiments, the microbial consortium comprises each of the species Bacteroides fragilis, Bacteroides ovatus, Bacteroides vulgatus, Parabacteroides distasonis, and Prevotella melaninogenica.

In another embodiment of any of the aspects, including methods and compositions, the pharmaceutical composition or the microbial consortium further comprises at least one, at least two, at least three, at least four, or at least five of species selected from the group consisting of: Clostridium ramosum, Clostridium scindens, Clostridium hiranonsis, Clostridium bifermentans, Clostridium leptum, and Clostridium sardiniensis. In further embodiments, the pharmaceutical composition or the microbial consortium further comprises each of the species Clostridium ramosum, Clostridium scindens, Clostridium hiranonsis, Clostridium bifermentans, Clostridium leptum, and Clostridium sardiniensis.

In another embodiment of any of the aspects, including methods and compositions, the pharmaceutical composition or the microbial consortium comprises: Bacteroides fragilis, Bacteroides ovatus, Bacteroides vulgatus, Parabacteroides distasonis, Prevotella melaninogenica, Clostridium ramosum, Clostridium scindens, Clostridium rhiranonsis, Clostridium bifermentans, Clostridium leptum, and Clostridium sardiniensis.

In another embodiment of any of the aspects, including methods and compositions, the pharmaceutical composition or the microbial consortium consists essentially of: Bacteroides vulgatus, Parabacteroides distasonis, and Prevotella melaninogenica. In further embodiments, the pharmaceutical composition or the microbial consortium consists essentially of Bacteroides fragilis, Bacteroides ovatus, Bacteroides vulgatus, Parabacteroides distasonis, and Prevotella melaninogenica. In further embodiments, the pharmaceutical composition or the microbial consortium consists essentially of: Clostridium ramosum, Clostridium scindens, Clostridium hiranonsis, Clostridium bifermentans, Clostridium leptum, Clostridium sardiniensrs, Bacteroides fragilis, Bacteroides ovatus, Bacteroides vulgatus, Parabacteroides distasonis, and Prevotella melaninogenica. In further embodiments, the pharmaceutical composition or the microbial consortium consists essentially of: Clostridium ramosum, Clostridium scindens, Clostridium hiranonsis, Clostridium bifermentans, Clostridium leptum, Clostridium sardiniensis, Bacteroides vulgatus, Parabacteroides distasonis, and Prevotella melaninogenica.

In another embodiment of any of the aspects, including methods and compositions, the pharmaceutical composition or microbial consortium comprises at least two, at least three, at least four, or at least five bacterial species, each comprising a 16S rDNA sequence at least 97% identical to a 16S rDNA sequence present in a reference strain operational taxonomic unit, the reference strains selected from the species: Bacteroides fragilis, Bacteroides ovatus, Bacteroides vulgatus, Parabacteroides distasonis and Prevotella melaninogenica.

In another embodiment of any of the aspects, including methods and compositions, the pharmaceutical composition or microbial consortium does not comprise any of the Species Escherichia coli, Klebsiella pneumoniae, Proteus mirabilis, Enterobacter cloacae, Bilophila wadsworthia, Alistipes onderdonkii, Desulfovibrio species, Lactobacillus johnsonii, or Parasutterella excrementihominis. In further embodiments, pharmaceutical composition or microbial consortium does not comprise bacteria of the Genera Bilophila, Enterobacter, Escherichia, Klebsiella, Proteus, Alistipes, Blautia, Desulfovibrio, and Parasutterella. In further embodiments, pharmaceutical composition or microbial consortium does not comprise bacteria of the Families Desulfovibrionaceae, Enterobacteriaceae, Rikenellaceae, and Sutterellaceae, Lactobacillaceae, or Enterbacteriaceae. In further embodiments, the pharmaceutical composition or microbial consortium does not comprise bacteria of the Order Burkholdales, Desulfovibrionales, or Enterobacteriales.

In another embodiment of any of the aspects, including methods and compositions, the pharmaceutical composition or microbial consortium comprises at least three or at least four species of viable non-pathogenic gut bacteria. In further embodiments, the microbial consortium comprises at least two and up to eleven species of viable non-pathogenic gut bacteria.

In another embodiment of any of the aspects, including methods and compositions, the viable gut bacteria are anaerobic gut bacteria. In another embodiment, the viable gut bacteria are human gut bacteria. In further embodiments, the species of viable gut bacteria are isolated and/or purified from a subject known to be tolerant to a selected allergen (e.g., a food allergen). In further embodiments, the species of viable gut bacteria are prepared by culture under anaerobic conditions. In further embodiments, the species of viable gut bacteria are formulated to maintain anaerobic conditions. In further embodiments, the anaerobic conditions are maintained by one or more of the following: (i) oxygen impermeable capsules, (ii) addition of a reducing agent including N-acetylcysteine, cysteine, or methylene blue to the composition, or (iii) use of spores for organisms that sporulate.

In another embodiment of any of the aspects, including methods and compositions, the species of viable gut bacteria are present in substantially equal biomass. In further embodiments, the biomass of each of the microbes in the administered compositions is greater than the biomass of each of the microbes relative to a reference. In further embodiments, the pharmaceutical composition is formulated to deliver a dose of at least 1×10⁷ colony-forming units (CFUs). In further embodiments, the pharmaceutical composition is formulated to deliver a dose of at least 1×10⁸ CFUs. In further embodiments, the pharmaceutical composition is formulated to deliver a dose of at least 1×10⁹ CFUs. In further embodiments, the pharmaceutical composition is formulated to deliver a dose of at least 1×10¹⁰ CFUs. In further embodiments, the pharmaceutical composition is formulated to deliver a dose of at least 1×10¹¹ CFUs. In further embodiments, the pharmaceutical composition is formulated to deliver a dose of at least 1×10¹² CFUs. In further embodiments, the pharmaceutical composition is formulated to deliver at least 1×10⁹ CFUs in less than 30 capsules per one-time dose.

In another embodiment of any of the aspects, including methods and compositions, the pharmaceutical composition is formulated to deliver the viable bacteria to the small intestine. In further embodiments, the pharmaceutical composition is enteric coated. In further embodiments, the enteric coating comprises a polymer, nanoparticle, fatty acid, shellac, or a plant fiber. In further embodiments, the pharmaceutically acceptable carrier comprises an enteric coating composition that encapsulates the microbial consortium. In further embodiments, the pharmaceutically acceptable carrier comprises a capsule, gel, pastille, tablet or pill. In further embodiments, the pharmaceutical composition further comprises a prebiotic composition. In further embodiments, the pharmaceutical composition and/or microbial consortium are frozen for storage. In further embodiments, the microbial consortium is encapsulated, lyophilized, formulated in a food item, or is formulated as a liquid, gel, fluid-gel, or nanoparticles in a liquid.

In another embodiment of any of the aspects, including methods and compositions, the dysbiosis described herein is associated with an inflammatory disease or a metabolic disease or disorder. In further embodiments of any of the aspects, the dysbiosis is associated with an atopic disease or disorder.

In another embodiment of any of the aspects, including methods and compositions, the inflammatory disease is selected from the group consisting of: a gastrointestinal infection, a respiratory infection, a kidney infection, pancreatitis, gingivitis, periodontitis, inflammatory bowel disease (IBD), Crohn's disease (CD), ulcerative colitis (UC), gastritis, enteritis, esophagitis, gastroesophageal reflux disease (GERD), celiac disease, diverticulitis, food intolerance, ulcer, infectious colitis, irritable bowel syndrome, and cancer. In further embodiments, the atopic disease or disorder is selected from the group consisting of: food allergy, asthma, eczema, and rhinoconjunctivitis. In further embodiments, the allergy described herein is a food allergy. In further embodiments, the food allergy comprises allergy to say, wheat, eggs, dairy, peanuts, tree nuts, shellfish, fish, mushrooms, stone fruits and/or other fruits.

In another embodiment of any of the aspects, including methods and compositions, the subject is pretreated with an antibiotic. In further embodiments, the subject is pretreated with a fasting period not longer than 24 hours. In further embodiments, the subject is pretreated with a probiotic. In further embodiments, the subject is a human subject. In further embodiments of any of the aspects, the subject is an infant (e.g., the subject is under the age of 2 years old); a child (e.g., the subject is age 2 to under 5 years old); an adolescent (e.g., age 5 to under 12 years old); a teenager (e.g., age 12 to under 18 years old); an adult (e.g., age 18 to under 65); or an elderly adult (e.g., the subject is over the age of 65 years old). In further embodiments, the subject is under the age of 2 years old. In further embodiments, the subject is age 2 to under 5 years old. In further embodiments, the subject is age 5 to under 8 years old. In further embodiments, the subject is age 5 to under 12 years old. In further embodiments, the subject is age 12 to under 18 years old. In further embodiments, the subject is age 18 to under 65 years old. In further embodiments, the subject is over age 65 years old.

In another embodiment of any of the aspects, including methods and compositions, the the pharmaceutical composition or the administering further comprises a prebiotic composition. In further embodiments, the pharmaceutical composition is administered by oral administration, enema, suppository, or orogastric tube. In further embodiments, the pharmaceutical composition is administered before the first exposure to a potential food allergen. In further embodiments, the pharmaceutical composition is administered upon clinical signs of atopic symptoms. In further embodiments, the pharmaceutical composition is administered to an individual diagnosed with an allergy. In further embodiments, the allergy is a food allergy.

In another embodiment of any of the aspects, including methods and compositions, the the pharmaceutical composition or the treatment administered prevents and/or reverses Tₕ2 programming of T_{regs} and other mucosal T cell populations. In further embodiments, the administration shifts the balance of Tₕ1/Tₕ2 cells towards Tₕ1 T cells. In further embodiments, the pharmaceutical composition or the administration reduces the number or activity of Tₕ2 T cells.

In another embodiment of any of the aspects, including methods and compositions, the individual in need or the methods described herein further comprise a step of diagnosing the subject or individual as having or as likely to develop an inflammatory disease or an atopic disease or disorder. In further embodiments, the individual or the methods described herein further comprise a step of diagnosing the subject or individual as having or as likely to develop an allergy (e.g., a food allergy).

In another embodiment of any of the aspects, the methods described herein further comprise a step of testing a biological sample (e.g., a fecal sample) from the subject for the presence and/or levels of one or more of the bacteria in the microbial consortium. In further embodiments, the methods described herein further comprise predicting that a subject will have an immune response to an allergen when the at least two members are absent, the biomass of the at least two members is low relative to a reference, or at least one member of a dysbiotic species is present or elevated relative to a reference. In further embodiments, the methods described herein are repeated at least one additional time. In further embodiments, the biological sample described herein is a fecal sample. In further embodiments, the biological sample is a tissue. In further embodiments, the tissue is a gut tissue.

In another embodiment of any of the aspects, the methods described herein further comprise a step of diagnosing the subject or individual as having an IgE-mediated allergy. In further embodiments, the Ig-E-mediated allergy is a food allergy. In further embodiments, the food allergy comprises allergy to soy, wheat, eggs, dairy, peanuts, tree nuts, shellfish, fish, mushrooms, stone fruits or other fruits.

### BRIEF DESCRIPTION OF THE FIGURES

FIG. 1 shows a schematic representation of tolerance failure in atopic disease, such as food allergy, which is due to a failure of oral tolerance to food antigens. The pathophysiological mechanism of food allergy is associated with Tₕ2 immunity and allergen-specific IgE responses. T regulatory cells (T_{regs}) generally suppress type-2 innate lymphoid cells (ILC2), Tₕ2 cell activation, mast cell activation, and dendritic cell (DC) activation.

FIG. 2 shows a schematic representation of an atopic disease experimental model: Il4raF709 mutant mice, which are prone to development of food allergy. A mutation in the Type I IL-4 receptor, ITIM, (Y709 to F709) results in a gain of function of the IL-4 receptor.

FIG. 3 shows a schematic representation of an exemplary ovalbumin sensitization protocol. IL4raF709 mutant mice and WT mice were challenged with chicken egg ovalbumin (OVA) along with the mucosal adjuvant staphylococcal entero-toxin B (SEB), followed by a subsequent challenge with OVA. Mutant and WT mice were monitored for changes in core body temperature, total serum IgE, Ova-specific serum IgE, mucosal mast cell protease-1 (MMCP-1) levels, and mast cell counts.

FIG. 4A-4C shows ovalbumin (OVA)-induced food allergic reaction in Il4raF709 mice. FIG. 4A shows the core body temperature change of WT and Il4raF709 mice in response to saline or OVA-SEB over time (minutes). Il4raF709 mice treated with OVA-SEB exhibited a statistically significant drop in core body temperature, indicative of anaphylaxis. WT mice treated with OVA-SEB and WT mice and Il4raF709 mice treated with saline (PBS) did not have significant changes in core body temperature. FIG. 4B shows total serum IgE (i), number of (Nbr) mast cells/LPF levels (ii), OVA-specific IgE levels (iii), and MMCP-1 levels (iv) in WT and Il4raF709 mice treated with saline or OVA-SEB. FIG. 4C shows flow cytometry of immune cells isolated from WT and Il4raF709 mice. Il4raF709 mice treated with OVA-SEB exhibited increases in the percentage (%) of CD4+ IL-4+ T cells and the number (Nbr) of CD4+ IL-4+ T cells compared to WT mice treated with OVA-SEB.

FIG. 5A-5C shows allergen-specific T_{reg} cell deficiency in allergic Il4raF709 mice. FIG. 5A demonstrates representative flow cytometry plots of CD4+Foxp3+ T_{reg} cells. FIG. 5B demonstrates that the number of CD4+Fox3p+ T_{reg} cells in the small intestine (SI), mesenteric lymph nodes (MLN), and spleen are significantly decreased in Il4raF709 mice treated with OVA-SEB compared with WT mice treated with OVA-SEB. In the small intestine, Il4raF709 mice exhibited reductions in the number of CD4+Fox3p+ T_{reg} cells compared to WT mice treated with saline. FIG. 5C shows analysis of CD4+Foxp3+ T_{reg} cell proliferation following OVA-SEB or saline treatment in WT and Il4raF709 mice. CD4+Foxp3+ T_{reg} cell proliferation was reduced in Il4raF709 mice treated with OVA-SEB compared with WT mice treated with OVA-SEB.

FIG. 6 demonstrates that the oral allergic sensitization in F709 mutant mice is associated with dysbiosis. Il4raF709 mice exhibited reductions in several phyla of bacteria in the small intestine.

FIG. 7 provides a schematic representation of an exemplary protocol to test whether the microbiota of sensitized Il4raF709 mice transmit susceptibility to food allergy. Microbiota from WT or IL4raF704 mice are transferred to WT germ free (GF) mice followed by OVA challenge at 8-weeks post-transfer.

FIG. 8 shows that the microbiota of Il4raF709 mice promote allergic sensitization and anaphylaxis in germ free (GF) mice. The left graph shows that the core body temperature of WT germ free mice following OVA challenge and administered Il4raF709 flora exhibited a drop in core body temperature compared with WT mice that receive WT microbial flora. This result is similar to that observed in the Il4raF709 mice challenged with OVA. The right graph demonstrates that GF WT mice that were administered Il4raF709 flora exhibit a significant increase in mMCP-1 levels after re-challenge with OVA compared with GF WT mice that received WT flora.

FIG. 9 shows the microbiota of Il4raF709 mice promote allergic sensitization and anaphylaxis. The left graphs show analysis of CD3+ CD4+ T cells in WT GF mice that received WT or Il4raF709 flora. The bar graphs (right) show that GF WT mice that were administered Il4raF709 flora exhibited a significant increase in the percentage of IL4+ cells compared with WT GF mice administered WT microbial flora.

FIG. 10 shows a schematic representation of an exemplary protocol to determine whether microbiota of food tolerant mice transmit protection against food allergy.

FIGS. 11A-11D demonstrate that the microbiota of food tolerant mice protect against allergic sensitization and anaphylaxis in a genetically susceptible host. FIG. 11A shows the change in core body temperature over time following OVA challenge in Il4raF709 mice that were administered WT or Il4raF709 microbial flora. Il4raF709 mice that were administered WT microbial flora exhibited protection from anaphylaxis. FIG. 11B shows total IgE levels (left) and OVA-specific IgE levels (right) for Il4raF709 mice that were administered WT or Il4raF709 microbial flora. FIG. 11C shows flow cytometry analysis for IL-4+ and IFNγ T cells isolated after OVA challenge from Il4raF709 mice that were administered WT or Il4raF709 microbial flora. FIG. 11D shows the percentage of OVA-specific CD4+L-4+ T cells and CD4+IFNγ+ T cells isolated from Il4raF709 mice that were administered WT or Il4raF709 microbial flora. Il4raF709 mice that were administered WT microbial flora exhibited significant decreases in total IgE, OVA-IgE, and CD4+IL-4+ T cells compared with Il4raF709 mice that were administered Il4raF709 microbial flora.

FIGS. 12A-12D shows the microbiota of food tolerant mice promotes the formation of allergen-specific T_{reg} cells. FIG. 12A shows flow cytometry analysis of CD4+Fox3p+ T_{reg} cells isolated from Il4raF709 mice that were administered WT or Il4raF709 microbial flora. FIG. 12B shows that the Il4raF709 mice that were administered WT microbial flora exhibited significant increases in the percentage and number (Nbr) of CD4+Fox3p+ T_{reg} cells when compared with Il4raF709 mice that were administered Il4raF709 microbial flora. FIGS. 12C-12D shows flow cytometry analysis of Fox3p+ T_{regs} that were actively proliferating using violet proliferative dye. FIG. 12D confirms that Il4raF709 mice that were administered WT microbial flora exhibited an increase in the percentage of proliferating CD4+Fox3p+ T_{regs} compared with Il4raF709 mice that were administered Il4raF709 microbial flora.

FIG. 13 shows a graphical visualization of relative abundances of phyla following ovalbumin (OVA) treatment at 8 weeks for WT (top) and Il4raF709 mice (bottom).

FIG. 14 shows a table of selected OTUs demonstrating differences between WT and F709 mice challenged with OVA. The table shows OTUs in the duodenum, jejunum, and ileum

FIG. 15 shows an exemplary protocol for determining whether treatment with defined bacterial mixes will protect against food allergy. Il4raF709 mutant mice were given antibiotics for 7 days prior to the start of the protocol.

FIGS. 16A-16D shows that Clostridia and Bacteroidetes protect against development of allergen-specific responses and anaphylaxis. FIG. 16A shows change in core body temperature following OVA challenge of Il4raF709 mice treated with Clostridia, Proteobacteria, or Bacteroidetes compared with Il4raF709 mice that did not receive bacterial treatment. Mutant mice treated with Bacteroidetes and Clostridia were protected from a drop in core body temperature following OVA challenge. FIG. 16B shows the number of mast cells, and MMCP-1 levels in Il4raF709 mice administered Clostridia, Proteobacteria, or Bacteroidetes compared with no bacterial treatment. Mutant mice treated with Bacteroidetes and Clostridia exhibited reductions in the number of mast cells and MMCP-1 levels after OVA-challenge compared with mutant mice that were not treated with bacteria or those administered Proteobacteria. FIG. 16C shows jejunal mast cells in OVA-challenged Il4raF709 mice treated with Clostridia, Proteobacteria, or Bacteroidetes compared with no bacterial treatment. FIG. 16D shows total IgE and OVA-specific IgE levels in OVA-challenged Il4raF709 mice treated with Clostridia, Proteobacteria, or Bacteroidetes compared with no bacterial treatment.

FIG. 17A-17D shows oral allergic sensitization is associated with T_{reg} cell Tₕ2 reprogramming. FIG. 17A shows flow cytometry analysis of MLN CD3+ CD4+ T cells from OVA sensitized WT and Il4raF709 mice for IL-4 and Foxp3 markers. FIG. 17B shows the percentage (top) of IL-4+ CD4+Foxp3+ T cells in WT and Il4raF709 mice sensitized with OVA-SEB or treated with PBS. FIG. 17C shows flow cytometry analysis of GATA3+ T cells in WT and Il4raF709 mice sensitized with OVA-SEB or controls treated with PBS. Number and percentage of GATA3+ CD4+Foxp3+ cells are shown at right. FIG. 17D shows flow cytometry analysis of IRF-4+ T cells in WT and Il4raF709 mice sensitized with OVA-SEB or controls treated with PBS. Number and percentage of IRF-4+ CD4+Foxp3+ cells are shown at right.

FIG. 18A-18F shows deletion of Il4/Il13 in T_{reg} cells protects against food allergy. FIG. 18A shows fold change in IL-4 in CD4+FoxP3− versus CD4+FoxP3+ cells in Il4raF709 mice versus Il4raF709 IL4^{-/-}, IL-13^{-/-} mice. FIG. 18B change in body core temperature after OVA challenge over time for Il4raF709 mice Il4raF709 IL4^{-/-}, IL-13^{-/-} mice. FIG. 18C shows total IgE, OVA-specific IgE, MMCP-1 level, and mast cell number in Il4raF709 mice and Il4raF709 IL4^{-/-}, IL-13^{-/-} mice. FIG. 18D-FIG. 18E show number (bottom), and percentage (top) of CD4+ Fox3pd+ (18D), percentage of IRF-4 (18E, bottom) and GATA3+(18E, top) CD4+ Foxp3+ cells in Il4raF709 versus Il4raF709 IL4^{-/-}, IL-13^{-/-} mice. FIG. 18F shows number (bottom) and percentage (top) of IL-4+ CD4+ Foxp3− cells in the Il4raF709 mice versus Il4raF709 IL4^{-/-}, IL-13^{-/-} mice.

FIG. 19 shows reduced Tₕ2-skewed T_{reg} phenotype indicates that Clostridia and Bacteroidetes have different molecular mechanisms of action. Left: number and percentage of CD3+CD4+Foxp3+ T_{reg} cells in OVA-challenged Il4raF709 mice treated with no bacteria and mice treated with Clostridia, Proteobacteria, or Bacteroidetes. Center: FACs plots and graphical representation of CD3+CD4+Foxp3+GATA3+ cells in Il4raF709 mice treated with no bacteria and mice treated with Clostridia, Proteobacteria, or Bacteroidetes. Clostridia and Bacteroidetes each protect, but show significant differences in relative IL-4 and GATA3 expression in T_{reg}.

FIG. 20A-20D demonstrates that short chain fatty acid (SCFA) therapy does not rescue food allergy in Il4raF709 mice. FIG. 20A shows SCFAs, isovalerate, valerate, acetate, propionate, and butryate (mM) concentrations measured in WT and Il4raF709 mice administered saline or OVA-SEB. FIG. 20B shows change in core body temperature in WT and Il4raF709 mice treated with PBS or OVA-SEB with and without SCFA treatment. FIG. 20C shows total IgE and OVA-specific IgE in WT and Il4raF709 mice treated with OVA-SEB, PBS-SCFA, and OVA-SEB+SCFAs. FIG. 20D shows flow cytometry analysis for CD4+Foxp3+ cells isolated from the small intestine of WT and Il4raF709 mice treated with OVA-EB, PBS-SCFAs, and OVA-SEB-SCFAs.

FIGS. 21A-21I demonstrates that a defined consortium of human Bacteroidales species prevents food allergy in specifically-associated germfree mice and in therapeutically treated conventional Il4raF709 mice. FIG. 21A shows a schematic representation of specific-association studies in germfree mice with the Bacteroidales microbial consortium versus gnotobiotic controls. FIG. 21A also shows core body temperature changes in GF Il4raF709 mice that were uncolonized or reconstituted with the Bacteroidales consortium, then either sham- (PBS) or OVA/SEB-sensitized and challenged with OVA. FIG. 21B shows total and OVA-specific serum IgE concentrations post-OVA challenge. FIG. 21C shows serum MMCP-1 concentrations post OVA challenge. FIG. 21D shows frequencies of MLN CD4+Foxp3+, IL-4+Foxp3+ and GATA3+Foxp3+ T cells. FIG. 21E shows frequencies of Helios-NRP1-Foxp3+ T cells. For FIGS. 21A-21E: N=5 to 10 mice/group. FIG. 21F shows a schematic representation of SPF mouse studies; Abx: antibiotics. FIG. 21F also shows core body temperature changes in OVA/SEB-sensitized and OVA-challenged Il4raF709 mice that were either untreated or treated with the Bacteroidales consortium. FIG. 21G shows total and OVA-specific IgE responses and serum MMCP-1 concentrations post OVA challenge. FIG. 21H shows frequencies of CD4+Foxp3+, IL-4+Foxp3+GATA3+Foxp3+, and FIG. 21I shows Helios-NRP1-Foxp3+ T cells in the MLN. For f-i: N=5-8 mice/group. *P<0.05, **P<0.01,***P<0.001, ****P<0.0001 by one-way ANOVA with Dunnett post hoc analysis. For core body temperature measurements ****P<0.0001 by repeat measures two-way ANOVA.

FIG. 22A-22D demonstrates that treatment with the Clostridiales or Bacteroidales therapeutic consortia suppress established food allergy in conventional IL4raF709 mice. FIG. 22A shows an experimental scheme (left) and core body temperature changes in OVA/SEB sensitized and OVA-challenged Il4raF709 mice treated with the respective bacterial mixes (right). FIG. 22B shows total and OVA-specific IgE. FIG. 22C shows Jejunal mast cells (arrows), mast cell counts per low powered field and serum MMCP-1 concentrations post OVA challenge. FIG. 22D shows frequencies of CD4+Foxp3+, IL-4+ CD4+Foxp3+, IL-4+ CD4+Foxp3-, and GATA3+Foxp3+ T cells in the MLN. N=5-15 mice/group. **p<0.01, ***p<0.001, ****p<0.0001 by one-way ANOVA with Dunnett post hoc analysis. For core body temperature measurements ***P<0.001 by repeat measures two-way ANOVA.

FIG. 23A-23F demonstrates Bacteroidales consortium persistence in vivo. FIG. 23A demonstrates the results of extracted stool DNA that was subject to qPCR with probes specific for each organism. No cross-reactivity was found in baseline stool from mice prior to administering the consortium (Panel A, right column). Ct values were compared against a standard curve of defined biomass of each organism spiked into conventional stool to obtain a normalized Log₁₀ CFU/g. FIG. 23B-23F shows show normalized Log₁₀ CFU/g values of stool samples for each of the organisms administered in the dose. N=6 mice group; grey dotted line indicates the sensitivity of detection or each qPCR probe. For mice falling below the sensitivity of detection, data points are placed at log₁₀=1.

FIG. 24 shows a table of bacterial species and strain designations; growth conditions for the microbial consortium; and the respective 16S rRNA sequences (SEQ ID NOs: 1-15).

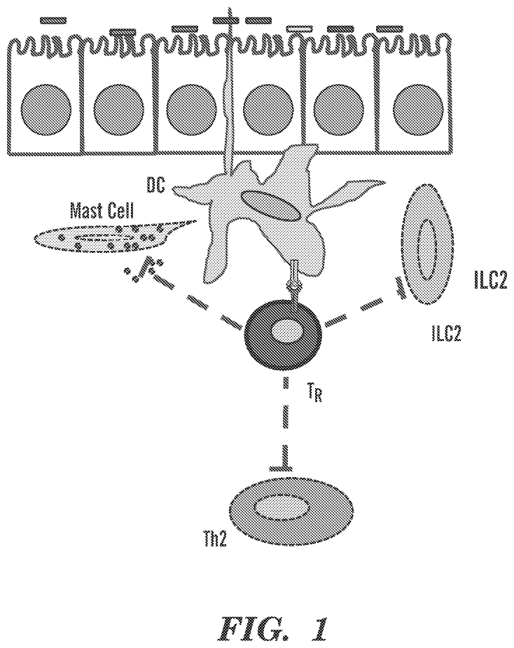

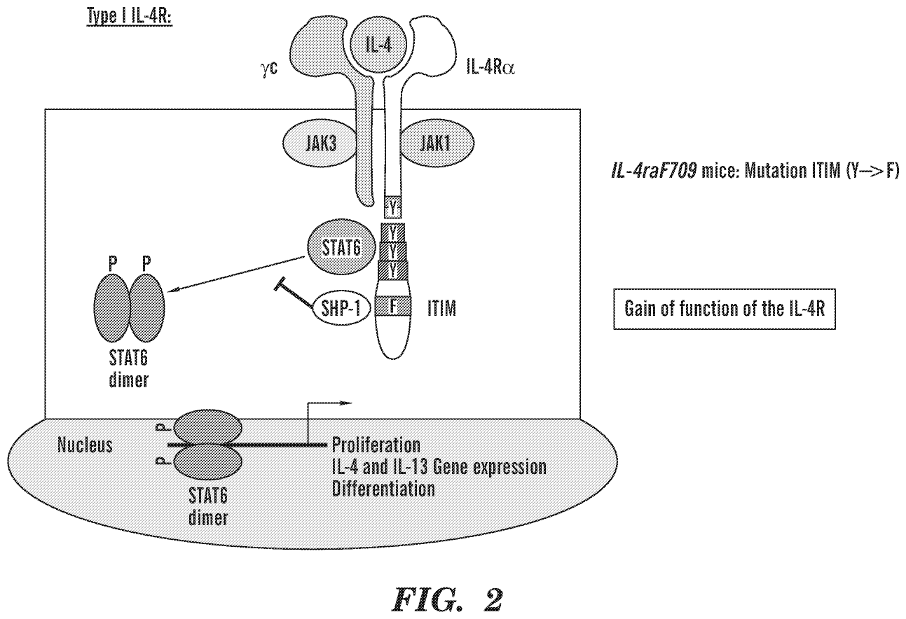

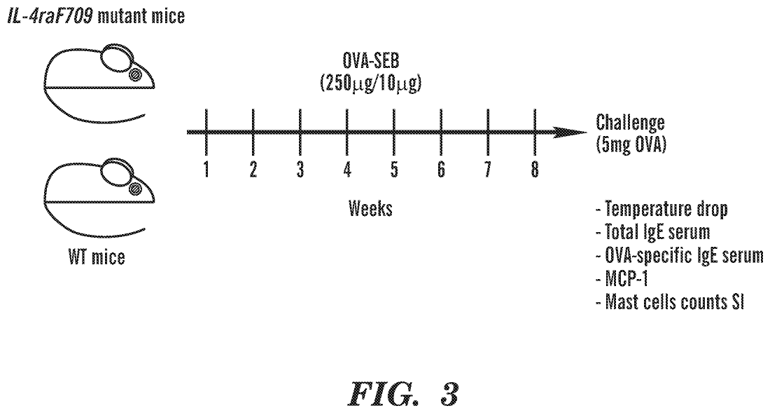

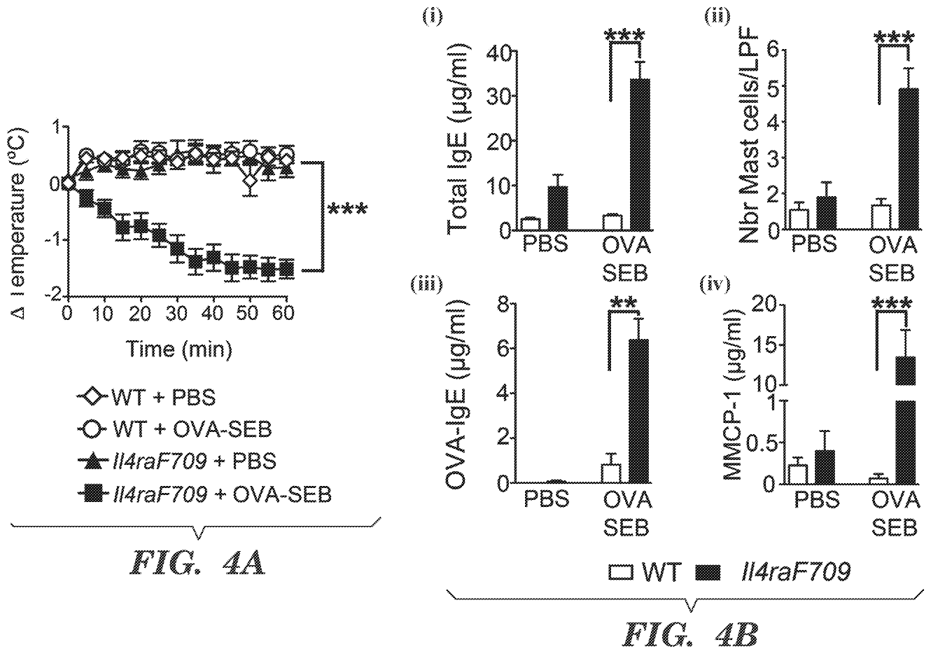

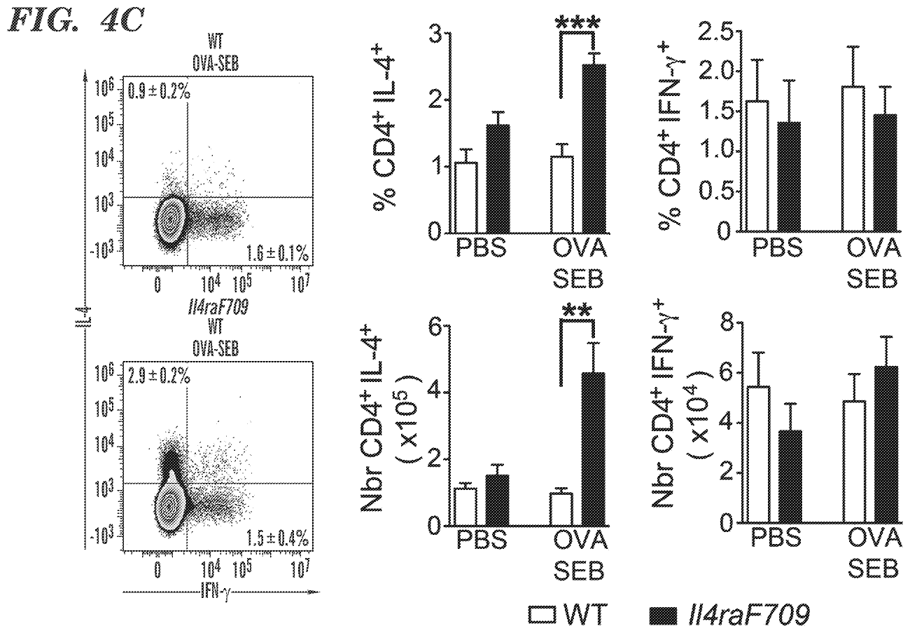

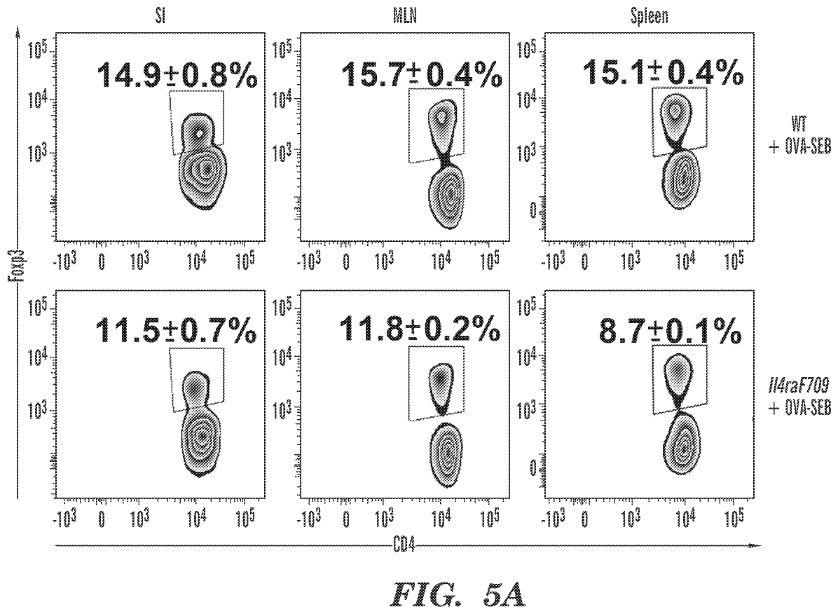

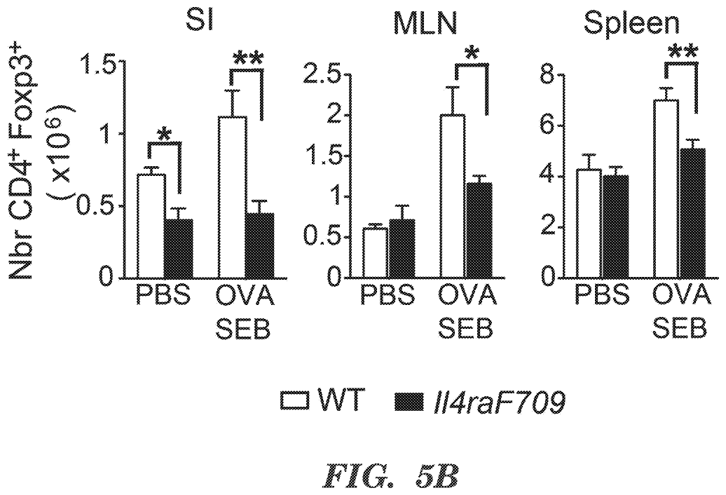

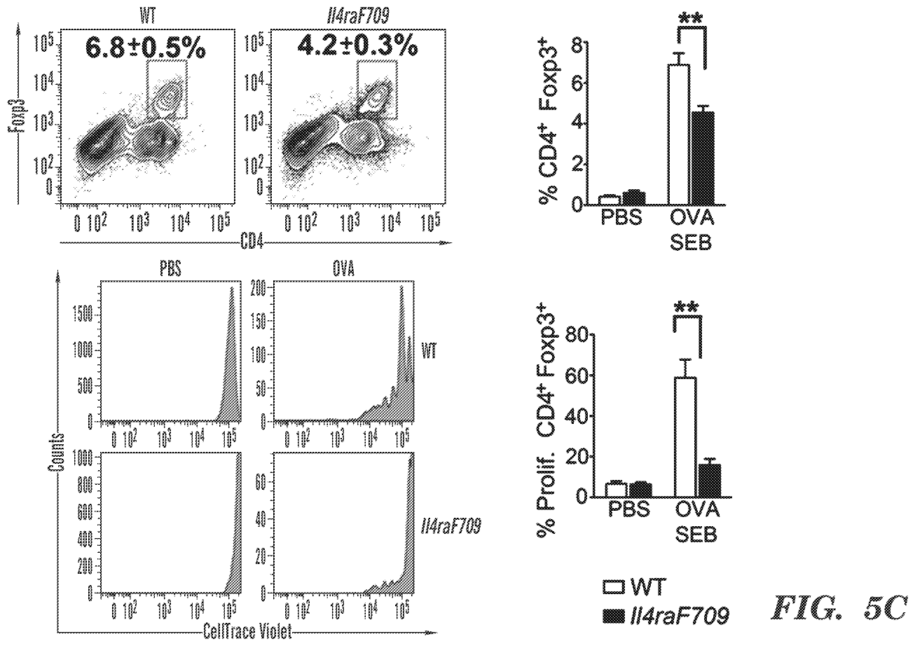

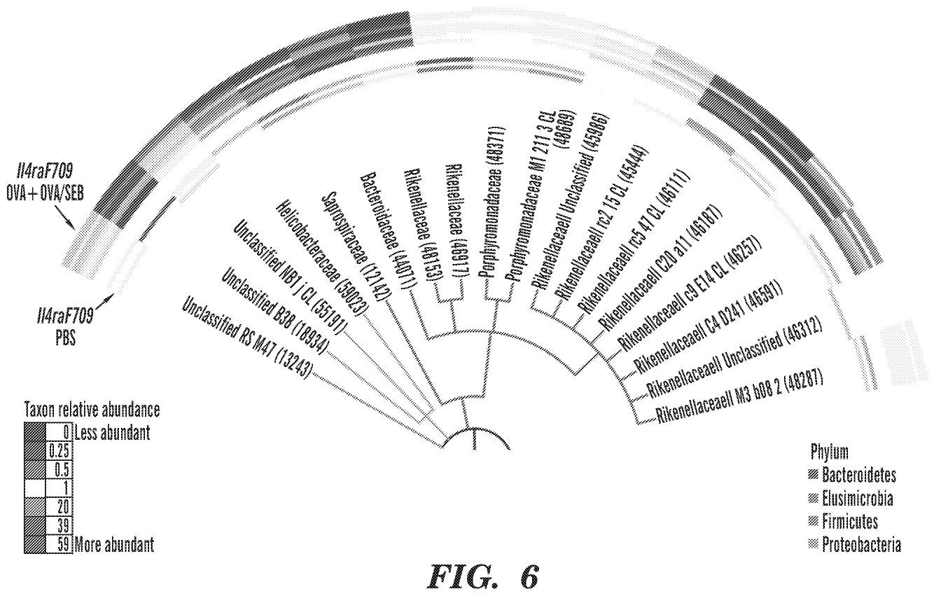

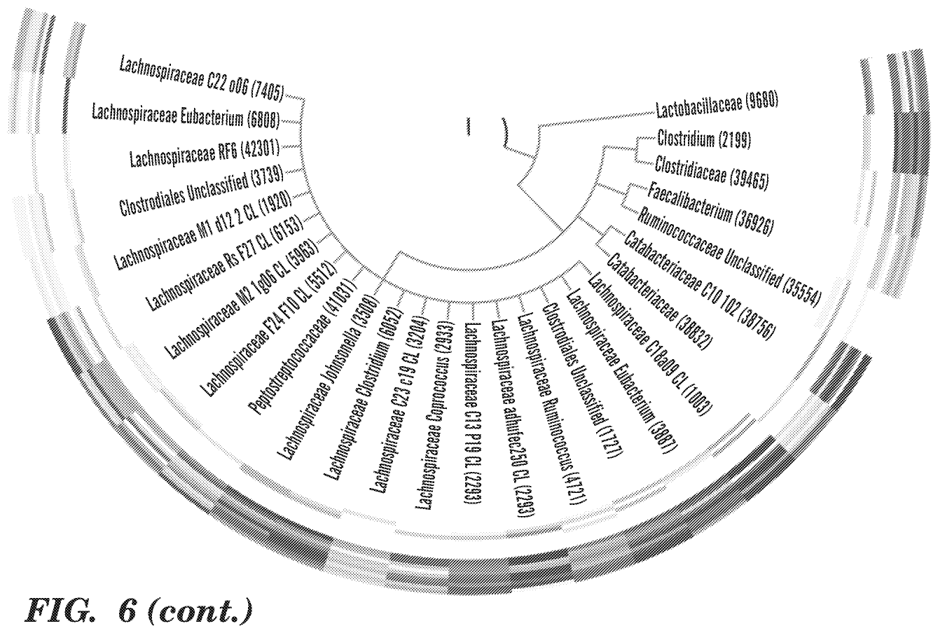

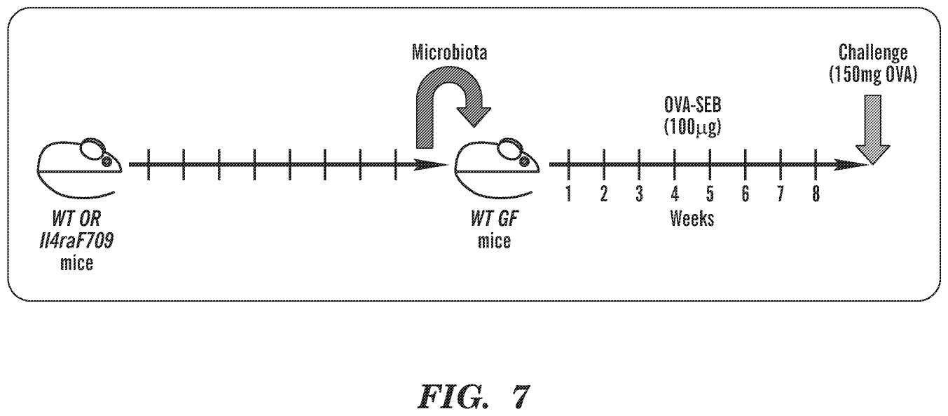

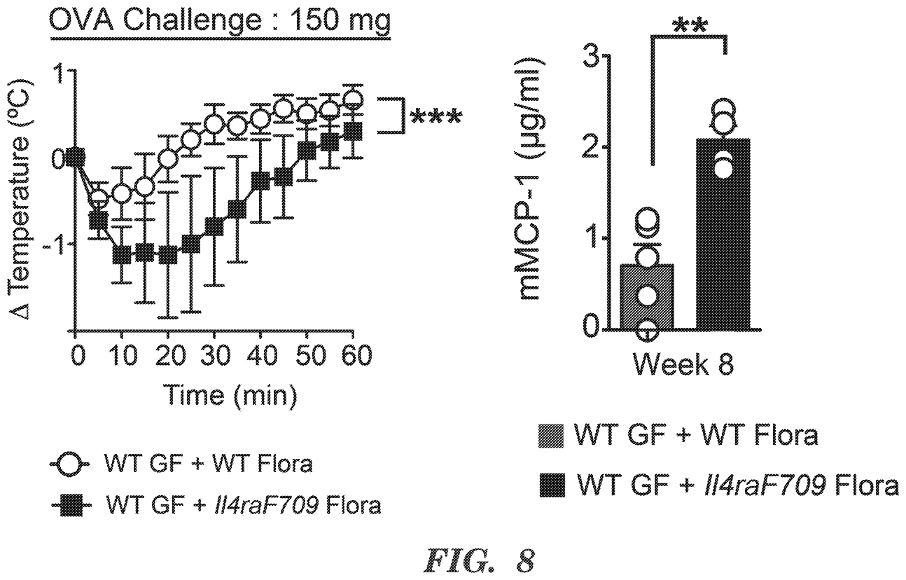

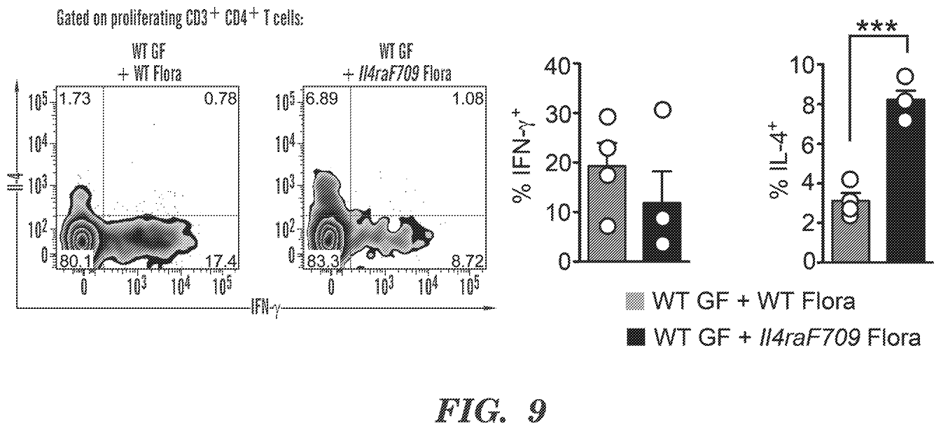

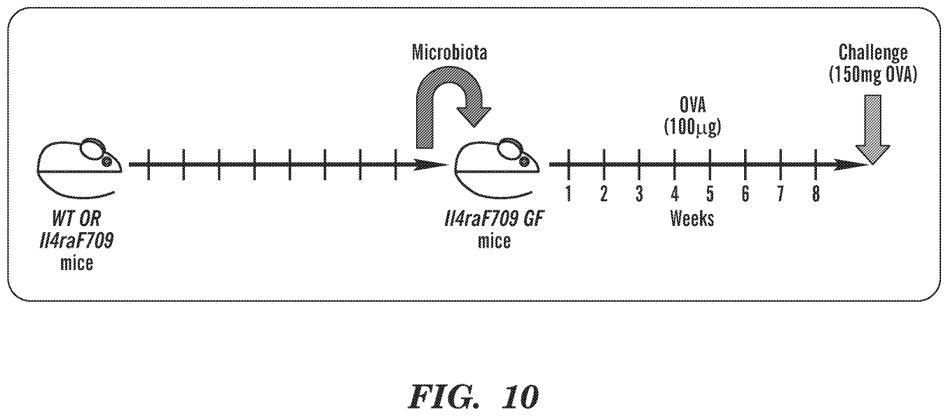

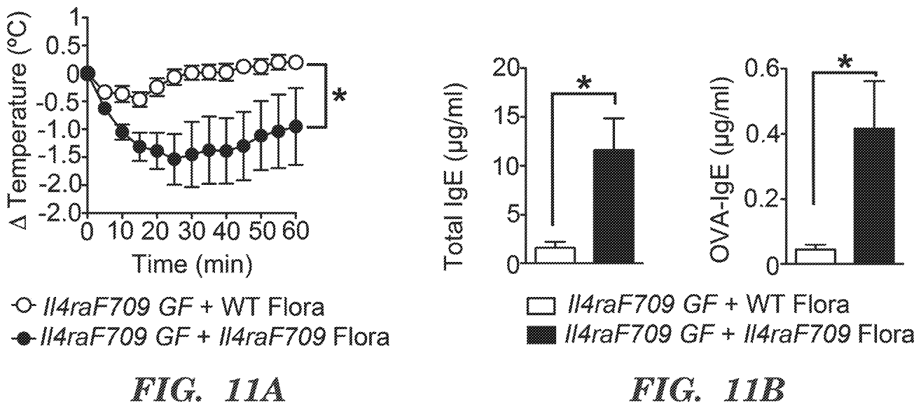

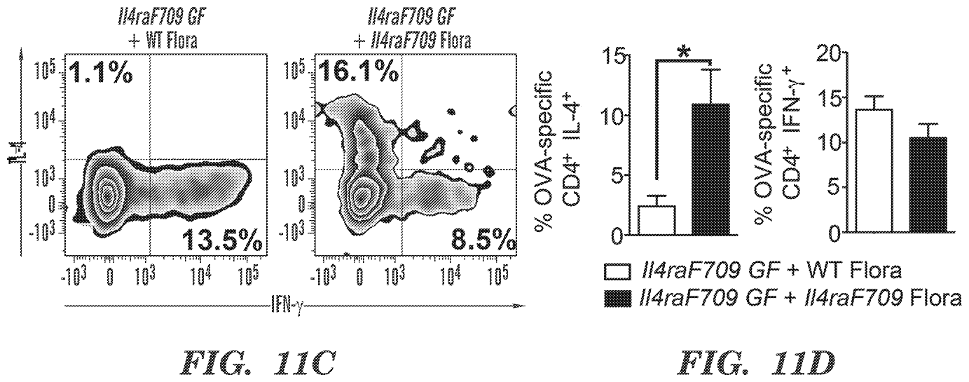

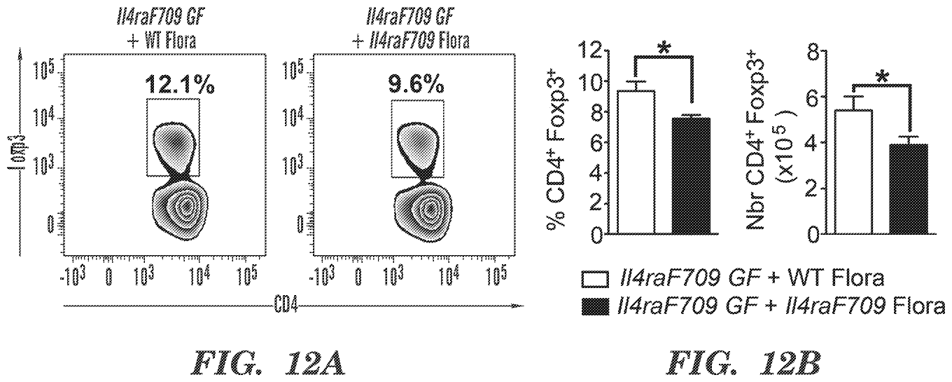

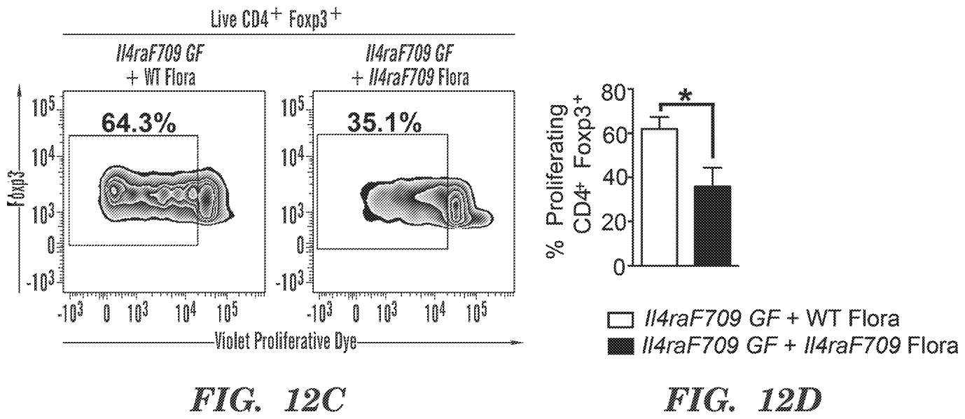

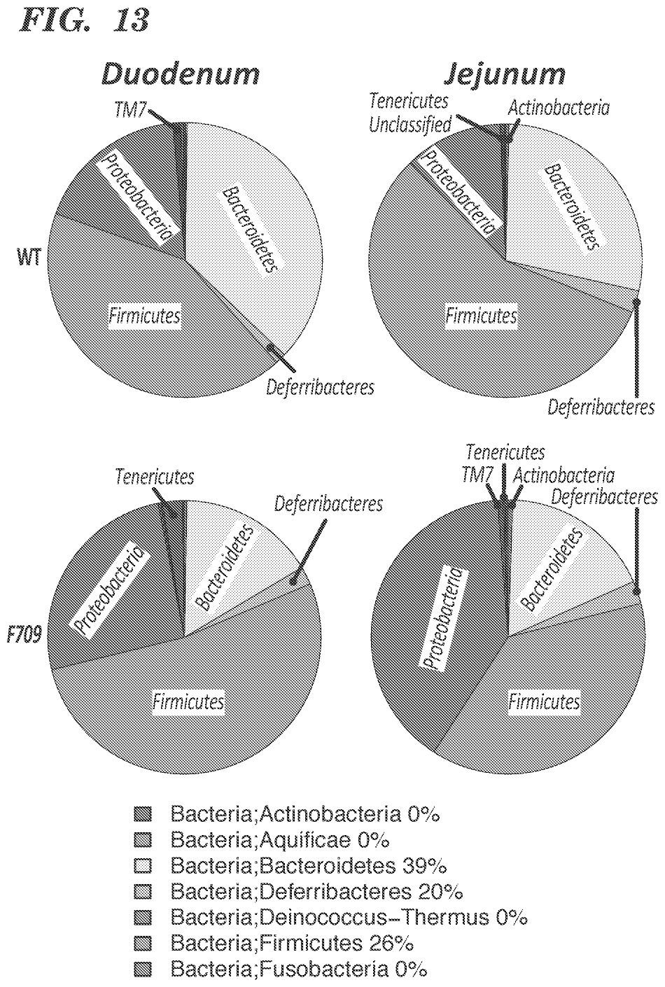

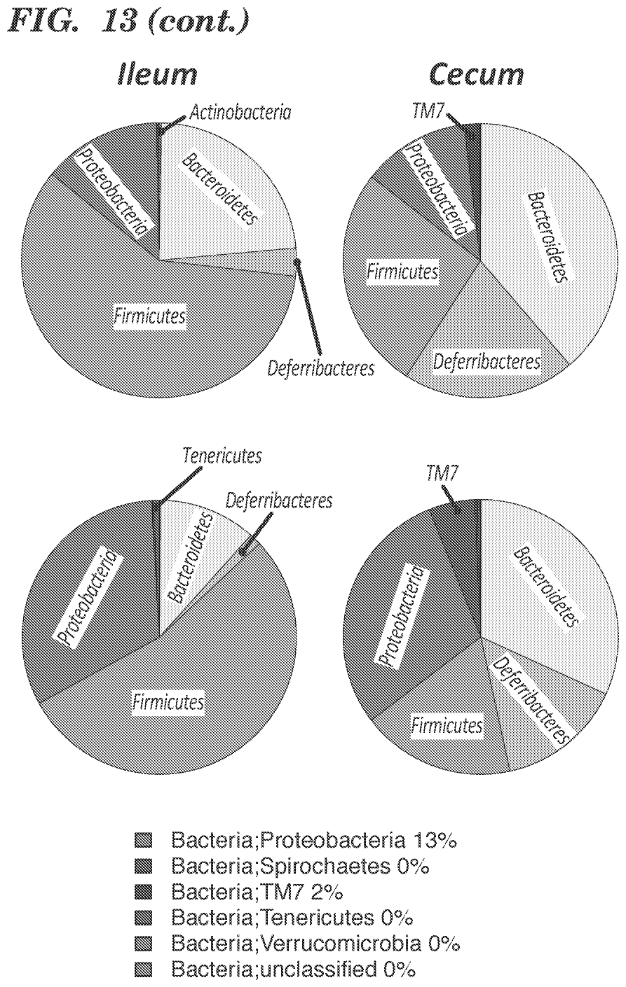

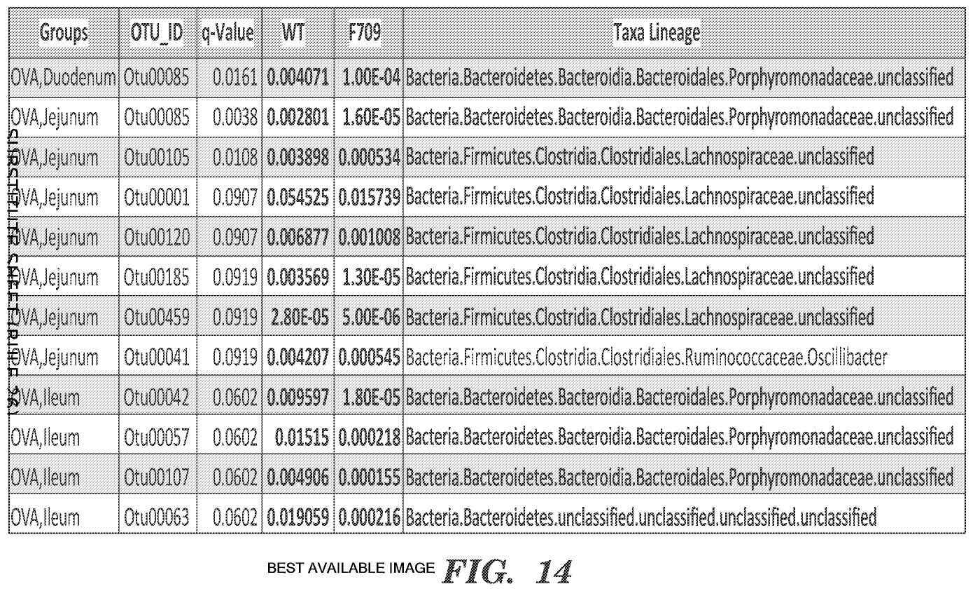

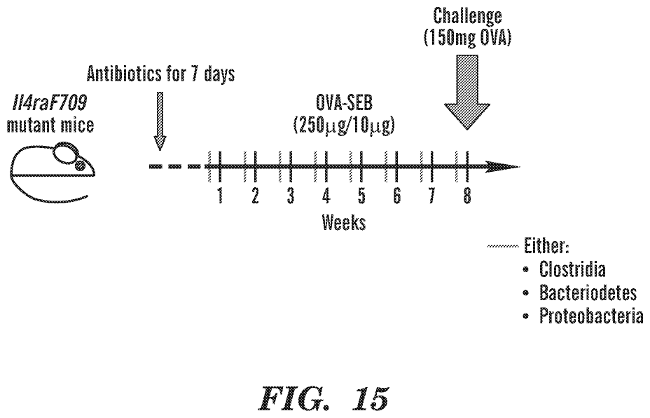

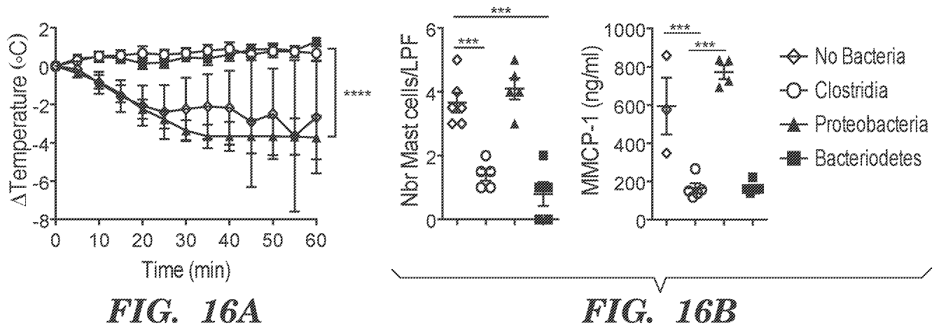

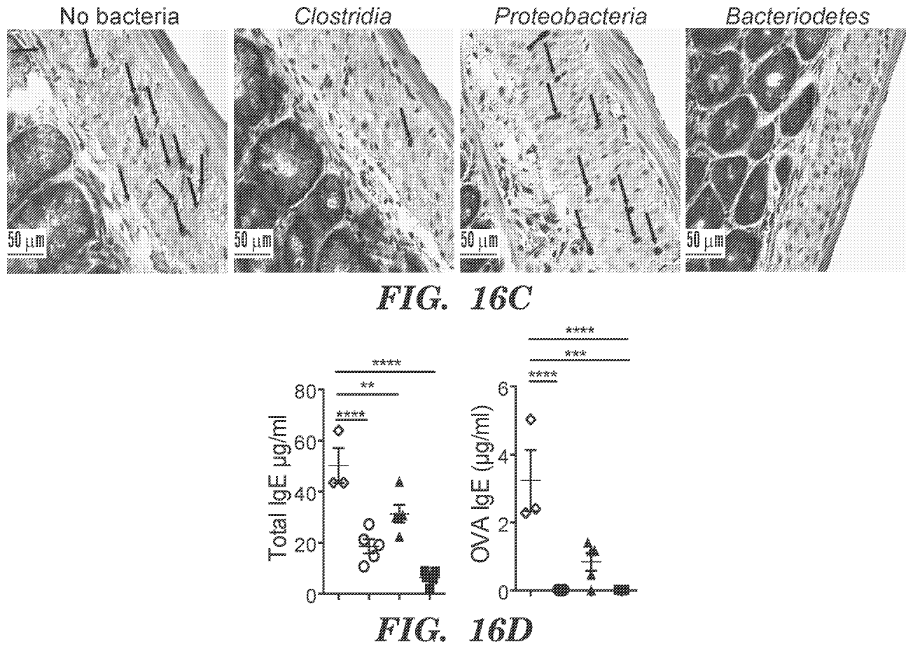

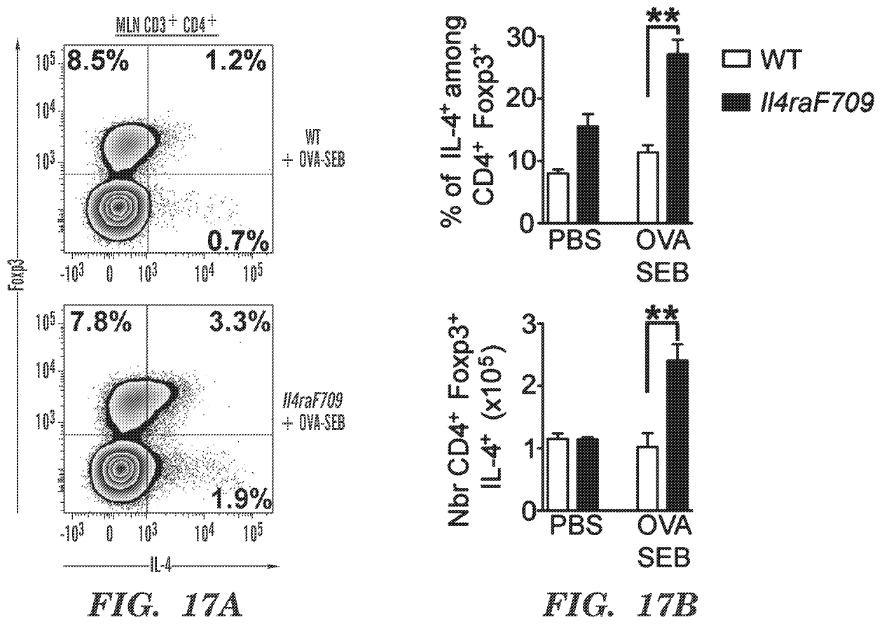

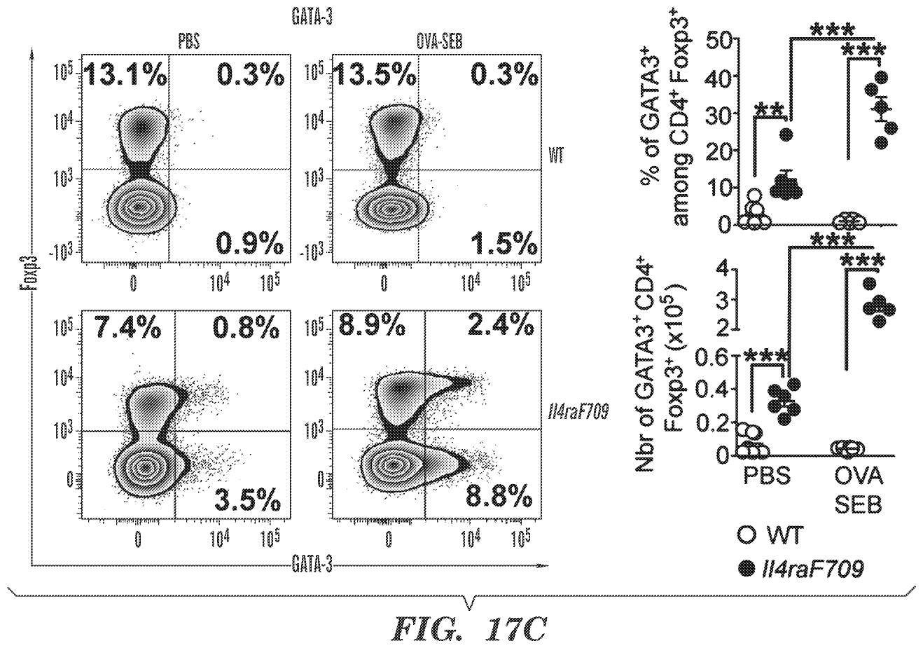

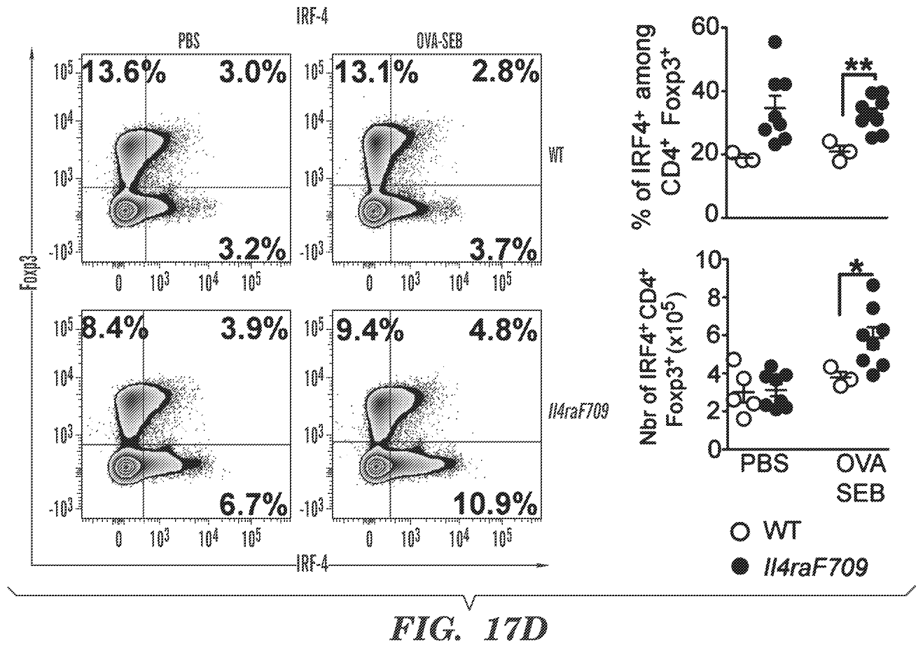

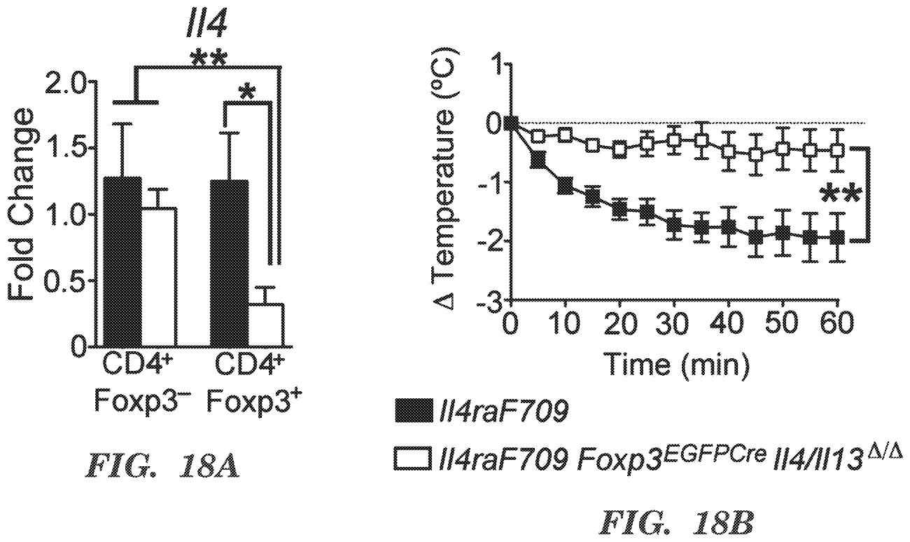

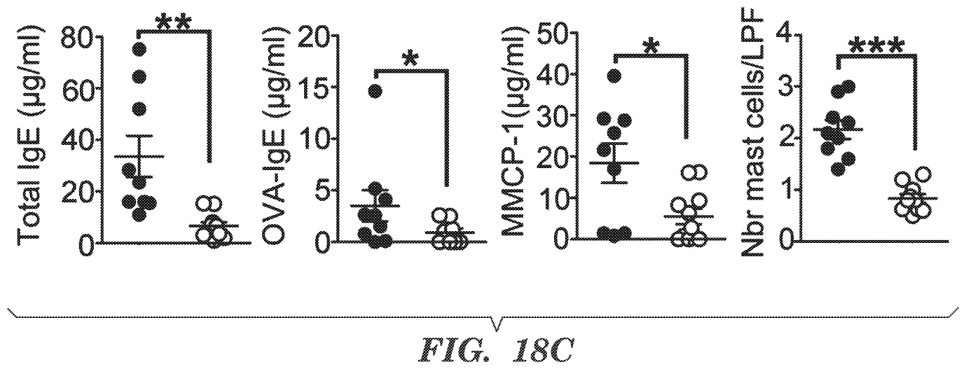

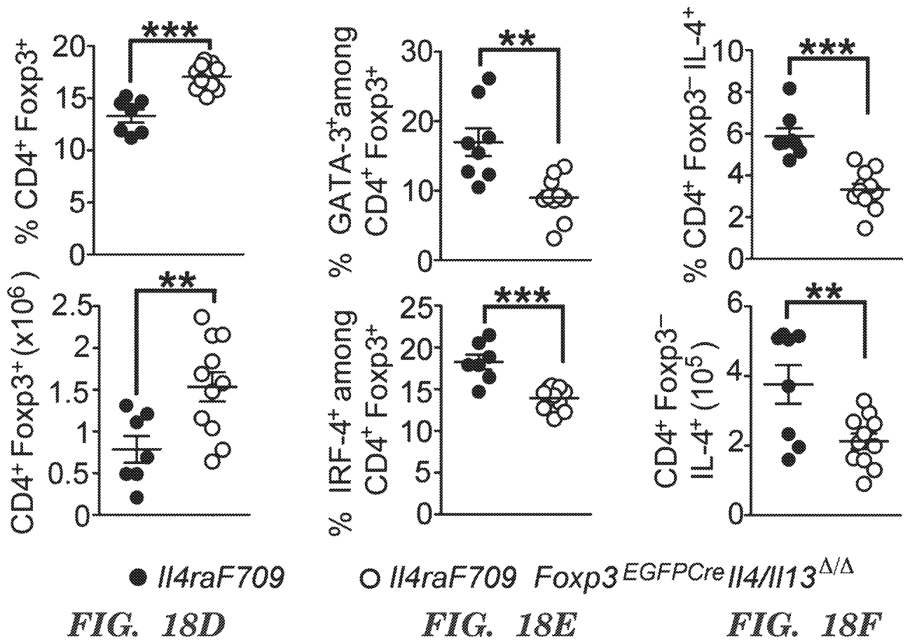

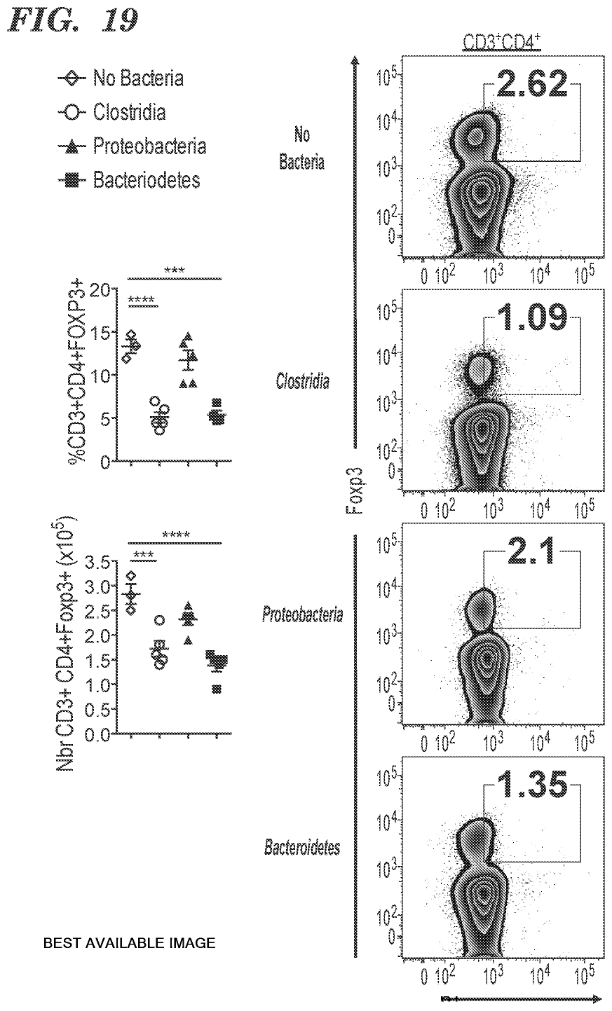

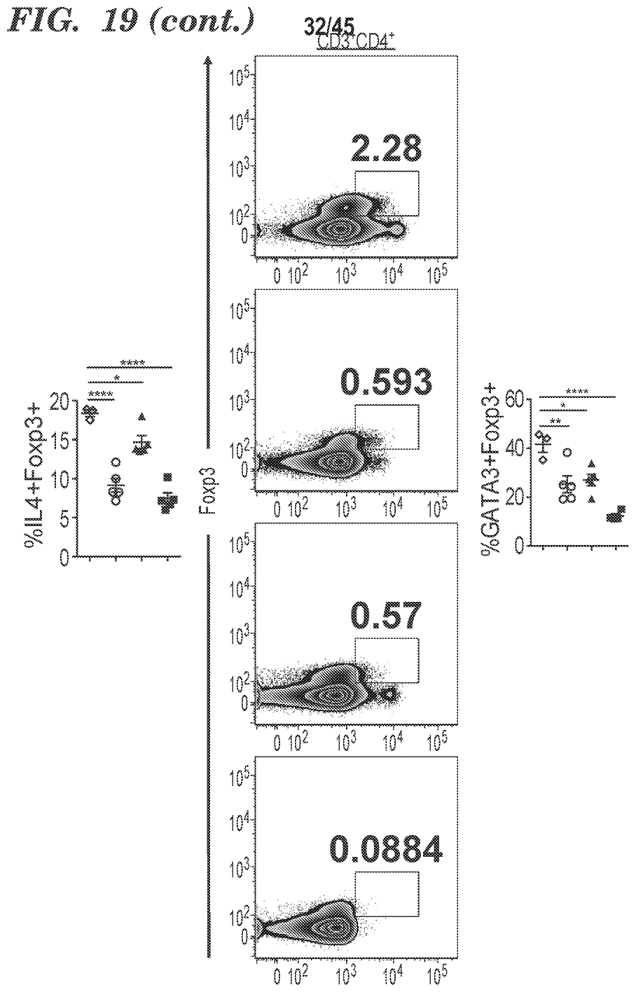

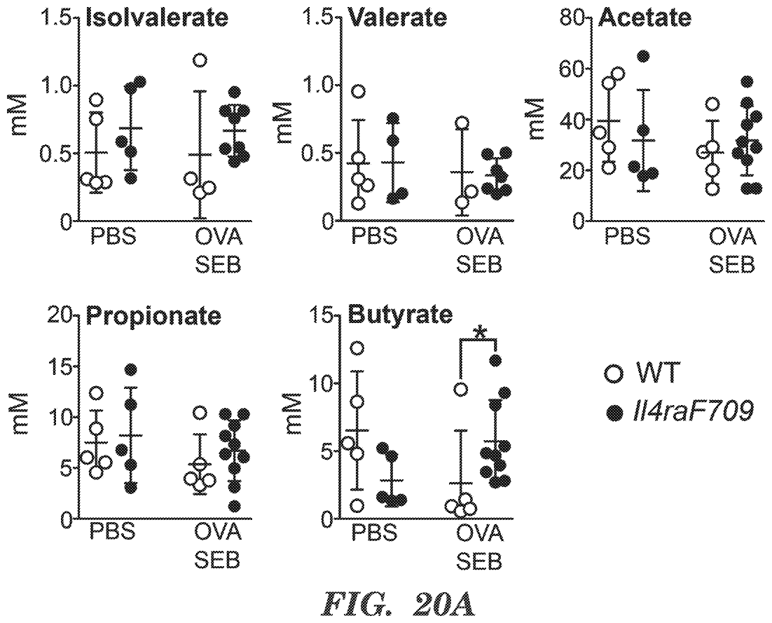

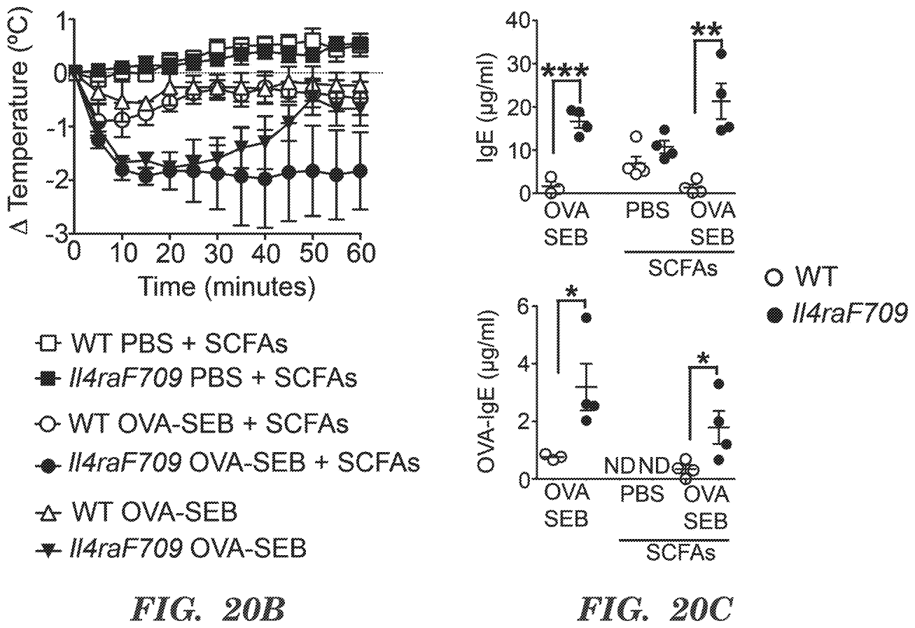

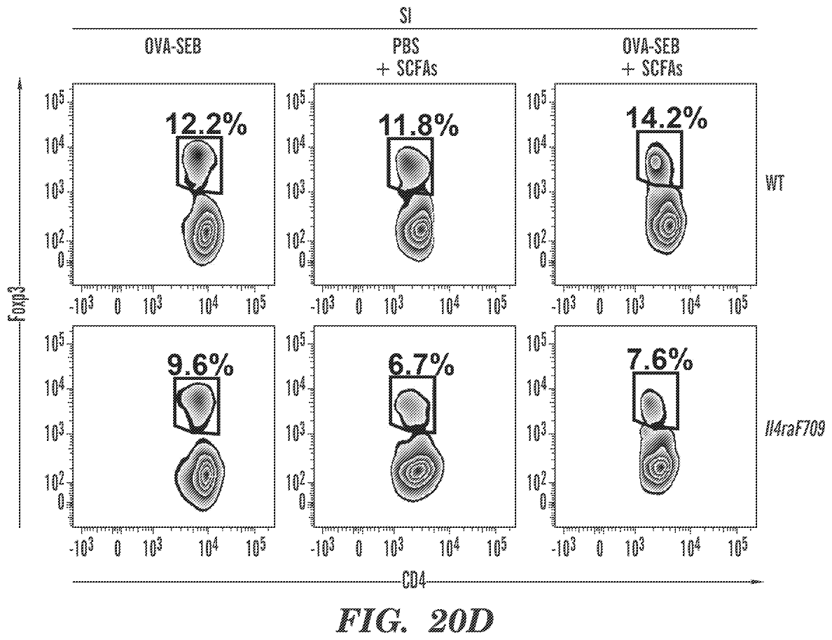

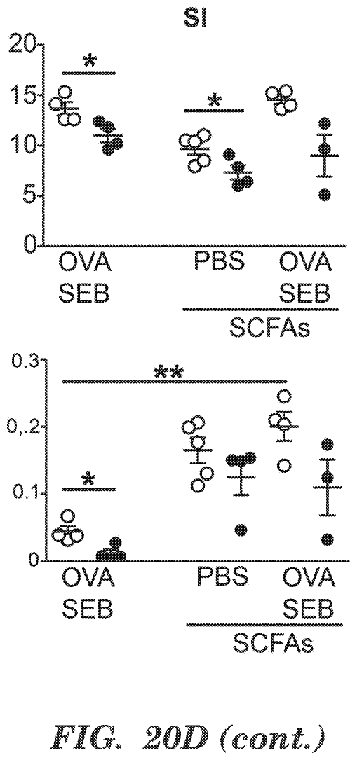

### DETAILED DESCRIPTION

Provided herein are pharmaceutical compositions, methods, microbial consortia, and microorganisms for use in the restoration of the standard microbial flora in a subject. The microorganisms described herein, or mixtures thereof, provide for the treatment and prevention of dysbiosis and associated conditions, an inflammatory disease, a metabolic disease or disorder, atopic disease or disorder, and/or an allergy. In some instances, microorganisms described herein, or mixtures thereof (e.g., microbial consortia), provide for prevention or treatment an allergy in a subject, e.g., a food allergy.

### Definitions

As used herein “preventing” or “prevention” refers to any methodology where the disease state does not occur due to the actions of the methodology (such as, for example, administration of a composition comprising a microbial consortium as described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not treated with a composition comprising a microbial consortium as described herein).

As used herein, the term “full complement of bile acid transformations” refers to the metabolism of primary bile acids to secondary bile acids. Bile acid transformations performed by gut microbes include deconjugation, deglucuronidation, oxidation of hydroxyl groups, reduction of oxo-groups to yield epimeric hydroxyl bile acids, esterification and dehydroxylation. These reactions on bile acids are the full complement of bile acid transformations as the term is used herein.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%/6, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient, allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The term “pharmaceutically acceptable carriers” excludes tissue culture media.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

It should be understood that this invention is not limited to the particular methodologies, protocols, and reagents, etc., described herein and as such can vary therefrom. The terminology used herein is for the purpose of describing particular embodiments only, and is not intended to limit the scope of the present invention, which is defined solely by the claims.

### Microbial Flora

Each individual has a personalized gut microbiota including an estimated 500 to 5000 or more species of bacteria, fungi, viruses, archaea and other microorganisms, up to 100 trillion individual organisms, that reside in the digestive tract, providing a host of useful symbiotic functions, for example, including aiding in digestion, providing nutrition for the colon, producing vitamins, regulating the immune system, assisting in defense against exogenous bacteria, modulating energy metabolism, and the production of short chain fatty acids (SCFAs), e.g., via dietary carbohydrates, including resistant starches and dietary fiber, which are substrates for fermentation that produce SCFAs, primarily acetate, propionate, succinate, butyrate, 1,2 propanediol or 1,3 propanediol as end products.

An imbalance in the microbial flora found in and on the human body is known to be associated with a variety of disease states. For example, obesity in both humans and experimental mouse models is associated with alterations in the intestinal microbiota that appear to be pathogenic. In settings of ‘dysbiosis’ or disrupted symbiosis, microbiota functions that can be lost or deranged, resulting in increased susceptibility to pathogens, include altered metabolic profiles, or induction of proinflammatory signals that can result in local or systemic inflammation or autoimmunity. In addition, in asthmatic subjects, both the bacterial burden and bacterial diversity were significantly higher as compared to control subjects, which were also correlated with bronchial hyper-responsiveness. Thus, the intestinal microbiota plays a significant role in the pathogenesis of many diseases and disorders, including a variety of pathogenic infections of the gut. For instance, patients become more susceptible to pathogenic infections when the normal intestinal microbiota has been disturbed due to use of broad-spectrum antibiotics. Many of these diseases and disorders are chronic conditions that significantly decrease a patient's quality of life and can be ultimately fatal.

### Microbial Consortia

In one embodiment, a microbial consortium of isolated bacteria useful in the compositions and methods described herein comprises two to twenty, two to nineteen, two to eighteen, two to seventeen, two to sixteen, two to fifteen, two to fourteen, two to thirteen, two to twelve, two to eleven, two to ten, two to nine, two to eight, two to seven, two to six, two to five, two to four, or two to three species of viable gut bacteria. In another embodiment, the microbial consortium of isolated bacteria comprises no more than forty species, no more than 35 species, no more than 30 species, or no more than 25 species. In another embodiment, the microbial consortium comprises two to twenty-one species, two to twenty-two species, two to twenty-three species, two to twenty-four species, two to twenty-five species, two to twenty-six species, two to twenty-seven species, two to twenty-eight species, two to twenty-nine species, two to thirty species, two to thirty-one species, two to thirty-two species, two to thirty-three species, two to thirty-four species, two to thirty-five species, two to thirty-six species, two to thirty-seven species, two to thirty-eight species, tow to thirty-nine species or two to forty species

In some embodiments, a microbial consortium comprises at least 2, at least 3, at least 4, at least 5, at least 6, at least 7, at least 8, at least 9, at least 10, at least 11, at least 12 or more different viable, bacterial species, e.g., 15 or more, 20 or more, 25 or more, 30 or more, or even 40 species. In another embodiment, the microbial consortium comprises 12 or less, 11 or less, 10 or less, 9 or less, 8 or less, 7 or less, 6 or less, 5 or less, 4 or less, or 3 or less different viable bacterial species. Also contemplated are consortia of 2 to 40 species, 4 to 30 species, 4 to 25 species, 4 to 20 species, 4 to 15 species, 4 to 11 species, 5 to 40 species, 5 to 30 species, 5 to 25 species, 5 to 20 species, 5 to 15 species, 5 to 11 species, 6 to 40 species, 6 to 30 species, 6 to 25 species, 6 to 20 species, 6 to 15 species, 6 to 11 species, 7 to 40 species, 7 to 30 species, 7 to 25 species, 7 to 20 species, 7 to 15 species, 7 to 11 species, 8 to 40 species, 8 to 30 species, 8 to 25 species, 8 to 20 species, 8 to 15 species, 8 to 11 species, 9 to 40 species, 9 to 30 species, 9 to 25 species, 9 to 20 species, 9 to 15 species, 9 to 11 species, 10 to 40 species, 10 to 30 species, 10 to 25 species, 10 to 20 species, 10 to 15 species, or 10 to 11 species.

In one embodiment, a therapeutic microbial consortium for the treatment or prevention of an indication as described herein comprises at least two bacterial strain(s) of viable gut bacteria selected from the group consisting of: Bacteroides fragilis, Bacteroides ovatus, Bacteroides vulgatus, Parabacteroides distasonis, and Prevotella melaninogenica. In another embodiment, the therapeutic microbial consortium for the treatment or prevention of an indication as described herein further comprises one or more, two or more, three or more, four or more, five or more, or all six of the species including: Clostridium ramosum, Clostridium scindens, Clostridium hiranonsis, Clostridium bifermentans, Clostridium leptum, and Clostridium sardiniensis.

In another embodiment, a therapeutic microbial consortium comprises at least three bacterial strain(s) of viable gut bacteria selected from the group consisting of: Bacteroides fragilis, Bacteroides ovatus, Bacteroides vulgatus, Parabacteroides distasonis, and Prevotella melaninogenica. In another embodiment, the therapeutic microbial consortium for the treatment or prevention of an indication as described herein further comprises one or more, two or more, three or more, four or more, five or more, or all six of the species including: Clostridium ramosum, Clostridium scindens, Clostridium hiranonsis, Clostridium bifermentans, Clostridium leptum, and Clostridium sardiniensis.

In another embodiment, a therapeutic microbial consortium comprises at least four bacterial strain(s) of viable gut bacteria selected from the group consisting of: Bacteroides fragilis, Bacteroides ovatus, Bacteroides vulgatus, Parabacteroides distasonis, and Prevorella melaninogenica. In another embodiment, the therapeutic microbial consortium for the treatment or prevention of an indication as described herein further comprises one or more, two or more, three or more, four or more, five or more, or all six of the species including: Clostridium ramosum, Clostridium scindens, Clostridium hiranonsis, Clostridium bifermentans, Clostridium leptum, and Clostridium sardiniensis.

In another embodiment, a therapeutic microbial consortium comprises each of the bacterial strain(s) of viable gut bacteria selected from the group consisting of: Bacteroides fragilis, Bacteroides ovatus, Bacteroides vulgatus, Parabacteroides distasonis, and Prevotella melaninogenica In another embodiment, the therapeutic microbial consortium for the treatment or prevention of an indication as described herein further comprises one or more, two or more, three or more, four or more, five or more, or all six of the species including: Clostridium ramosum, Clostridium scindens, Clostridium hiranonsis, Clostridium bifermentans, Clostridium leptum, and Clostridium sardiniensis.

In another embodiment, the microbial consortium comprises at least two species of viable gut bacteria are selected from the group consisting of: Bacteroides fragilis, Bacteroides ovatus, Bacteroides vulgatus, Parabacteroides distasonis, Prevotella melaninogenica, and the microbial consortium further comprises one or more, two or more, three or more, four or more, five or more, or all six of Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus. In another embodiment, the microbial consortium further comprises at least one of the group consisting of: Bacteroides vulgatus, Parabacteroides distasonis, and Prevotella melaninogenica.

In another embodiment, the microbial consortium comprises Bacteroides fragilis and Bacteroides ovatus. In another embodiment, the microbial consortium further comprises at least two of the group consisting of: Bacteroides vulgatus, Parabacteroides distasonis, and Prevotella melaninogenica.

In another embodiment, the microbial consortium comprises Bacteroides fragilis and Bacteroides ovatus, and the microbial consortium further comprises each of the group consisting of: Bacteroides vulgatus, Parabacteroides distasonis, and Prevotella melaninogenica.

In another embodiment, the microbial consortium comprises Bacteroides fragilis and Bacteroides ovatus, and at least one of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and at least two of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and at least three of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and at least four of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and at least five of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides ovatus, and each of the group consisting of Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides vulgatus and at least one of the group consisting of: Bacteroides ovatus, Parabacteroides distasonis, and Prevotella melaninogenica.

In another embodiment, the microbial consortium comprises Bacteroides fragilis is and Bacteroides vulgatus and at least two of the group consisting of: Bacteroides ovatus, Parabacteroides distasonis, and Prevotella melaninogenica.

In another embodiment, the microbial consortium comprises Bacteroides fragilis and Bacteroides vulgatus and further comprises Bacteroides ovatus, Parabacteroides distasonis, and Prevotella melaninogenica.

In another embodiment, the microbial consortium comprises Bacteroides fragilis and Bacteroides vulgatus and at least one of the group consisting of: Clostridium ramosum, Clostridium scindens, Clostridium hiranonsis, Clostridium bifermentans, Clostridium leptum, and Clostridium sardiniensis.

In another embodiment, the microbial consortium further comprises Bacteroides fragilis and Bacteroides vulgatus and at least two of the group consisting of: Clostridium ramosum, Clostridium scindens, Clostridium hiranonsis, Clostridium bifermentans, Clostridium leptum, and Clostridium sardiniensis.

In another embodiment, the microbial consortium further comprises Bacteroides fragilis and Bacteroides vulgatus and at least three of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides vulgatus and at least four of the group consisting of: Clostridium ramosum, Clostridium scindens, Clostridium hiranonsis, Clostridium bifermentans, Clostridium leptum, and Clostridium sardiniensis.

In another embodiment, the microbial consortium further comprises Bacteroides fragilis and Bacteroides vulgatus and at least five of the group consisting of: Clostridium ramosum, Clostridium scindens, Clostridium hiranonsis, Clostridium bifermentans, Clostridium leptum, and Clostridium sardiniensis.

In another embodiment, the microbial consortium comprises Bacteroides fragilis and Bacteroides vulgatus and further comprises Clostridium ramosum, Clostridium scindens, Clostridium hiranonsis, Clostridium bifermentans, Clostridium leptum, and Clostridium sardiniensis.

Bacterial species or bacterial strains in consortia described herein are not pathogenic in the human gut.

In one embodiment, the species of viable gut bacteria do not include Escherichia coli, Klebsiella pneumoniae, Proteus mirabilis, Enterobacter cloacae, Bilophila wadsworthia, Alistipes onderdonkii, Desulfovibrio species, Lactobacillus johnsoni, and Parasutterella excrementihominis.

In another embodiment, the consortium does not comprise bacteria of the Genera Bilophila, Enterobacter, Escherichia, Klebsiella, Proteus, Alistipes, Desulfovibrio, Blautia, or Parasutterella.

In another embodiment, the consortium does not comprise bacteria of the Families Desulfovibrionaceae, Enterobacteriaceae, Rikenellaceae, and Sutterellaceae.

In another embodiment, the consortium does not comprise bacteria of the Families Lactobacillaceae or Enterbacteriaceae.

In another embodiment, the consortium does not comprise bacteria of the Order Burkholdales, Desulfovibrionales, or Enterobacteriales.

Metabolic Features: Various features of gut microbes are beneficial for protection from or therapy for allergy, including food allergy. In the following, features and corresponding functions contemplated to render particular species or taxa of gut microbes well-suited for a protective or therapeutic microbial consortium as described herein are described. In practice, a consortium comprising four or more, e.g., five or more, six or more, seven or more, eight or more, nine or more or ten or more of these features and corresponding functions is considered a likely candidate for protection or therapy for food allergy.

In some embodiments, the microbial consortium comprises one or more types of microbes capable of producing butyrate in a mammalian subject. Butyrate-producing microbes can be identified experimentally, e.g., by NMR or gas chromatography analyses of microbial products or colorimetric assays (Rose I A. 1955. Methods Enzymol. 1: 591-5). Butyrate-producing microbes can also be identified computationally, e.g., by the identification of one or more enzymes involved in butyrate synthesis. Non-limiting examples of enzymes found in butyrate-producing microbes include butyrate kinase, phosphotransbutyrylase, and butyryl CoA:acetate CoA transferase (Louis P., et al. 2004. J Bact. 186(7): 2099-2106). Butyrate-producing species include, but are not limited to, Clostridium sardiniensis, Clostridium hiranonsis, Faecalibacterium prausnitzii, Butyrivibrio spp., Eubacterium rectale, and Roseburia intestinalis.

In some embodiments, a pharmaceutical composition comprises one or more types of microbes or bacterial species, wherein the at least two types of microbes are capable of producing butyrate in a mammalian subject. In other embodiments, the composition comprises two or more types of microbes, that cooperate (i.e., cross-feed) to produce an immunomodulatory short chain fatty acid (SCFA) (e.g., butyrate) in a mammalian subject. In one embodiment, the composition comprises at least one type of microbe (e.g., Bifidobacterium spp., Bacteroides vulgatus, Bacteroides fragilis or Clostridium ramosum) capable of metabolizing a prebiotic, including but not limited to, inulin, inulin-type fructans, fucose-containing glycoconjugates including the H1, H2, Lewis A, B, X, or Y antigens, or oligofructose, such that the resulting metabolic product can be converted by a second type of microbe (e.g., a butyrate-producing microbe such as Roseburia spp.) to an immunomodulatory SCFA such as butyrate (Falony G., et al. 2006 Appl. Environ. Microbiol. 72(12): 7835-7841). In other aspects, the composition can comprise at least one acetate-consuming, butyrate-producing microbe (e.g., Faecalibacterium prausnitzii or Roseburia intestinalis).

In some embodiments, the composition comprises one or more types of microbe capable of producing propionate and/or succinate in a mammalian subject, optionally further comprising a prebiotic or substrate appropriate for propionate and/or succinate biosynthesis. Examples of prebiotics or substrates used for the production of propionate include, but are not limited to, L-rhamnose, D-tagalose, resistant starch, inulin, polydextrose, arabinoxylans, arabinoxylan oligosaccharides, mannooligosaccharides, and laminarans (Hosseini E., et al. 2011. Nutrition Reviews 69(5): 245-258). Propionate-producing microbes can be identified experimentally, such as by NMR or gas chromatography analyses of microbial products or colorimetric assays (Rose I A. 1955. Methods Enzymol. 1: 591-5). Propionate-producing microbes can also be identified computationally, such as by the identification of one or more enzymes involved in propionate synthesis Non-limiting examples of enzymes found in propionate-producing microbes include enzymes of the succinate pathway, including but not limited to phosphoenylpyruvate carboxykinase, pyruvate kinase, pyruvate carboxylase, malate dehydrogenase, fumarate hydratase, succinate dehydrogenase, succinyl CoA synthetase, methylmalonyl CoA decarboxylase, and propionate CoA transferase, as well as enzymes of the acrylate pathway, including but not limited to L-lactate dehydrogenase, propionate CoA transferase, lactoyl CoA dehydratase, acyl CoA dehydrogenase, phosphate acetyltransferase, and propionate kinase. For example, microbes that utilize the succinate pathway include certain species of the Bacteroides genus, such as Bacteroides fragilis, Clostridium sardiniensis and Clostridium hiranonsis. In one embodiment, the propionate-producing species is Bacteroides fragilis, Bacteroides thetaiotaomicron, or Bacteroides ovatus. In one embodiment, the succinate-producing species is Bacteroides fragilis, Bacteroides thetaiotaomicron, Bacteroides vulgatus, or Bacteroides ovatus.

Functional methods to define species that produce butyrate, propionate and/or succinate includes analysis of short-chain fatty acid (SCFA) production using gas-chromatography/liquid chromatography (GC/LC) to identify propionate, butyrate, and/or succinate or mass spectroscopy based methods to detect these SCFA, as well as 1,2-propanediol, and 1,3-propanediol. Studies can be performed in cultured supernatants from colonized gnotobiotic mice and from conventional patients and/or animal samples.

Additional methods for identifying species that produce butyrate comprise those species expressing butyryl-CoA:acetate CoA transferases (But genes) or butyrate kinases (Buk genes) for production of butyrate from anaerobic fermentation of sugars. In another embodiment, organisms producing butyrate (from amino acids such as lysine, glutarate or 4-aminobutyrate pathways) express enzymes including e.g., L2Hgdh, 2-hydroxyglutarate dehydrogenase; Get, glutaconate CoA transferase (α, β subunits); HgCoAd, 2-hydroxy-glutaryl-CoA dehydrogenase (α, β, γ subunits); Gcd, glutaconyl-CoA decarboxylase (α, β subunits); Thl, thiolase; hbd, β-hydroxybutyryl-CoA dehydrogenase; Cro, crotonase; Bcd, butyryl-CoA dehydrogenase (including electron transfer protein α, β subunits); KamA, lysine-2,3-aminomutase; KamD,E, β-lysine-5,6-aminomutase (a, P subunits); Kdd, 3,5-diaminohexanoate dehydrogenase; Kce, 3-keto-5-aminohexanoate cleavage enzyme; Kal, 3-aminobutyryl-CoA ammonia lyase; AbfH, 4-hydroxybutyrate dehydrogenase; AbfD, 4-hydroxybutyryl-CoA dehydratase; Isom, vinylacetyl-CoA 3,2-isomerase (same protein as AbfD): 4Hbt, butyryl-CoA:4-hydroxybutyrate CoA transferase; But, butyryl-CoA:acctate CoA transferase; Ato, butyryl-CoA:acetoacetate CoA transferase (α, β subunits); Ptb, phosphate butyryltransferase; Buk, and butyrate kinase (see e.g., Vital et al. mBIO 5(2):e00889-14).

In some embodiments, a microbial consortium comprises at least one bacterial species that produces compounds capable of stimulating the aryl hydrocarbon (AhR) receptor in gut epithelial cells, antigen-presenting cells and/or T cells. Without wishing to be bound by theory, stimulation of the AhR receptor can aid in the development of regulatory T cell processes that can prevent and/or treat food allergy. Some non-limiting examples of compounds that stimulate host aryl hydrocarbon receptor pathways include (i) indole, (ii) intermediates from microbial synthesis of indole, tryptophan, tyrosine and histidine, (iii) microbial synthesis of flavonoids, phenazines and/or quinones or (iv) compounds or intermediates of metabolism of host ingested flavonoids, phenazines and/or quinones. In one example, a viable, culturable, anaerobic gut bacterial strain produces aryl hydrocarbon receptor agonists sufficient to stimulate host aryl hydrocarbon receptor pathways comprises at least one gene associated with the synthesis of tryptophan or the synthesis of quinone molecules. In an additional example, a viable, culturable, anaerobic gut bacterial strain that produces aryl hydrocarbon receptor agonists sufficient to stimulate host aryl hydrocarbon receptor pathways by microbial synthesis of flavonoids, phenazines, and/or quinones. Thus microbes that express or encode biosynthetic enzymes that participate in the synthesis of flavonoids, phenazines and/or quinones are identified as microbes that produce host aryl hydrocarbon receptor agonists. In one embodiment, the biosynthetic enzymes include the last enzyme in the pathway that catalyzes the final biosynthetic reaction producing e.g., flavonoids, phenazine or quinone compounds.

In some embodiments, a microbial consortium comprises at least one bacterial species that produces compounds capable of stimulating the pregnane X receptor that e.g., has beneficial effects on gut barrier function and/or the development of regulatory T cell processes. Non-limiting examples of compounds that stimulate the pregnane X receptor include (i) desmolase, (ii) compounds or intermediates of hydroxysteroid dehydrogenase activity, or (iii) compounds or intermediates derived from flavonoid metabolism enzymes. Thus, bacteria that encode and express steroid desmolase and/or hydroxysteroid dehydrogenase enzymes are expected to produce compounds that stimulate the pregnane X receptor. Clostridium sardiniensis and Clostridium scindens are non-limiting examples of bacterial species that produce compounds capable of stimulating the pregnane X receptor.

In some embodiments, the microbial consortium comprises at least one bacterial species that produces compounds capable of stimulating the RAR-related orphan receptor gamma (RORgamma) pathways, for example, to stimulate development of regulatory T cell responses via direct stimulation of RORgamma-activated pathways in gut antigen presenting cells and/or epithelial cells that then stimulate regulatory T cell responses. In one embodiment, the viable, culturable, anaerobic gut bacterial strain that produces compounds endogenously or by metabolizing ingested precursors, that is capable of stimulating the RORgamma (RAR-related orphan receptor gamma) pathways to stimulate development of regulatory T cell responses is a strain that expresses at least one cholesterol reductase and other enzymes capable of metabolizing sterol compounds. Non-limiting examples of microbes that produce compounds that stimulate the RORgamma pathway include Clostridium scindens, Clostridia hiranonsis, and Clostridium sardiniensis. In one instance, those species express bile acid transforming enzymes that can also produce RORgamma pathway agonists.

In some embodiments, a microbial consortium described herein improves gut function, for example, by stimulating host mucins and complex glycoconjugates and improving colonization by protective commensal species. In one embodiment, the microbial consortium comprises at least one bacterial species, such as Bacteroides vulgatus, that stimulates production of mucins and complex glycoconjugates by the host.

Immunomodulation: Other exemplary compositions useful for treatment of food allergy contain bacterial species capable of altering the proportion of immune subpopulations, e.g., T cell subpopulations, e.g., T_{regs} in the subject.

For example, immunomodulatory bacteria can increase or decrease the proportion of T_{reg} cells, Tₕ17 cells, Tₕ1 cells, or Tₕ2 cells in a subject. The increase or decrease in the proportion of immune cell subpopulations can be systemic, or it can be localized to a site of action of the colonized consortium, e.g., in the gastrointestinal tract or at the site of a distal dysbiosis. In some embodiments, a microbial consortium comprising immunomodulatory bacteria is used for treatment of food allergy based on the desired effect of the probiotic composition on the differentiation and/or expansion of subpopulations of immune cells in the subject.

In one embodiment, the microbial consortium contains immunomodulatory bacteria that increase the proportion of T_{reg} cells in a subject or in a particular location in a subject, e.g., the gut tissues. In one embodiment, a microbial consortium contains immunomodulatory bacteria that increase the proportion of Tₕ17 cells in a subject. In another embodiment, a microbial consortium contains immunomodulatory bacteria that decrease the proportion of Tₕ17 cells in a subject. In one embodiment, a microbial consortium contains immunomodulatory bacteria that increase the proportion of Tₕ1 cells in a subject. In another embodiment, a microbial consortium contains immunomodulatory bacteria that decrease the proportion of Tₕ1 cells in a subject. In one embodiment, a microbial consortium contains immunomodulatory bacteria that increase the proportion of Tₕ2 cells in a subject. In another embodiment, a microbial consortium contains immunomodulatory bacteria that decrease the proportion of Tₕ2 cells in a subject.

In one embodiment, a microbial consortium contains immunomodulatory bacteria capable of modulating the proportion of one or more of T_{reg} cells, Tₕ17 cells, Tₕ1 cells, Tₕ2 cells, and combinations thereof in a subject. Certain immune cell profiles can be particularly desirable to treat or prevent inflammatory disorders, such as food allergies. For example, in some embodiments, treatment or prevention of e.g., food allergy can be promoted by increasing numbers of T_{reg} cells and Tₕ2 cells, and decreasing numbers of Tₕ17 cells and Tₕ1 cells. Accordingly, a microbial consortium for the treatment or prevention of food allergy can contain a microbial consortium capable of promoting T_{reg} cells and Tₕ2 cells, and reducing Tₕ17 and Tₕ1 cells.

In one embodiment, the anaerobic gut bacterial strain in the methods and compositions described herein express agonists capable of binding to and modulating responses mediated by Toll-like receptors (TLR), CD14 and/or lipid binding proteins in antigen presenting cells, gut epithelial cells and/or T cells to promote the development of regulatory T cells. Non-limiting examples of TLR agonists include lipopolysaccharide (LPS), exopolysaccharides (PSA), peptidoglycan or CpG motifs produced by commensal members of Bacteroides, or lipoteichoic acids (LTA) produced by members of Clostridium. In one embodiment, an anaerobic gut bacterial strain that acts as a TLR agonist is selected from the following Table.

| Family | Genus | Species |
|---|---|---|
| Clostridieaceae | Clostridium, Hungatella | Hungatella hathawayi |
| Eubacteriaceae | Eubacterium | Eubacterium rectale |
| Erysipelotrichaceae | Erysipelatoclostridium | Erysipelatoclostridium |
| | (formerly species in genus | ramosum (Clostridium |
| | Clostridium) | ramosum) |
| Lachnospiraceae | Blautia, Butyrovibrio, Cellulosyliticum, | Butyrovibrio crossatus, |
| | Clostridium cluster XIVa species, | Roseburia intestinalis, |
| | Coprococcus, Dorea, Lachnospira, | Clostridium scindens, |
| | Robinsonella, Roseburia, | Clostridium hylemonae, |
| | | Clostridium symbiosum |
| Ruminococcaceae | Faecalibacterium,Ruminococcus, | Faecalibacterium pransnitzii, |
| | Subdoligranulum, Clostridium cluster | Subdoligranulum variabile |
| | XIVa species | |
| Bacteroidaceae | Bacteroides | Bacteroides thetaiotaomicron, |
| | | Bacteroides fragilis, |
| | | Bacteroides ovatus |
| Prophyromonadaceae | Parabacteroides, | Pambacteroides goldsteinii, |
| | Porphyromonas, Tannerella | Parabacteroides merdae, |
| | | Parabacteroides distasonis |
| Prevotellaceae | Prevotella | Prevotella tannerae |

Bile Acid Transformation: Primary bile acids (e.g., cholic and chenodeoxycholic acids in humans) are generated in the liver of mammals, including humans, mainly by conjugation with the amino acids taurine or glycine, and are secreted in bile. In the intestinal tract, primary bile acids are metabolized by microbes that transform the primary bile acids to secondary bile acids. Intestinal microbial transformation of primary bile acids can include deconjugation, deglucuronidation, oxidation of hydroxyl groups, reduction of oxo groups to yield epimeric hydroxyl bile acids, esterification, and dehydroxylation. Non-limiting examples of bacteria that perform deconjugation of primary bile acids include Bacteroides, Bifidobacterium, Clostridium, and Lactobacillus. Non-limiting examples of bacteria that perform oxidation and epimerization of primary bile acids include Bacteroides, Clostridium, Eggerthella, Eubacterium, Peptostreptococcus, and Ruminococcus Non-limiting examples of bacteria that perform 7-dehydroxylation of primary bile acids include Clostridium, and Eubacterium. Non-limiting examples of bacteria that perform esterification of primary bile acids include Bacteroides, Eubacterium, and Lactobacillus.

In one embodiment, a microbial consortium as described herein comprises at least one bacterial constituent that transforms bile acids by deconjugation. In another embodiment, a microbial consortium as described herein comprises at least one bacterial constituent that transforms bile acids by 7-dehydroxylation. In another embodiment, a microbial consortium as described herein comprises at least one bacterial constituent that transforms bile acids by esterification.

In one embodiment, a microbial consortium as described herein comprises at least 2, at least 3, at least 4, at least 5, at least 6, at least 7, at least 8, at least 9, at least 10, at least 11 or more bacterial constituents that perform bile acid transformation.

In one embodiment, a microbial consortium as described herein comprises 11 or fewer, 10 or fewer, 9 or fewer, 8 or fewer, 7 or fewer, 6 or fewer, 5 or fewer, 4 or fewer, 3 or fewer, 2 or fewer 1 or fewer or zero bacterial constituents that perform bile acid transformation, such as deconjugation, esterification or 7-dehydroxylation.

In one embodiment, a microbial consortium comprises at least one anaerobic gut bacterial strain that alone, or in combination, performs the full complement of bile acid transformations.

Targets of GP-Ha consortium members: The microbial consortium as described herein has multiple targets within the host subject. Representative targets are summarized in the following Table.

Targets of GP-IIa consortium members

| Host Target | B.fragilis | B. ovatus | B. vulgatus | P. distasonis | P. melaninogenica |
|---|---|---|---|---|---|
| Bile salt | + | + | + | + | + |
| transformation | | | | | |
| Mucin layer and | + | + | + | Unknown | Unknown |
| glycoconjugate | | | | | |
| modulation | | | | | |
| Immunostimulatory | + | + | + | + | + |
| T_{reg} induction | + | + | + | + | Unknown |

Engineered microbes: In some embodiments, one or more members of the microbial consortium comprises an engineered microbe(s). For example, engineered microbes include microbes harboring i) one or more introduced genetic changes, such change being an insertion, deletion, translocation, or substitution, or any combination thereof, of one or more nucleotides contained on the bacterial chromosome or on an endogenous plasmid, wherein the genetic change can result in the alteration, disruption, removal, or addition of one or more protein coding genes, non-protein-coding genes, gene regulatory regions, or any combination thereof, and wherein such change can be a fusion of two or more separate genomic regions or can be synthetically derived; ii) one or more foreign plasmids containing a mutant copy of an endogenous gene, such mutation being an insertion, deletion, or substitution, or any combination thereof, of one or more nucleotides; and iii) one or more foreign plasmids containing a mutant or non-mutant exogenous gene or a fusion of two or more endogenous, exogenous, or mixed genes. The engineered microbe(s) can be produced using techniques including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, or any combination thereof.

Excluded Bacteria: In one embodiment, a microbial consortium does not include an organism conventionally classified as a pathogenic or opportunistic organism. It is possible that a function shared by all members of a given taxonomic group could be beneficial, e.g., for providing particular metabolites, yet for other reasons the overall effect of one or more particular members of the group is not beneficial and is, for example, pathogenic. Clearly, members of a given taxonomic group that cause pathogenesis, e.g., acute gastrointestinal pathologies, are to be excluded from the therapeutic or preventive methods and compositions described herein.

In one embodiment, the bacterial composition does not comprise at least one of: Acidaminococcus intestinalis, Escherichia coli, Lactobacillus casei, Lactobacillus paracasei, Raoultella sp., and Streptococcus mitis.

In another embodiment, the bacterial composition does not comprise at least one of Barnesiella intestinihominis; Lactobacillus reuteri; Enterococcus hirae, Enterococcus faecium, or Enterococcus durans; Anaerostipes caccae or Clostridium indolis; Staphylococcus wameri or Staphylococcus pasteuri; and Adlercreutzia equolifaciens.

In another embodiment, the bacterial composition does not comprise at least one of Clostridium botulinum, Clostridium cadaveris, Clostridium chauvoei, Clostridium clostridioforme, Clostridium cochlearium, Clostridium difficile, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputricum, Clostridium perfringens, Clostridium piliforme, Clostridium putrefaciens, Clostridium putrificum, Clostridium septicum, Clostridium sordellii, Clostridium sphenoides, and Clostridium tetani.

In another embodiment, the bacterial composition does not comprise at least one of Escherichia coli, and Lactobacillus johnsonii.

In another embodiment, the bacterial composition does not comprise at least one of Clostridium innocuum, Clostridium butyricum, Escherichia coli, and Blautia producta (previously known as Peptostreptococcus productus).

In another embodiment, the bacterial composition does not comprise at least one of Eubacteria, Fusobacteria, Propionibacteria, Escherichia coli, and Gemmiger.

In another embodiment, the compositions described herein do not comprise pathogenic bacteria such as e.g., Yersinia, Vibrio, Treponema, Streptococcus, Staphylococcus, Shigella, Salmonella, Rickettsia, Orientia, Pseudomonas, Neisseria, Mycoplasma, Mycobacterium, Listeria, Leptospira, Legionella, Klebsiella, Helicobacter. Haemophilus, Francisella, Escherichia, Ehrlichia, Enterococcus, Coxiella, Corynebacterium, Chlamydia, Chlamydophila, Campylobacter, Burkholderia, Brucella, Borrelia, Bordetella, Bacillus, multi-drug resistant bacteria, extended spectrum beta-lactam resistant Enterococci (ESBL), Carbapenem-resistant Enterobacteriaceae (CRE), and vancomycin-resistant Enterococci (VRE).

In other embodiments, the compositions described herein do not comprise pathogenic species or species, such as Aeromonas hydrophila, Campylobacter fetus, Plesiomonas shigellodes, Bacillus cereus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enterotoxigenic Escherichia coli (such as, but not limited to, LT and/or ST), Escherichia coli 0157:H7, Helicobacter pylori, Klebsiella pneumonia, Listeria monocytogenes, Plesiomonas shigelloides, Salmonella spp, Salmonella typhi, Salmonella paratyphi, Shigella spp., Staphylococcus spp., Staphylococcus aureus, vancomycin-resistant Enterococcus spp., Vibrio spp., Vibrio cholerae, Vibrio parahaemolyticus, Vibrio vulnificus, and Yersinia enterocolitica.

In one embodiment, the microbial consortia and compositions thereof do not comprise Escherichia coli, Klebsiella pneumoniae. Proteus mirabilis. Enterobacter cloacae, and/or Bilophila wadsworthia.

Reduction of pathogenic organisms: In some embodiments, compositions comprising a microbial consortium as described herein offer a protective or therapeutic effect against dysbiosis or against infection by one or more GI pathogens of interest. In one embodiment, a microbial consortium as described herein reduces the biomass of one or more dysbiotic or pathogenic bacterial species or strains.

In one embodiment, a microbial consortium as described herein decreases the biomass of one or more dysbiotic or pathogenic bacterial species or strains by at least 10% compared to the biomass of the one or more dysbiotic or pathogenic bacterial species or strains in the absence of treatment with such microbial consortium. In other embodiments the biomass of one or more pathogenic bacterial species or strains is decreased by at least 20%, at least 30%, at least 40%, at least 50%, at least 60%, at least 70%, at least 80%, at least 90%, at least 95%, at least 99%, or even 100% (i.e., below detectable limits of the assay) as compared to the biomass of the dysbiotic or pathogenic bacterial species or strains in the gut of the subject prior to treatment with the microbial consortium or compositions thereof.

In some embodiments, a microbial consortium as described herein alters the gut environment such that the number, biomass, or activity of one or more dysbiotic or pathogenic organisms is decreased by at least 10% (e.g., at least 20%, at least 30%, at least 40%, at least 50%, at least 60%, at least 70%, at least 80%, at least 90%, at least 95%, at least 99%, or even 100% (i.e., below detectable limits of the assay)). As but one example, colonization of Bacteroides reduces the biomass of dysbiotic species in the Enterobacteriaceae or Desulfonovibriacaea Families.

In some embodiments, the pathogenic bacterium is selected from the group consisting of Yersinia, Vibrio, Treponema, Streptococcus. Staphylococcus, Escherichia/Shigella. Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Clostridium, Chlamydia, Chlamydophila, Campylobacter, Burkholderia, Brucella, Borrelia, Bordetella, Bifidobacterium, Bacillus, Bilophila, Desulfovibrio, multi-drug resistant bacteria, extended spectrum beta-lactam resistant Enterococci (ESBL), Carbapenem-resistant Enterobacteriaceae (CRE), and vancomycin-resistant Enterococci (VRE).

In some embodiments, these pathogens include, but are not limited to, Aeromonas hydrophila, Campylobacter fetus. Plesiomonas shigelloides, Bacillus cereus. Campylobacter jejuni, Clostridium botulinum, Clostridium difficile, Clostridium perfringens, enteroaggregative Escherichia coli, entero hemorrhagic Escherichia coli, enteroinvasive Escherichia coli, enterotoxigenic Escherichia coli (such as, but not limited to, LT and/or ST), Escherichia coli 0157:1H7, Helicobacter pylori, Klebsiella pneumonia, Listeria monocytogenes, Plesiomonas shigelloides, Salmonella spp., Salmonella typhi, Salmonella paratyphi, Shigella spp., Staphylococcus spp., Staphylococcus aureus, vancomycin-resistant Enterococcus spp., Vibrio spp., Vibrio cholerae, Vibrio parahaemolyticus, Vibrio vulnificus, and Yersinia enterocolitica.

In one embodiment, the pathogen of interest is at least one pathogen chosen from Clostridium difficile, Salmonella spp., pathogenic Escherichia coli, vancomycin-resistant Enterococcus spp., and extended spectrum beta-lactam resistant Enterococci (ESBL).

Methods for testing the efficacy of the compositions comprising a microbial composition to reduce the number, biomass, or activity of one or more dysbiotic or pathogenic organisms are discussed in the following. While certain of the methods are described in the following in terms of assaying reduced number, biomass or activity of Clostridium difficile, one of skill in the art can readily adapt the methods to measure the number, biomass or activity of one or more further microbial species or strains.

In one embodiment, provided is an in Vitro Assay utilizing competition between the bacterial compositions or subsets thereof and Clostridium difficile or other dysbiotic or pathogenic strain. This test in known in the art and as such is not described in detail herein.

In another embodiment, provided is an In Vitro Assay utilizing 10% (wt/vol) Sterile-Filtered Feces. This assay tests for the protective effect of the bacterial compositions and screens in vitro for combinations of microbes that inhibit the growth of a given pathogenic or dysbiotic microbe. The assay can operate in automated high-throughput or manual modes. Under either system, human or animal feces can be re-suspended in an anaerobic buffer solution, such as pre-reduced PBS or other suitable buffer, the particulate removed by centrifugation, and filter sterilized. This 10% sterile-filtered feces material serves as the base media for the in vitro assay. To test a bacterial composition, an investigator can add it to the sterile-filtered feces material for a first incubation period and then can inoculate the incubated microbial solution with a pathogenic or dysbiotic microbe of interest for a second incubation period. The resulting titer of the pathogenic or dysbiotic microbe is quantified by any number of methods such as those described below, and the change in the amount of pathogen is compared to standard controls including the pathogenic or dysbiotic microbe cultivated in the absence of the bacterial composition. The assay is conducted using at least one control. Feces from a healthy subject can be used as a positive control. As a negative control, antibiotic-treated feces or heat-treated feces can be used. Various bacterial compositions can be tested in this material and the bacterial compositions optionally compared to the positive and/or negative controls. The ability to inhibit the growth of a pathogenic or dysbiotic microbe can be measured by plating the incubated material on selective media and counting colonies. After competition between the bacterial composition and the pathogenic or dysbiotic microbe, each well of the m vitro assay plate is serially diluted ten-fold six times, and plated on selective media. For Clostridium difficile this would include, for example, cycloserine cefoxitin mannitol agar (CCMA) or cycloserine cefoxitin fructose agar (CCFA), and incubated. Colonies of the pathogenic or dysbiotic microbes are then counted to calculate the concentration of viable cells in each well at the end of the competition.

Alternatively, the ability to inhibit the growth of a pathogenic or dysbiotic species can be measured by quantitative PCR (qPCR). Standard techniques can be followed to generate a standard curve for the pathogenic or dysbiotic strain of interest. Genomic DNA can be extracted from samples using commercially-available kits, such as the Mo Bio Powersoil®-htp 96 Well Soil DNA Isolation Kit (Mo Bio Laboratories, Carlsbad, Calif.), the Mo Bio Powersoil® DNA Isolation Kit (Mo Bio Laboratories, Carlsbad, Calif.), or the QIAamp DNA Stool Mini Kit (QIAGEN, Valencia, Calif.) according to the manufacturer's instructions. The qPCR can be conducted using HotMasterMix (SPRIME, Gaithersburg, Md.) and primers specific for the pathogenic or dysbiotic microbe of interest, and can be conducted on a MicroAmp® Fast Optical 96-well Reaction Plate with Barcode (0.1 mL) (Life Technologies, Grand Island, N.Y.) and performed on a BioRad C1000™ Thermal Cycler equipped with a CFX96™ Real-Time System (BioRad, Hercules, Calif.), with fluorescent readings of the FAM and ROX channels. The Cq value for each well on the FAM channel is determined by the CFX Manager™ software version 2.1. The log₁₀ (cfu/ml) of each experimental sample is calculated by inputting a given sample's Cq value into linear regression model generated from the standard curve comparing the Cq values of the standard curve wells to the known log₁₀ (cfu/ml) of those samples. The skilled artisan can employ alternative qPCR modes.

Also provided are In Vivo Assays establishing the protective effect of bacterial compositions. The assay is described in terms of protective effect against Clostridium difficile, but can be adapted by one of skill in the art for other pathogens or dysbiotic species. Provided is an in vivo mouse model to test for the protective effect of the bacterial compositions against Clostridium difficile. In this model (based on Chen, et al., Gastroenterology 135(6) 1984-1992 (2008)), mice are made susceptible to Clostridium difficile by a 7 day treatment (days −12 to −5 of experiment) with 5 to 7 antibiotics (including kanamycin, colistin, gentamycin, metronidazole and vancomycin and optionally including ampicillin and ciprofloxacin) delivered via their drinking water, followed by a single dose with Clindamycin on day −3, then challenged three days later on day 0 with 104 spores of Clostridium difficile via oral gavage (i.e., cro-gastric lavage). Bacterial compositions can be given either before (prophylactic treatment) or after (therapeutic treatment) Clostridium difficile gavage. Further, bacterial compositions can be given after (optional) vancomycin treatment to assess their ability to prevent recurrence and thus suppress the pathogen in vivo. The outcomes assessed each day from day −1 to day 6 (or beyond, for prevention of recurrence) are weight, clinical signs, mortality and shedding of Clostridium difficile in the feces. Weight loss, clinical signs of disease and Clostridium difficile shedding are typically observed without treatment. Vancomycin provided by oral gavage on days −1 to 4 protects against these outcomes and serves as a positive control. Clinical signs are subjective, and scored each day by the same experienced observer. Animals that lose greater than or equal to 25% of their body weight are euthanized and counted as infection-related mortalities. Feces are gathered from mouse cages (5 mice per cage) each day, and the shedding of Clostridium difficile spores is detected in the feces using a selective plating assay as described for the in vitro assay above, or via qPCR for the toxin gene. The effects of test materials including 10% suspension of human feces (as a positive control), bacterial compositions, or PBS (as a negative vehicle control), are determined by introducing the test article in a 0.2 mL volume into the mice via oral gavage on day −1, one day prior to Clostridium difficile challenge, on day 1, 2 and 3 as treatment or post-vancomycin treatment on days 5, 6, 7 and 8. Vancomycin, as discussed above, is given on days 1 to 4 as another positive control. Alternative dosing schedules and routes of administration (e.g. rectal) may be employed, including multiple doses of test article, and 10³ to 10¹³ of a given organism or composition may be delivered.

Enhancement of beneficial organisms: In some embodiments, compositions comprising a microbial consortium offer a therapeutic effect of enhancing beneficial organisms in the GI tract. In one embodiment, a microbial consortium as described herein increases the biomass of one or more beneficial bacterial species by at least 10%. In other embodiments the biomass of one or more beneficial bacterial species is increased by at least 20%, at least 30%, at least 40%, at least 50%, at least 60%, at least 70%, at least 80%, at least 90%, at least 1-fold, at least 2-fold, at least 5-fold, at least 10-fold, at least 50-fold, at least 100-fold, at least 500-fold, at least 1000-fold, at least 5000-fold, at least 10,000-fold, at least 15,000-fold or at least 20,000-fold over the biomass of the beneficial bacterial species in the gut of the subject prior to treatment with the microbial consortium or compositions thereof. In one embodiment, the beneficial organisms are commensal bacterial species that currently reside or exist in the gut. In another embodiment, the beneficial organisms are one or more of the bacterial species in the microbial consortium itself.

In some embodiments, a microbial consortium as described herein alters the gut environment such that the number, biomass, or activity of one or more beneficial organisms is increased by at least 10% (e.g., by at least 20%, at least 30%, at least 40%, at least 50%, at least 60%, at least 70%, at least 80%, at least 90%, at least 1-fold, at least 2-fold, at least 5-fold, at least 10-fold, at least 50-fold, at least 100-fold, at least 500-fold, at least 1000-fold, at least 5000-fold, at least 10,000-fold, at least 15,000-fold or at least 20,000-fold). For example, the microbial consortium stimulates the host's production of mucins and complex glycoconjugates to improve gut barrier function and colonization of beneficial organisms, additional probiotic compositions, or the microbial consortium itself. In some embodiments, the microbial composition for enhancing the biomass and/or activity of beneficial organisms comprises e.g., Bacteroides species, which enhances colonization by other Bacteroidetes and Clostridiales. In some embodiments, the microbial consortium influences gut pH, reduction of oxygen tension, secretion of glycosidases, and improving the reduction potential of the gut lumen to enhance the colonization of beneficial organisms.

In another embodiment, the beneficial species comprises a Clostridium spp, such as Clostridium ramosum, Clostridium scindens, Clostridium hiranonsis, Clostridium bifermentans, Clostridium leptum, Clostridium sardiniensis, Clostridium hathewayi, Clostridium nexile, Clostridium hylemonae, Clostridium glycyrrhizinilyticum, Clostridium lavalense, Clostridium fimetarium, Clostridium symbiosum, or Clostridium sporosphaeroides.

### Characterization of Bacteria and Bacterial Consortia

In certain embodiments, methods are provided for testing certain characteristics of compositions comprising a microbial consortium. For example, the sensitivity of bacterial compositions to certain environmental variables is determined, e.g., in order to select for particular desirable characteristics in a given composition, formulation and/or use. For example, the bacterial constituents of the composition can be tested for pH resistance, bile acid resistance, and/or antibiotic sensitivity, either individually on a constituent-by-constituent basis or collectively as a bacterial composition comprised of multiple bacterial constituents (collectively referred to in this section as a microbial consortium).

pH Sensitivity Testing: If a pharmaceutical composition will be administered other than to the colon or rectum (i.e., for example, an oral route), optionally testing for pH resistance enhances the selection of microbes or therapeutic compositions that will survive at the highest yield possible through the varying pH environments of the distinct regions of the GI tract or genitourinary tracts. Understanding how the bacterial compositions react to the pH of the GI or genitourinary tracts also assists in formulation, so that the number of microbes in a dosage form can be increased if beneficial and/or so that the composition can be administered in an enteric coated capsule or tablet or with a buffering or protective composition.

As the pH of the stomach can drop to a pH of 1 to 2 after a high-protein meal for a short time before physiological mechanisms adjust it to a pH of 3 to 4 and often resides at a resting pH of 4 to 5, and as the pH of the small intestine can range from a pH of 6 to 7.4, bacterial compositions can be prepared that survive these varying pH ranges (specifically wherein at least 1%, 5%, 10%, 15%, 20%, 25%, 30%, 40%, 50%, 60%, 70%, 80%, 90%, or as much as 100% of the bacteria can survive gut transit times through various pH ranges). This can be tested by exposing the bacterial composition to varying pH ranges for the expected gut transit times through those pH ranges. Therefore, as a non-limiting example only, 18-hour cultures of compositions comprising one or more bacterial species or species can be grown in standard media, such as gut microbiota medium (“GMM”, see Goodman et al, PNAS 108(15):6252-6257 (2011)) or another animal-products-free medium, with the addition of pH adjusting agents for a pH of 1 to 2 for 30 minutes, a pH of 3 to 4 for 1 hour, a pH of 4 to 5 for 1 to 2 hours, and a pH of 6 to 7.4 for 2.5 to 3 hours. An alternative method for testing stability to acid is described in e.g., U.S. Pat. No. 4,839,281. Survival of bacteria can be determined by culturing the bacteria and counting colonies on appropriate selective or non-selective media.

Bile Acid Sensitivity Testing: Additionally, in some embodiments, testing for bile-acid resistance enhances the selection of microbes or therapeutic compositions that will survive exposures to bile acid during transit through the GI tract. Bile acids are secreted into the small intestine and can, like pH, affect the survival of bacterial compositions. This can be tested by exposing the compositions to bile acids for the expected gut exposure time to bile acids. For example, bile acid solutions can be prepared at desired concentrations using 0.05 mM Tris at pH 9 as the solvent. After the bile acid is dissolved, the pH of the solution can be adjusted to 7.2 with 10% HCl. Bacterial components of the therapeutic compositions can be cultured in 2.2 ml of a bile acid composition mimicking the concentration and type of bile acids in the patient, TO ml of 10% sterile-filtered feces media and 0.1 ml of an 18-hour culture of the given strain of bacteria. Incubations can be conducted for from 2.5 to 3 hours or longer. An alternative method for testing stability to bile acid is described in e.g., U.S. Pat. No. 4,839,281. Survival of bacteria can be determined by culturing the bacteria and counting colonies on appropriate selective or non-selective media.

Antibiotic Sensitivity Testing: As a further optional sensitivity test, the bacterial components of the microbial compositions can be tested for sensitivity to antibiotics. In one embodiment, the bacterial components can be chosen so that they are sensitive to antibiotics such that if necessary they can be eliminated or substantially reduced from the patient's gastrointestinal tract by at least one antibiotic targeting the bacterial composition.

Adherence to Gastrointestinal Cells. The compositions can optionally be tested for the ability to adhere to gastrointestinal cells. A method for testing adherence to gastrointestinal cells is described in e.g., U.S. Pat. No. 4,839,281.

Identification of Immunomodulatory Bacteria. In some embodiments, immunomodulatory bacteria are identified by the presence of nucleic acid sequences that modulate sporulation. In particular, signature sporulation genes are highly conserved across members of distantly related genera including Clostridium and Bacillus. Traditional approaches of forward genetics have identified many, if not all, genes that are essential for sporulation (spo). The developmental program of sporulation is governed in part by the successive action of four compartment-specific sigma factors (appearing in the order σF, σE, σG and σK), whose activities are confined to the forespore (σF and σG) or the mother cell (σE and σK). In other embodiments, immunomodulatory bacteria are identified by the biochemical activity of DPA producing enzymes or by analyzing DPA content of cultures. As part of the bacterial sporulation, large amounts of DPA are produced, and comprise 5-15% of the mass of a spore. Because not all viable spores germinate and grow under known media conditions, it is difficult to assess a total spore count in a population of bacteria. As such, a measurement of DPA content highly correlates with spore content and is an appropriate measure for characterizing total spore content in a bacterial population.

In other embodiments, immunomodulatory bacteria are identified by screening bacteria to determine whether the bacteria induce secretion of pro-inflammatory or anti-inflammatory cytokines by host cells. For example, human or mammalian cells capable of cytokine secretion, such as immune cells (e.g., PBMCs, macrophages, T cells, etc.) can be exposed to candidate immunomodulatory bacteria, or supernatants obtained from cultures of candidate immunomodulatory bacteria, and changes in cytokine expression or secretion can be measured using standard techniques, such as ELISA, immunoblot, Luminex™, antibody array, quantitative PCR, microarray, etc. Bacteria can be selected for inclusion in a microbial consortium based on the ability to induce a desired cytokine profile in human or mammalian cells. For example, anti-inflammatory bacteria can be selected for inclusion (or alternatively exclusion) in a microbial consortium or composition thereof, based on the ability to induce secretion of one or more anti-inflammatory cytokines, and/or the ability to reduce secretion of one or more pro-inflammatory cytokines. Anti-inflammatory cytokines include, for example, IL-10, IL-13, IL-9, IL-4, IL-5, and combinations thereof. Other inflammatory cytokines include, for example, TGFβ. Pro-inflammatory cytokines include, for example, IFNγ, IL-12p70, IL-1α, IL-6, IL-8, MCP1, MEP1α, MIP1β, TNFα, and combinations thereof. In some embodiments, anti-inflammatory bacteria can be selected for inclusion in a microbial consortium based on the ability to modulate secretion of one or more anti-inflammatory cytokines and/or the ability to reduce secretion of one or more pro-inflammatory cytokines by a host cell induced by a bacterium of a different type (e.g., a bacterium from a different species or from a different strain of the same species).

In other embodiments, immunomodulatory bacteria are identified by screening bacteria to determine whether the bacteria impact the differentiation and/or expansion of particular subpopulations of immune cells. For example, candidate bacteria can be screened for the ability to promote differentiation and/or expansion of T_{reg} cells, Tₕ17 cells, Tₕ1 cells and/or Tₕ2 cells from precursor cells, e.g., naive T cells. By way of example, naïve T cells can be cultured in the presence of candidate bacteria or supernatants obtained from cultures of candidate bacteria, and numbers of T_{reg} cells, Th17 cells, Tₕ1 cells and/or Tₕ2 cells can be determined using standard techniques, such as FACS analysis. Markers indicative of T_{reg} cells include, for example, CD25⁺CD127^{lo}. Markers indicative of Tₕ17 cells include, for example, CXCR3⁻CCR6⁺. Markers indicative of Tₕ1 cells include, for example, CD4⁺, CXCR3⁺, and CCR6⁻. Markers indicative of Tₕ2 cells include, for example, CD4⁺, CCR4⁺, and CXCR3⁻, CCR6⁻. Other markers indicative of particular T cells sub populations are known in the art, and may be used in the assays described herein, e.g., to identify populations of immune cells impacted by candidate immunomodulatory bacteria. Bacteria can be selected for inclusion (or exclusion) in a microbial consortium based on the ability to promote differentiation and/or expansion of a desired immune cell subpopulation.

In other embodiments, immunomodulatory bacteria are identified by screening bacteria to determine whether the bacteria secrete short chain fatty acids (SCFA), such as, for example, butyrate, acetate, propionate, or valerate, or combinations thereof. For example, secretion of short chain fatty acids into bacterial supernatants can be measured using standard techniques. In one embodiment, bacterial supernatants can be screened to measure the level of one or more short chain fatty acids using NMR, mass spectrometry (e.g., GC-MS, tandem mass spectrometry, matrix-assisted laser desorption/ionization, etc.), ELISA, or immunoblot. Expression of bacterial genes responsible for production of short chain fatty acids can also be determined by standard techniques, such as Northern blot, microarray, or quantitative PCR.

Exemplary minimal microbial consortia. Minimal microbial consortia are shown herein in the Examples section and can prevent and/or treat existing symptoms of a food allergy. These exemplary minimal microbial consortia should not be construed as limiting and are intended only for the better understanding of the methods and compositions described herein.

In one embodiment, a minimal microbial consortium consists essentially of: Clostridium ramosum, Clostridium scindens, Clostridium hiranonsis, Clostridium bifermentans, Clostridium lepturn and Clostridium sardiniensis.

In one embodiment, a minimal microbial consortium consisting essentially of: Clostridium ramosum, Clostridium scindens, Clostridium hiranonsis, Clostridium bifermentans, Clostridium lepturn and Clostridium sardiniensis is used in the prevention and/or treatment of existing allergic reactions to food.

In one embodiment, a minimal microbial consortium consists essentially of: Bacteroides fragilis, Bacteroides ovatus, Bacteroides vulgatus, Parabacteroides distasonis, and Prevotella melaninogenica.

In one embodiment, a minimal microbial consortium consisting essentially of: Bacteroides fragilis, Bacteroides ovatus, Bacteroides vulgatus, Parabacteroides distasonis, and Prevotella melaninogenica is used to treat an existing atopic disease or disorder, allergic reaction, e.g., an allergy to food.

By “consists essentially of” in this context is meant that if the addition of another microbe does not improve the treatment or prevention of allergy as described and defined herein, that microbe is not essential to the protective or therapeutic effect.

### Prebiotics

A prebiotic is a selectively fermented ingredient that allows specific changes, both in the composition and/or activity in the gastrointestinal microbiota, that confers neutral or positive benefits upon host well-being and health. Prebiotics can include complex carbohydrates, amino acids, peptides, or other nutritional components useful for the survival, colonization and persistence of the bacterial composition. Prebiotics include, but are not limited to, amino acids, biotin, fructooligosaccharide, galactooligosaccharides, inulin, lactulose, mannan oligosaccharides, oligofructose-enriched inulin, oligofructose, oligodextrose, tagatose, trans-galactooligosaccharide, and xylooligosaccharides.

Suitable prebiotics are usually plant-derived complex carbohydrates, oligosaccharides or polysaccharides. Generally, prebiotics are indigestible or poorly digested by humans and serve as a food source for bacteria. Prebiotics, which can be used in the pharmaceutical dosage forms, and pharmaceutical compositions provided herein include, without limitation, galactooligosaccharides (GOS), trans-galactooligosaccharides, fructooligosaccharides or oligolfuctose (FOS), inulin, oligofructose-enriched inulin, lactulose, arabinoxylan, xylooligosaccharides (XOS), mannooligosaccharides, gum guar, gum Arabic, tagatose, amylose, amylopectin, xylan, pectin, and the like and combinations of thereof. Prebiotics can be found in certain foods, e.g., chicory root, Jerusalem artichoke, Dandelion greens, garlic, leek, onion, asparagus, wheat bran, wheat flour, banana, milk, yogurt, sorghum, burdock, broccoli, Brussels sprouts, cabbage, cauliflower, collard greens, kale, radish and rutabaga, and miso. Alternatively, prebiotics can be purified or chemically or enzymatically synthesized.

In some embodiments, the composition comprises at least one prebiotic. In one embodiment, the prebiotic is a carbohydrate. In some embodiments, the composition comprises a prebiotic mixture, which comprises at least one carbohydrate. A carbohydrate is a sugar or polymer of sugars. The terms “saccharide,” “polysaccharide,” “carbohydrate,” and “oligosaccharide” can be used interchangeably. Most carbohydrates are aldehydes or ketones with many hydroxyl groups, usually one on each carbon atom of the molecule. Carbohydrates generally have the molecular formula (CH₂O)n. A carbohydrate can be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates can contain modified saccharide units, such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2′-fluororibose, deoxyribose, and hexose). Carbohydrates can exist in many different forms, for example, conformers, cyclic forms, acyclic forms, stereoisomers, tautomers, anomers, and isomers. Carbohydrates can be purified from natural (e.g., plant or microbial) sources (i.e., they are enzymatically synthesized), or they can be chemically synthesized or modified.

Suitable prebiotic carbohydrates can include one or more of a carbohydrate, carbohydrate monomer, carbohydrate oligomer, or carbohydrate polymer. In certain embodiments, the pharmaceutical composition or dosage form comprises at least one type of microbe and at least one type of non-digestible saccharide, which includes non-digestible monosaccharides, non-digestible oligosaccharides, or non-digestible polysaccharides. In one embodiment, the sugar units of an oligosaccharide or polysaccharide can be linked in a single straight chain or can be a chain with one or more side branches. The length of the oligosaccharide or polysaccharide can vary from source to source. In one embodiment, small amounts of glucose can also be contained in the chain. In another embodiment, the prebiotic composition can be partially hydrolyzed or contain individual sugar moieties that are components of the primary oligosaccharide (see e.g., U.S. Pat. No. 8,486,668).

Prebiotic carbohydrates can include, but are not limited to monosaccharides (e.g., trioses, tetroses, pentoses, aldopentoses, ketopentoses, hexoses, cyclic hemiacetals, ketohexoses, heptoses) and multimers thereof, as well as epimers, cyclic isomers, stereoisomers, and anomers thereof. Non-limiting examples of monosaccharides include (in either the L- or D-conformation) glyceraldehyde, threose, ribose, altrose, glucose, mannose, talose, galactose, gulose, idose, lyxose, arabanose, xylose, allose, erythrose, erythrulose, tagalose, sorbose, ribulose, psicose, xylulose, fructose, dihydroxyacetone, and cyclic (alpha or beta) forms thereof. Multimers (disaccharides, trisaccharides, oligosaccharides, polysaccharides) thereof include, but are not limited to, sucrose, lactose, maltose, lactulose, trehalose, cellobiose, kojibiose, nigerose, isomaltose, sophorose, laminaribiose, gentioboise, turanose, maltulose, palatinose, gentiobiulose, mannobiose, melibiulose, rutinose, rutinulose, xylobiose, primeverose, amylose, amylopectin, starch (including resistant starch), chitin, cellulose, agar, agarose, xylan, glycogen, bacterial polysaccharides such as capsular polysaccharides, LPS, and peptidoglycan, and biofilm exopolysaccharide (e.g., alginate, EPS), N-linked glycans, and O-linked glycans. Prebiotic sugars can be modified and carbohydrate derivatives include amino sugars (e.g., sialic acid, N-acetylglucosamine, galactosamine), deoxy sugars (e.g., rhamnose, fucose, deoxyribose), sugar phosphates, glycosylamines, sugar alcohols, and acidic sugars (e.g., glucuronic acid, ascorbic acid).

In one embodiment, the prebiotic carbohydrate component of the pharmaceutical composition consists essentially of one or more non-digestible saccharides.

In one embodiment, the prebiotic carbohydrate component of the pharmaceutical composition allows the commensal colonic microbiota, comprising microorganisms associated with a healthy-state microbiome or presenting a low risk of a patient developing an autoimmune or inflammatory condition, to be regularly maintained. In one embodiment, the prebiotic carbohydrate allows the co-administered or co-formulated microbe or microbes to engraft, grow, and/or be regularly maintained in a mammalian subject.

In some embodiments, the mammalian subject is a human subject, for example, a human subject having or suspected of having a food allergy. In some embodiments, the prebiotic favors the growth of an administered microbe, wherein the growth of the administered microbe and/or the fermentation of the administered prebiotic by the administered microbe slows or reduces the growth of a pathogen or pathobiont. For example, FOS, neosugar, or inulin promotes the growth of acid-forming bacteria in the colon such as bacteria belonging to the genus Lactobacillus or Bifidobacterium and Lactobacillus acidophilus and Bifidobacterium bifidus can play a role in reducing the number of pathogenic bacteria in the colon (see e.g., U.S. Pat. No. 8,486,668). Other polymers, such as various galactans, lactulose, and carbohydrate based gums, such as psyllium, guar, carrageen, gellan, and konjac, are also known to improve gastrointestinal (GI) health.

In some embodiments, the prebiotic comprises one or more of GOS, lactulose, raffinose, stachyose, lactosucrose, FOS (i.e., oligofructose or oligofructan), inulin, isomalto-oligosaccharide, xylo-oligosaccharide, paratinose oligosaccharide, transgalactosylated oligosaccharides (i.e., transgalacto-oligosaccharides), transgalactosylate disaccharides, soybean oligosaccharides (i.e., soy oligosaccharides), gentiooligosaccharides, glucooligosaccharides, pecticoligosaccharides, palatinose polycondensates, difructose anhydride III, sorbitol, maltitol, lactitol, polyols, polydextrose, reduced paratinose, cellulose, β-glucose, β-galactose, β-fructose, verbascose, galactinol, and β-glucan, guar gum, pectin, high, sodium alginate, and lambda carrageenan, or mixtures thereof. The GOS may be a short-chain GOS, a long-chain GOS, or any combination thereof. The FOS can be a short-chain FOS, a long-chain FOS, or any combination thereof.

In some embodiments, the prebiotic composition comprises two carbohydrate species (non-limiting examples being a GOS and FOS) in a mixture of at least 1:1, at least 2:1, at least 5:1, at least 9:1, at least 10:1, about 20:1, or at least 20:1.

In some embodiments, the prebiotic comprises a mixture of one or more non-digestible oligosaccharides, non-digestible polysaccharides, free monosaccharides, non-digestible saccharides, starch, or non-starch polysaccharides.

Oligosaccharides are generally considered to have a reducing end and a non-reducing end, whether or not the saccharide at the reducing end is in fact a reducing sugar. Most oligosaccharides described herein are described with the name or abbreviation for the non-reducing saccharide (e.g., Gal or D-Gal), preceded or followed by the configuration of the glycosidic bond (a or (3), the ring bond, the ring position of the reducing saccharide involved in the bond, and then the name or abbreviation of the reducing saccharide (e.g., Glc or D-Glc). The linkage (e.g., glycosidic linkage, galactosidic linkage, glucosidic linkage) between two sugar units can be expressed, for example, as 1,4, 1->4, or (1-4).

Both FOS and GOS are non-digestible saccharides. β glycosidic linkages of saccharides, such as those found in, but not limited to, FOS and GOS, make these prebiotics mainly non-digestible and unabsorbable in the stomach and small intestine a-linked GOS (a-GOS) is also not hydrolyzed by human salivary amylase, but can be used by Bifidobacterium bifidum and Clostridium butyricum (Yamashita A. et al., 2004. J. Appl. Glycosci. 51:115-122) FOS and GOS can pass through the small intestine and into the large intestine (colon) mostly intact, except where commensal microbes and microbes administered as part of a pharmaceutical composition are able to metabolize the oligosaccharides.

GOS (also known as galacto-oligosaccharides, galactooligosaccharides, trans-oligosaccharide (TOS), trans-galacto-oligosaccharide (TGOS), and trans-galactooligosaccharide) are oligomers or polymers of galactose molecules ending mainly with a glucose or sometimes ending with a galactose molecule and have varying degree of polymerization (generally the DP is between 2-20) and type of linkages. In one embodiment, GOS comprises galactose and glucose molecules. In another embodiment, GOS comprises only galactose molecules. In a further embodiment, GOS are galactose-containing oligosaccharides of the form of [β-D-Gal-(1-6)]ₙ-β-D-Gal-(1-4)-D-Glc wherein n is 2-20. In another embodiment, GOS are galactose-containing oligosaccharides of the form Glc α1-4-[β Gal 1-6)]ₙ where n=2-20. In another embodiment, GOS are in the form of α-D-Glc (1-4)-[β-D-Gal-(1-6)-]ₙ where n=2-20. Gal is a galactopyranose unit and Glc (or Glu) is a glucopyranose unit.

In one embodiment, a prebiotic composition comprises a GOS-related compound. A GOS-related compound can have the following properties: a) a “lactose” moiety; e.g., GOS with a gal-glu moiety and any polymerization value or type of linkage; or b) be stimulatory to “lactose fermenting” microbes in the human GI tract; for example, raffinose (gal-fru-glu) is a “related” GOS compound that is stimulatory to both lactobacilli and bifidobacteria.

Linkages between the individual sugar units found in GOS and other oligosaccharides include β-(1-6), β-(1-4), β-(1-3) and β-(1-2) linkages. In one embodiment, the administered oligosaccharides (e.g., GOS) are branched saccharides. In another embodiment, the administered oligosaccharides (e.g., GOS) are linear saccharides.

Alpha-GOS (also called alpha-bond GOS or alpha-linked GOS) are oligosaccharides having an alpha-galactopyranosyl group. Alpha-GOS comprises at least one alpha glycosidic linkage between the saccharide units. Alpha-GOS are generally represented by α-(Gal)ₙ (n usually represents an integer of 2 to 10) or α-(Gal)ₙGlc (n usually represents an integer of 1 to 9). Examples include a mixture of α-galactosylglucose, α-galactobiose, α-galactotriose, α-galactotetraose, and higher oligosaccharides. Additional non-limiting examples include melibiose, manninootriose, raffinose, stachyose, and the like, which can be produced from beat, soybean oligosaccharide, and the like.

Commercially available and enzyme synthesized alpha-GOS products are also useful for the compositions described herein. Synthesis of alpha-GOS with an enzyme is conducted utilizing the dehydration condensation reaction of α-galactosidase with the use of galactose, galactose-containing substance, or glucose as a substrate. The galactose-containing substance includes hydrolysates of galactose-containing substances, for example, a mixture of galactose and glucose obtained by allowing beta-galactosidase to act on lactose, and the like. Glucose can be mixed separately with galactose and be used as a substrate with α-galactosidase (see e.g., WO 02/18614). Methods of preparing alpha-GOS have been described (see e.g., EP 514551 and EP2027863).

In one embodiment, a GOS composition comprises a mixture of saccharides that are alpha-GOS and saccharides that are produced by transgalactosylation using β-galactosidase. In another embodiment, GOS comprises alpha-GOS. In another embodiment, alpha-GOS comprises α-(Gal)₂ from 10% to 100% by weight. In one embodiment, GOS comprises only saccharides that are produced by transgalactosylation using p-galactosidase.

In one embodiment, the pharmaceutical composition comprises, in addition to one or more microbes, an oligosaccharide composition that is a mixture of oligosaccharides comprising 1-20% by weight of di-saccharides, 1-20% by weight tri-saccharides, 1-20% by weight tetra-saccharides, and 1-20% by weight penta-saccharides. In another embodiment, an oligosaccharide composition is a mixture of oligosaccharides consisting essentially of 1-20% by weight of di-saccharides, 1-20% by weight tri-saccharides, 1-20% by weight tetra-saccharides, and 1-20% by weight penta-saccharides.

In one embodiment, a prebiotic composition is a mixture of oligosaccharides comprising 1-20% by weight of saccharides with a degree of polymerization (DP) of 1-3, 1-20% by weight of saccharides with DP of 4-6, 1-20% by weight of saccharides with DP of 7-9, and 1-20% by weight of saccharides with DP of 10-12, 1-20% by weight of saccharides with DP of 13-15.

In another embodiment, a prebiotic composition comprises a mixture of oligosaccharides comprising 50-55% by weight of di-saccharides, 20-30% by weight tri-saccharides, 10-20% by weight tetra-saccharide, and 1-10% by weight penta-saccharides. In one embodiment, a GOS composition is a mixture of oligosaccharides comprising 52% by weight of di-saccharides, 26% by weight tri-saccharides, 14% by weight tetra-saccharide, and 5% by weight penta-saccharides. In another embodiment, a prebiotic composition comprises a mixture of oligosaccharides comprising 45-55% by weight tri-saccharides, 15-25% by weight tetra-saccharides, 1-10% by weight penta-saccharides.

In certain embodiments, the composition comprises a mixture of neutral and acid oligosaccharides as disclosed in e.g., WO 2005/039597 (N.V. Nutricia) and US Patent Application 20150004130 In one embodiment, the acid oligosaccharide has a degree of polymerization (DP) between 1 and 5000. In another embodiment, the DP is between 1 and 1000. In another embodiment, the DP is between 2 and 250. If a mixture of acid oligosaccharides with different degrees of polymerization is used, the average DP of the acid oligosaccharide mixture is preferably between 2 and 1000. The acid oligosaccharide can be a homogeneous or heterogeneous carbohydrate. The acid oligosaccharides can be prepared from pectin, pectate, alginate, chondroitine, hyaluronic acids, heparin, heparane, bacterial carbohydrates, sialoglycans, fucoidan, fucooligosaccharides or carrageenan, and are preferably prepared from pectin or alginate. The acid oligosaccharides can be prepared by the methods described in e.g., WO 01/60378, which is hereby incorporated by reference. The acid oligosaccharide is preferably prepared from high methoxylated pectin, which is characterized by a degree of methoxylation above 50%. As used herein, the degree of methoxylation (also referred to as DE or degree of esterification) refers to the extent to which free carboxylic acid groups contained in the polygalacturonic acid chain have been esterified (e.g. by methylation). In some embodiments, the acid oligosaccharides have a degree of methoxylation above about 10%, above about 20%, above about 50%, above about 70%. In some embodiments, the acid oligosaccharides have a degree of methylation above about 10%, above about 20%, above about 50%, above about 70%.

Neutral oligosaccharides are saccharides which have a degree of polymerization of monose units exceeding 2, exceeding 3, exceeding 4, or exceeding 10, which are not or only partially digested in the intestine by the action of acids or digestive enzymes present in the human upper digestive tract (small intestine and stomach) but which are fermented by the human intestinal flora and preferably lack acidic groups. The neutral oligosaccharide is structurally (chemically) different from the acid oligosaccharide. The neutral oligosaccharides are saccharides which have a degree of polymerization of the oligosaccharide below 60 monose units. Monose units are sugar units having a closed ring structure e.g., the pyranose or furanose forms. In some embodiments, the neutral oligosaccharide comprises at least 90% or at least 95% monose units selected from the group consisting of mannose, arabinose, fructose, fucose, rhamnose, galactose, -D-galactopyranose, ribose, glucose, xylose and derivatives thereof, calculated on the total number of monose units contained therein. Suitable neutral oligosaccharides are preferably fermented by the gut flora. Non-limiting examples of suitable neutral oligosaccharides are cellobiose (4-O-β-D-glucopyranosyl-D-glucose), cellodextrins ((4-O-β-D-glucopyranosyl)n-D-glucose), β-cyclo-dextrins (Cyclic molecules of α-1-4-linked D-glucose; c-cyclodextrin-hexamer, β-cyclodextrin-heptamer and γ-cyclodextrin-octamer), indigestible dextrin, gentiooligosaccharides (mixture of β-1-6 linked glucose residues, some 1-4 linkages), glucooligosaccharides (mixture of α-D-glucose), isomaltooligosaccharides (linear α-1-6 linked glucose residues with some 1-4 linkages), isomaltose (6-O-α-D-glucopyranosyl-D-glucose); isomaltriose (6-O-α-D-glucopyranosyl-(1-6)-α-D-glucopyranosyl-D-glucose), panose (6-O-α-D-glucopyranosyl-(1-6)-α-D-glucopyranosyl-(1-4)-D-glucose), leucrose (5-O-α-D-glucopyranosyl-D-fructopyranoside), palatinose or isomaltulose (6-O-α-D-glucopyranosyl-D-fructose), theanderose (O-α-D-glucopyranosyl-(1-6)-O-α-D-glucopyranosyl-(1-2)-B-D-fructo furanoside), D-agatose, D-lyxo-hexylose, lactosucrose (O-β-D-galactopyranosyl-(1-4)-O-α-D-glucopyranosyl-(1-2)-β-D-fructofuranoside), o-galactooligosaccharides including raffinose, stachyose and other soy oligosaccharides (O-α-D-galactopyranosyl-(1-6)-α-D-glucopyranosyl-β-D-fructofuranoside), β-galactooligosaccharides or transgalacto-oligosaccharides (β-D-galactopyranosyl-(1-6)-[β-D-glucopyranosyl]ₙ-(1-4) α-D glucose), lactulose (4-O-β-D-galactopyranosyl-D-fructose), 4′-galactosyllactose (β-D-galactopyranosyl-(1-4)-O-β-D-glucopyranosyl-(1-4)-D-glucopyranose), synthetic galactooligosaccharide (neogalactobiose, isogalactobiose, galsucrose, isolactose I, II and III), fructans-Levan-type (β-D-(2→6)-fructofuranosyl)n α-D-glucopyranoside), fructans-Inulin-type (β-D-((2→1)-fructofuranosyl)n α-D-glucopyranoside), 1 f-β-fructofuranosylnystose (β-D-((2→4)-fructofuranosyl)n B-D-fructofuranoside), xylooligo-saccharides (B-D-((1→4)-xylose)n, lafinose, lactosucrose and arabinooligosaccharides.

In some embodiments, the neutral oligosaccharide is selected from the group consisting of fructans, fructooligosaccharides, indigestible dextrins galactooligo-saccharides (including transgalactooligosaccharides), xylooligosaccharides, arabinooligo-saccharides, glucooligosaccharides, mannooligosaccharides, fucooligosaccharides and mixtures thereof.

Suitable oligosaccharides and their production methods are further described in Laere K. J. M. (Laere, K. J. M., Degradation of structurally different non-digestible oligosaccharides by intestinal bacteria: glycosylhydrolases of Bi. adolescentis. PhD-thesis (2000), Wageningen Agricultural University, Wageningen, The Netherlands), the entire content of which is hereby incorporated by reference. Transgalactooligosaccharides (TOS) are for example sold under the trademark Vivinal™ (Borculo Domo Ingredients, Netherlands). Indigestible dextrin, which can be produced by pyrolysis of corn starch, comprises α(1→4) and α(1→6) glucosidic bonds, as are present in the native starch, and contains 1→2 and 1→3 linkages and levoglucosan. Due to these structural characteristics, indigestible dextrin contains well-developed, branched particles that are partially hydrolyzed by human digestive enzymes. Numerous other commercial sources of indigestible oligosaccharides are readily available and known to skilled persons in the art. For example, transgalactooligosaccharide is available from Yakult Honsha Co., Tokyo, Japan. Soybean oligosaccharide is available from Calpis Corporation distributed by Ajinomoto USA. Inc., Teaneck, N.J.

In a further embodiment, the prebiotic mixture of the pharmaceutical composition described herein comprises an acid oligosaccharide with a DP between 1 and 5000, prepared from pectin, alginate, and mixtures thereof, and a neutral oligosaccharide, selected from the group of fructans, fructooligosaccharides, indigestible dextrins, galactooligosaccharides including transgalacto-oligosaccharides, xylooligosaccharides, arabinooligosaccharides, glucooligosaccharides, manno-oligosaccharides, fucooligosaccharides, and mixtures thereof.

In certain embodiments, the prebiotic mixture comprises xylose. In other embodiments, the prebiotic mixture comprises a xylose polymer (i.e. xylan). In some embodiments, the prebiotic comprises xylose derivatives, such as xylitol, a sugar alcohol generated by reduction of xylose by catalytic hydrogenation of xylose, and also xylose oligomers (e.g., xylooligosaccharide). While xylose can be digested by humans, via xylosyltransferase activity, most xylose ingested by humans is excreted in urine. In contrast, some microorganisms are efficient at xylose metabolism or can be selected for enhanced xylose metabolism. Microbial xylose metabolism can occur by at least four pathways, including the isomerase pathway, the Weimburg pathway, the Dahms pathway, and, for eukaryotic microorganisms, the oxido-reductase pathway.

The xylose isomerase pathway involves the direct conversion of D-xylose into D-xylulose by xylose isomerase, after which D-xylulose is phosphorylated by xylulose kinase to yield D-xylolose-5-phosphate, an intermediate of the pentose phosphate pathway.

In the Weimberg pathway, D-xylose is oxidized to D-xylono-lactone by a D-xylose dehydrogenase. Then D-xylose dehydrogenase is hydrolyzed by a lactonase to yield D-xylonic acid, and xylonate dehydratase activity then yields 2-keto-3-deoxy-xylonate. The final steps of the Weimberg pathway are a dehydratase reaction to form 2-keto glutarate semialdehyde and an oxidizing reaction to form 2-ketoglutarate, an intermediate of the Krebs cycle.

The Dahms pathway follows the same mechanism as the Weimberg pathway but diverges once it has yielded 2-keto-3-deoxy-xylonate. In the Dahms pathway, an aldolase splits 2-keto-3-deoxy-xylonate into pyruvate and glycolaldehyde.

The xylose oxido-reductase pathway, also known as the xylose reductase-xylitol dehydrogenase pathway, begins by the reduction of D-xylose to xylitol by xylose reductase followed by the oxidation of xylitol to D-xylulose by xylitol dehydrogenase. As in the isomerase pathway, the next step in the oxido-reductase pathway is the phosphorylation of D-xylulose by xylulose kinase to yield D-xylolose-5-phosphate.

Xylose is present in foods like fruits and vegetables and other plants such as trees for wood and pulp production. Thus, xylose can be obtained in the extracts of such plants. Xylose can be obtained from various plant sources using known processes including acid hydrolysis followed by various types of chromatography. Examples of such methods to produce xylose include those described in Maurelli, L. et al. (2013), Appl. Biochem. Biotechnol. 170:1104-1118; Hooi H. T et al. (2013), Appl. Biochem. Biotechnol. 170:1602-1613; Zhang H-J. et al. (2014), Bioprocess Biosyst. Eng. 37:2425-2436.

### Culture and Storage of Consortium Constituents

For banking, the species included in the bacterial composition can be (1) isolated directly from a specimen or taken from a banked stock, (2) optionally cultured on a nutrient agar or broth that supports growth to generate viable biomass, and (3) the biomass optionally preserved in multiple aliquots in long-term storage.

In embodiments using a culturing step, the agar or broth contains nutrients that provide essential elements and specific factors that enable growth. An example would be a medium composed of 20 g/L glucose, 10 g/L yeast extract, 10 g/L soy peptone, 2 g/L citric acid, 1.5 g/L sodium phosphate monobasic, 100 mg/L ferric ammonium citrate, 80 mg/L magnesium sulfate, 10 mg/L hemin chloride, 2 mg/L calcium chloride, and 1 mg/L menadione. A variety of microbiological media and variations are well known in the art (e.g. R. M. Atlas, Handbook of Microbiological Media (2010) CRC Press). Medium can be added to the culture at the start, can be added during the culture, or can be intermittently/continuously flowed through the culture. The species in the bacterial composition can be cultivated alone, as a subset of the bacterial composition, or as an entire collection comprising the bacterial composition. As an example, a first strain can be cultivated together with a second strain in a mixed continuous culture, at a dilution rate lower than the maximum growth rate of either cell to prevent the culture from washing out of the cultivation.

The inoculated culture is incubated under favorable conditions for a time sufficient to build biomass. For bacterial compositions for human use this is often at normal body temperature (37° C.), pH, and other parameter with values similar to the normal human niche. The environment can be actively controlled, passively controlled (e.g., via buffers), or allowed to drift. For example, for anaerobic bacterial compositions (e.g., gut microbiota), an anoxic/reducing environment can be employed. This can be accomplished by addition of reducing agents/factors such as cysteine to the broth, and/or stripping it of oxygen. As an example, a culture of a bacterial composition can be grown at 37° C., pH 7, in the medium above, pre-reduced with 1 g/L cysteine.HCl.

When the culture has generated sufficient biomass, it can be preserved for banking or storage. The organisms can be placed into a chemical milieu that protects from freezing (adding ‘cryoprotectants’), drying (‘lyoprotectants’), and/or osmotic shock (‘osmoprotectants’), dispensing into multiple (optionally identical) containers to create a uniform bank, and then treating the culture for preservation. Containers are generally impermeable and have closures that assure isolation from the environment. Cryopreservation treatment is accomplished by freezing a liquid at ultra-low temperatures (e.g., at or below −80° C.). Dried preservation removes water from the culture by evaporation (in the case of spray drying or ‘cool drying’) or by sublimation (e.g., for freeze drying, spray freeze drying). Removal of water improves long-term bacterial composition storage stability at temperatures elevated above cryogenic. If the bacterial composition comprises spore forming species and results in the production of spores, the final composition can be purified by additional means such as density gradient centrifugation preserved using the techniques described above. Bacterial composition banking can be done by culturing and preserving the species individually, or by mixing the species together to create a combined bank. As an example of cryopreservation, a bacterial composition culture can be harvested by centrifugation to pellet the cells from the culture medium, the supernate decanted and replaced with fresh culture broth containing 15% glycerol. The culture can then be aliquoted into 1 mL cryotubes, sealed, and placed at −80° C. for long-term viability retention. This procedure achieves acceptable viability upon recovery from frozen storage.

Organism production can be conducted using similar culture steps to banking, including medium composition and culture conditions. It can be conducted at larger scales of operation, especially for clinical development or commercial production. At larger scales, there can be several subcultivations of the bacterial composition prior to the final cultivation. At the end of cultivation, the culture is harvested to enable further formulation into a dosage form for administration. This can involve concentration, removal of undesirable medium components, and/or introduction into a chemical milieu that preserves the bacterial composition and renders it acceptable for administration via the chosen route. For example, a bacterial composition can be cultivated to a concentration of 10¹⁰ CFU/mL, then concentrated 20-fold by tangential flow microfiltration; the spent medium may be exchanged by diafiltering with a preservative medium consisting of 2% gelatin, 100 mM trehalose, and 10 mM sodium phosphate buffer. The suspension can then be freeze-dried to a powder and titrated.

After drying, the powder can be blended to an appropriate potency, and mixed with other cultures and/or a filler such as microcrystalline cellulose for consistency and ease of handling, and the bacterial composition formulated as provided herein.

In one embodiment, a composition comprising a microbial consortium as described herein, is not a fecal transplant. In some embodiments all or essentially all of the bacterial entities present in a purified population are originally obtained from a fecal material and subsequently, e.g., for production of pharmaceutical compositions, are grown in culture as described herein or otherwise known in the art. In one embodiment, the bacterial cells are cultured from a bacterial stock and purified as described herein. In one embodiment, each of the populations of bacterial cells are independently cultured and purified, e.g., each population is cultured separately and subsequently mixed together. In one embodiment, one or more of the populations of bacterial cells in the composition are co-cultured

### Dosage, Administration and Formulations

In some embodiments, cells over a range of, for example, 2-5×10⁵, or more, e.g., 1×10⁶, 1×10⁷, 1×10⁸, 5×10⁸, 1×10⁹, 5×10⁹, 1×10¹⁰, 5×10¹⁰ or more can be administered in a composition comprising a microbial consortium. The dosage range for the bacteria depends upon the potency, and include amounts large enough to produce the desired effect, e.g., reduction in at least one symptom of a food allergy in a treated subject. The dosage should not be so large as to cause unacceptable adverse side effects. Generally, the dosage will vary with the type of illness, and with the age, condition, and sex of the patient. The dosage can be determined by one of skill in the art and can also be adjusted by the individual physician in the event of any complication.

For use in the various aspects described herein, an effective amount of cells in a composition as described herein comprises at least 10² bacterial cells, at least 1×10³ bacterial cells, at least 1×10⁴ bacterial cells, at least 1×10⁵ bacterial cells, at least 1×10⁶ bacterial cells, at least 1×10⁷ bacterial cells, at least 1×10⁸ bacterial cells, at least 1×10⁹ bacterial cells, at least 1×10¹⁰ bacterial cells, at least 1×10¹¹ bacterial cells, at least 1×10¹² bacterial cells or more. Where a microbial consortium is isolated and/or purified from a subject that is tolerant to a selected food allergen, the bacterial cells can be derived from one or more donors, or can be obtained from an autologous source. In some embodiments of the aspects described herein, the cells of the microbial consortium are expanded or maintained in culture prior to administration to a subject in need thereof. In one embodiment, the microbial consortium is obtained from a microbe bank. Members of a therapeutic or preventive/prophylactic consortium are generally administered together, e.g., in a single admixture. However, it is specifically contemplated herein that members of a given consortium can be administered as separate dosage forms or sub-mixtures or sub-combinations of the consortium members. Thus, for a consortium of e.g., six members, the consortium can be administered, for example, as a single preparation including all six members (in one or more dosage units, e.g., one or more capsules) or as two or more separate preparations that, in sum, include all members of the given consortium. While administration as a single admixture is preferred, a potential advantage of the use of e.g., individual units for each member of a consortium, is that the actual species administered to any given subject can be tailored, if necessary, by selecting the appropriate combination of, for example, single species dosage units that together comprise the desired consortium.

Biomass of administered species, per dose, vs. known in vivo biomass: It is contemplated herein that the consortium composition is formulated to deliver a larger biomass than the normal biomass of the commensal organisms in a “healthy” individual. For example, the range of biomasses contemplated for delivery and colonization can be found in TABLE 1, column 2, as compared to the normal biomass in a healthy individual as shown in TABLE 1, columns 3 & 4. The table below shows the range of administered biomasses of organisms relative to published data at specific locations. Note, in many cases the bacterial quantitation in Gustafsson, 1982 was to general categories of organisms, such as Clostridia, and incorporated multiple species under those headers. Individual species in the consortia would thus likely be less than the actual highest reported biomass at the specific locations; the small and large intestinal biomass data should thus be considered an upper-bound for what might occur in vivo in normal individuals.

TABLE 1
Consortia biomass compared with the biomass of the commensal
organisms in a healthy individual

| | | Small | Large |
|---|---|---|---|
| | Consortia | Intestinal | Intestinal |
| Species | Biomass | Biomass | Biomass |
| Bacteroides | 1 × 10⁷-5 × 10⁸ | <10³ CFU/g in | 10⁸-10¹¹ CFU/g |
| fragilis | CFU/mL | duodenum-jejunum | |
| | | 10³-10⁸ CFU/g | |
| | | in ileum | |
| B. | 1 × 10⁷-5 × 10⁸ | <10³ CFU/g in | 10⁸-10¹¹ CFU/g |
| thetaiotaomicron | CFU/mL | duodenum-jejunum | |
| | | 10³-10⁸ CFU/g | |
| | | in ileum | |
| B. ovatus | 1 × 10⁷-5 × 10⁸ | <10³ CFU/g in | 10⁸-10¹¹ CFU/g |
| | CFU/mL | duodenum-jejunum | |
| | | 10³-10⁸ CFU/g | |
| | | in ileum | |
| B. vulgatus | 1 × 10⁷-5 × 10⁸ | <10³ CFU/g in | 10⁸-10¹¹ CFU/g |
| | CFU/mL | duodenum-jejunum | |
| | | 10³-10⁸ CFU/g | |
| | | in ileum | |
| P. distasonis | 1 × 10⁷-5 × 10⁸ | <10³ CFU/g in | 0-1 × 10⁸ |
| | CFU/mL | duodenum-jejunum | CFU/mL |
| | | 10³-10⁸ CFU/g | |
| | | in ileum | |
| P. | 1 × 10⁷-5 × 10⁵ | <10³ CFU/g in | 0-1 × 10⁶ |
| melaninogenica | CFU/mL | oral cavity | CFU/mL |
| C. bifermentans | 1 × 10⁷-5 × 10⁸ | <10³ CFU/g in | 0-10⁶ CFU/g |
| | CFU/mL | duodenum-jejunum | |
| | | 10²-10⁴ CFU/g | |
| | | in ileum | |
| C. hiranonsis | 1 × 10⁷-5 × 10⁸ | <10³ CFU/g in | 0-10⁶ CFU/g |
| | CFU/mL | duodenum-jejunum | |
| | | 10²-10⁴ CFU/g | |
| | | in ileum | |
| C. leptum | 1 × 10⁷-5 × 10⁸ | <10³ CFU/g in | 0-10⁶ CFU/g |
| | CFU/mL | duodenum-jejunum | |
| | | 10²-10⁴ CFU/g | |
| | | in ileum | |
| Clostridium | 1 × 10⁷-5 × 10⁸ | <10 CFU/g in | 0-10⁶ CFU/g |
| ramosum | CFU/mL | duodenum-jejunum | |
| | | 10²-10⁴ CFU/g | |
| | | in ileum | |
| C. sardiniensis | 1 × 10⁷-5 × 108 | <10³ CFU/g in | 0-10⁶ CFU/g |
| | CFU/mL | duodenum-jejunum | |
| | | 10²-10⁴ CFU/g | |
| | | in ileum | |
| C. scindens | 1 × 10⁷-5 × 10⁸ | <10 CFU/g in | 0-10⁶ CFU/g |
| | CFU/mL | duodenum-jejunum | |
| | | 10²-10⁴ CFU/g | |
| | | in ileum | |
| Parabacteroides | 1 × 10⁷-5 × 10⁸ | <10³ CFU/g in | 0-10⁶ CFU/g |
| goldsteinii | CFU/mL | duodenum-jejunum | |
| | | 10³-10⁸ CFU/g | |
| | | in ileum | |
| Prevotella | 1 × 10⁷-5 × 10⁸ | <10³ CFU/g in | 0-10⁶ CFU/g |
| tannerae | CFU/mL | duodenum-jejunum | |
| | | <10⁴ CFU/g in | |
| | | ileum | |

A pharmaceutical composition comprising a microbial consortium can be administered by any method suitable for depositing in the gastrointestinal tract, preferably the colon, of a subject (e.g., human, mammal, animal, etc.). Examples of routes of administration include rectal administration by colonoscopy, suppository, enema, upper endoscopy, or upper push enteroscopy. Additionally, intubation through the nose or the mouth by nasogastric tube, nasoenteric tube, or nasal jejunal tube can be utilized. Oral administration by a solid such as a pill, tablet, a suspension, a gel, a geltab, a semisolid, a tablet, a sachet, a lozenge or a capsule or microcapsule, or as an enteral formulation, or re-formulated for final delivery as a liquid, a suspension, a gel, a geltab, a semisolid, a tablet, a sachet, a lozenge or a capsule, or as an enteral formulation can be utilized as well. Also contemplated herein are food items that are inoculated with a microbial consortium as described herein. Compositions can also be treated or untreated fecal flora, entire (or substantially entire) microbiota, or partially, substantially or completely isolated or purified fecal flora, and can be lyophilized, freeze-dried or frozen, or processed into a powder.

In some embodiments, the compositions described herein can be administered in a form containing one or more pharmaceutically acceptable carriers. Suitable carriers are well known in the art and vary with the desired form and mode of administration of the composition. For example, pharmaceutically acceptable carriers can include diluents or excipients such as fillers, binders, wetting agents, disintegrators, surface-active agents, glidants, lubricants, and the like. Typically, the carrier may be a solid (including powder), liquid, or combinations thereof. Each carrier is preferably “acceptable” in the sense of being compatible with the other ingredients in the composition and not injurious to the subject. The carrier may be biologically acceptable and inert (e.g., it permits the composition to maintain viability of the biological material until delivered to the appropriate site).

Oral compositions can include an inert diluent or an edible carrier. For the purpose of oral therapeutic administration, the active compound can be incorporated with excipients and used in the form of tablets, lozenges, pastilles, troches, or capsules, e.g., gelatin capsules. Oral compositions can also be prepared by combining a composition of the present disclosure with a food. In one embodiment a food used for administration is chilled, for instance, iced flavored water. In certain embodiments, the food item is not a potentially allergenic food item (e.g., not soy, wheat, peanut, tree nuts, dairy, eggs, shellfish or fish). Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, orange flavoring, or other suitable flavorings. These are for purposes of example only and are not intended to be limiting.

The compositions comprising a microbial consortium can also be prepared in the form of suppositories (e.g., with conventional suppository bases such as cocoa butter and other glycerides) or retention enemas for rectal delivery. The compositions can be prepared with carriers that will protect the consortium against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from, for instance, Alza Corporation and Nova Pharmaceuticals, Inc. Liposomal suspensions can also be used as pharmaceutically acceptable carriers. These can be prepared according to methods known to those skilled in the art.

In some embodiments, a composition can be encapsulated (e.g., enteric coated formulations). For instance, when the composition is to be administered orally, the dosage form is formulated so the composition is not exposed to conditions prevalent in the gastrointestinal tract before the small intestine, e.g., high acidity and digestive enzymes present in the stomach, such a formulation can provide delivery of viable bacteria to the small intestine. The encapsulation of compositions for therapeutic use is routine in the art. Encapsulation can include hard-shelled capsules, which can be used for dry, powdered ingredients soft-shelled capsules. Capsules can be made from aqueous solutions of gelling agents such as animal protein (e.g., gelatin), plant polysaccharides or derivatives like carrageenans and modified forms of starch and cellulose. Other ingredients can be added to a gelling agent solution such as plasticizers (e.g., glycerin and/or sorbitol), coloring agents, preservatives, disintegrants, lubricants and surface treatment.

In one embodiment, a microbial consortium as described herein is formulated with an enteric coating. An enteric coating can control the location of where a microbial consortium is released in the digestive system. Thus, an enteric coating can be used such that a microbial consortium-containing composition does not dissolve and release the microbes in the stomach, which can be a toxic environment for many microbes, but rather travels to the small intestine, where it dissolves and releases the microbes in an environment where they can survive. An enteric coating can be stable at low pH (such as in the stomach) and can dissolve at higher pH (for example, in the small intestine). Thus, enteric coating formulations can also permit delivery of viable bacteria to the small intestine. Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac (trade name EmCoat 120 N, Marcoat 125); cellulose acetate phthalate (trade names AQUACOAT™, AQUACOAT ECD™, SEPIFILM™, KLUCEL™, and METOLOSE™); polyvinylacetate phthalate (trade name SURETERIC™); and methacrylic acid (trade name EUDRAGIT™). In one embodiment, an enteric coated probiotic composition comprising members of a microbial consortium as described herein is administered to a subject. In another embodiment, an enteric coated probiotic and prebiotic composition is administered to a subject.

Formulations suitable for rectal administration include gels, creams, lotions, aqueous or oily suspensions, dispersible powders or granules, emulsions, dissolvable solid materials, douches, and the like. The formulations are preferably provided as unit-dose suppositories comprising the active ingredient in one or more solid carriers forming the suppository base, for example, cocoa butter. Suitable carriers for such formulations include petroleum jelly, lanolin, polyethyleneglycols, alcohols, and combinations thereof. Alternatively, colonic washes with the rapid recolonization deployment agent of the present disclosure can be formulated for colonic or rectal administration.

Formulations suitable for oral administration may be provided as discrete units, such as tablets, capsules, cachets, syrups, elixirs, prepared food items, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

In some embodiments, the microbial consortium can be formulated in a food item. Some non-limiting examples of food items to be used with the methods and compositions described herein include: popsicles, cheeses, creams, chocolates, milk, meat, drinks, yogurt, pickled vegetables, kefir, miso, sauerkraut, etc. In other embodiments, the food items can be juices, refreshing beverages, tea beverages, drink preparations, jelly beverages, and functional beverages; alcoholic beverages such as beers; carbohydrate-containing foods such as rice food products, noodles, breads, and pastas; paste products such as fish, hams, sausages, paste products of seafood; retort pouch products such as curries, food dressed with a thick starchy sauce, and Chinese soups; soups, dairy products such as milk, dairy beverages, ice creams, cheeses, and yogurts; fermented products such as fermented soybean pastes, fermented beverages, and pickles; bean products; various confectionery products including biscuits, cookies, and the like, candies, chewing gums, gummies, cold desserts including jellies, cream caramels, and frozen desserts; instant foods such as instant soups and instant soy-bean soups; and the like. It is preferred that food preparations not require cooking after admixture with the microbial consortium to avoid killing the microbes.

Formulations of a microbial consortium can be prepared by any suitable method, typically by uniformly and intimately admixing the consortium with liquids or finely divided solid carriers or both, in the required proportions and then, if necessary, shaping the resulting in mixture into the desired shape. In addition, the microbial consortium can be treated to prolong shelf-life, preferably the shelf-life of the pre-determined gut flora will be extended via freeze drying.

In some embodiments, the microbial consortium as described herein is combined with one or more additional probiotic organisms prior to treatment of a subject. As used herein, the a probiotic refers to microorganisms that form at least a part of the transient or endogenous flora or microbial consortium and thereby exhibit a beneficial prophylactic and/or therapeutic effect on the host organism. Probiotics are generally known to be clinically safe (i.e., non-pathogenic) by those individuals skilled in the art. Typical lactic acid-producing bacteria useful as a probiotic of this invention are efficient lactic acid producers which include non-pathogenic members of the Bacillus genus which produce bacteriocins or other compounds which inhibit the growth of pathogenic organisms.

Exemplary lactic acid-producing, non-pathogenic Bacillus species include, but are not limited to: Bacillus coagulans; Bacillus coagulans Hammer; and Bacillus brevis subspecies coagulans.

Exemplary lactic acid-producing Lactobacillus species include, but are not limited to: Lactobacillus acidophilus, Lactobacillus casei, Lactobacillus DDS-1, Lactobacillus GG, Lactobacillus rhamnosus, Lactobacillus plantarum, Lactobacillus reuteri, Lactobacillus gasserii, Lactobacillus jensenii, Lactobacillus delbruekii, Lactobacillus, bulgaricus, Lactobacillus salivarius and Lactobacillus sporogenes (also designated as Bacillus coagulans). Exemplary lactic acid-producing Sporolactobacillus species include all Sporolactobacillus species, for example, Sporolactobacillus P44.

Exemplary lactic acid-producing Bifidobacterium species include, but are not limited to: Bifidiobacterium adolescentis, Bifidiobacterium animalis, Bifidiobacterium bifidum, Bifidiobacterium bifidus, Bifidiobacterium breve, Bifidiobacterium infantis, Bifidiobacterium infantus, Bifidiobacterium longum, and any genetic variants thereof.

Examples of suitable non-lactic acid-producing Bacillus include, but are not limited to: Bacillus subtilis. Bacillus uniflagellatus, Bacillus lateropsorus, Bacillus laterosporus BOD, Bacillus megaterium, Bacillus polymyxa, Bacillus licheniformis. Bacillus pumilus, and Bacillus sterothermophilus. Other species that could be employed due to probiotic activity include members of the Streptococcus (Enterococcus) genus. For example, Enterococcus faecium, is commonly used as a livestock probiotic and, thus, could be utilized as a co-administration agent. Furthermore, it is also intended that any of the acid-producing species of probiotic or nutritional bacteria known in the art can be used in the compositions comprising a microbial consortium as described herein.

A nutrient supplement comprising the microbial consortium as described herein can include any of a variety of nutritional agents, including vitamins, minerals, essential and nonessential amino acids, carbohydrates, lipids, foodstuffs, dietary supplements, short chain fatty acids and the like. Preferred compositions comprise vitamins and/or minerals in any combination. Vitamins for use in a composition as described herein can include vitamins B, C, D, E, folic acid, K, niacin, and like vitamins. The composition can contain any or a variety of vitamins as may be deemed useful for a particularly application, and therefore, the vitamin content is not to be construed as limiting. Typical vitamins are those, for example, recommended for daily consumption and in the recommended daily amount (RDA), although precise amounts can vary. The composition can preferably include a complex of the RDA vitamins, minerals and trace minerals as well as those nutrients that have no established RDA, but have a beneficial role in healthy human or mammal physiology. The preferred mineral format would include those that are in either the gluconate or citrate form because these forms are more readily metabolized by lactic acid bacteria. In a related embodiment, the compositions described herein are contemplated to comprise a microbial consortium in combination with a viable lactic acid bacteria in combination with any material to be adsorbed, including but not limited to nutrient supplements, foodstuffs, vitamins, minerals, medicines, therapeutic compositions, antibiotics, hormones, steroids, and the like compounds where it is desirable to insure efficient and healthy absorption of materials from the gastrointestinal tract into the blood. The amount of material included in the composition can vary widely depending upon the material and the intended purpose for its absorption, such that the composition is not to be considered as limiting.

In some embodiments, the compositions described herein can further include a prebiotic and/or a fiber. Many forms of fiber exhibit some level of prebiotic effect. Thus, there is considerable overlap between substances that can be classified as “prebiotics” and those that can be classified as “fibers”. Non-limiting examples of prebiotics suitable for use in the compositions and methods include psyllium, fructo-oligosaccharides, inulin, oligofructose, galacto-oligosaccharides, isomalto-oligosaccharides xylo-oligosaccharides, soy-oligosaccharides, gluco-oligosaccharides, mannan-oligosaccharides, arabinogalactan, arabinxylan, lacto sucrose, gluconannan, lactulose, polydextrose, oligodextran, gentioligosaccharide, pectic oligosaccharide, xanthan gum, gum arabic, hemicellulose, resistant starch and its derivatives, and mixtures and/or combinations thereof. The compositions can comprise from about 100 mg to about 100 g, alternatively from about 500 mg to about 50 g, and alternatively from about 1 g to about 40 g, of probiotic, per day or on a less than daily schedule.

Aspects of the technology described herein also include short chain fatty acids (SCFAs) and medium chain triglycerides (MCTs). Short chain fatty acids can have immunomodulatory (i.e., immunosuppressive) effects and therefore their production (i.e., biosynthesis or conversion by fermentation) is advantageous for the prevention, control, mitigation, and treatment of autoimmune and/or inflammatory disorders (Lara-Villoslada F. et al, 2006. Eur J Nutr. 45(7): 418-425). In germ-free mice and vancomycin-treated conventional mice, administration of SCFA (acetate, propionate, or butyrate) restored normal numbers of T_{regs} in the large intestine (Smith P M, et al. Science. 2013; 569-573). Short-chain fatty acids (SCFA) are produced by some bacteria as a byproduct of xylose fermentation. SCFA are one of the most abundant metabolites produced by the gut microbiome, particularly the family Clostridiaceae, including members of the genus Clostridium, Ruminococcus, or Blautia. In some aspects, the pharmaceutical composition, dosage form, or kit comprises at least one type of microbe (e.g., one or more microbial species, such as a bacterial species, or more than one strain of a particular microbial species) and at least one type of prebiotic such that the composition, dosage form, or kit is capable of increasing the level of one or more immunomodulatory SCFA (e.g., acetate, propionate, butyrate, or valerate) in a mammalian subject. Optionally, the pharmaceutical composition, dosage form, or kit further comprises one or more substrates of one or more SCFA-producing fermentation and/or biosynthesis pathways. In certain embodiments, the administration of the composition, dosage form, or kit to a mammalian subject results in the increase of one or more SCFAs in the mammalian subject by approximately 1.5-fold, 2-fold, 5-fold, 10-fold, 20-fold, 50-fold, 100-fold, or greater than 100-fold. In some embodiments, the dysbiosis is caused by a deficiency in microbes that produce short chain fatty acids. Accordingly, in some embodiments, the probiotic composition can contain a species of bacteria that produce short chain fatty acids.

MCTs passively diffuse from the GI tract to the portal system (longer fatty acids are absorbed into the lymphatic system) without requirement for modification like long-chain fatty acids or very-long-chain fatty acids. In addition, MCTs do not require bile salts for digestion. Patients who have malnutrition or malabsorption syndromes are treated with MCTs because they do not require energy for absorption, use, or storage. Medium-chain triglycerides are generally considered a good biologically inert source of energy that the human body finds reasonably easy to metabolize. They have potentially beneficial attributes in protein metabolism, but may be contraindicated in some situations due to their tendency to induce ketogenesis and metabolic acidosis. Due to their ability to be absorbed rapidly by the body, medium-chain triglycerides have found use in the treatment of a variety of malabsorption ailments. MCT supplementation with a low-fat diet has been described as the cornerstone of treatment for primary intestinal lymphangiectasia (Waldmann's disease). MCTs are an ingredient in parenteral nutritional emulsions.

Also contemplated herein are kits comprising, at a minimum, a microbial consortium prep or formulations comprising all of the members of the consortium in an admixture or comprising all of the members of the consortium in sub-combinations or sub-mixtures. In some embodiments, the kit further comprises empty capsules to be filled by the practitioner and/or one or more reagents for enteric coating such capsules. It is also contemplated herein that the microbe preparation is provided in a dried, lyophilized or powdered form.

In one embodiment, a kit comprises at least two species selected from the group consisting of: Bacteroides fragilis, Bacteroides ovatus, Bacteroides vulgatus, Parabacteroides distasonis and Prevotella melaninogenica. In another embodiment a kit comprises at least three, at least four, at least five, or all six of the species form the group of: Clostridium ramosum, Clostridium scindens, Clostridium hiranonsis, Clostridium bifermentans, Clostridium leptum, and Clostridium sardiniensis.

In another embodiment, the kits comprise at least two, at least three, at least four, or all five species selected from the group consisting of: Bacteroides fragilis, Bacteroides ovatus, Bacteroides vulgatus, Parabacteroides distasonis, Prevotella melaninogenica, and at least one, at least two, at least three, at least four, at least five, or all six species selected from the group consisting of: Clostridium ramosum, Clostridium scindens, Clostridium hiranonsis, Clostridium bifermentans, Clostridium leptum, and Clostridium sardiniensis. In another embodiment, the kit comprises at least one reducing agent such as N-acetylcysteine, cysteine, or methylene blue for growing, maintaining and/or encapsulating the microbes under anaerobic conditions. The kits described herein are also contemplated to include cell growth media and supplements necessary for expanding the microbial preparation. The kits described herein are also contemplated to include one or more prebiotics as described herein.

Prior to administration of the bacterial composition, the patient may optionally have a pretreatment protocol to prepare the gastrointestinal tract to receive the bacterial composition. In certain embodiments, the pretreatment protocol is advisable, such as when a patient has an acute infection with a highly resilient pathogen. In other embodiments, the pretreatment protocol is entirely optional, such as when the pathogen causing the infection is not resilient, or the patient has had an acute infection that has been successfully treated but where the physician is concerned that the infection may recur. In these instances, the pretreatment protocol can enhance the ability of the bacterial composition to affect the patient's microbiome. In an alternative embodiment, the subject is not pre-treated with an antibiotic.

As one way of preparing the patient for administration of the microbial ecosystem, at least one antibiotic can be administered to alter the bacteria in the patient. As another way of preparing the patient for administration of the microbial ecosystem, a standard colon-cleansing preparation can be administered to the patient to substantially empty the contents of the colon, such as used to prepare a patient for a colonoscopy. By “substantially emptying the contents of the colon,” this application means removing at least 75%, at least 80%, at least 90%, at least 95%, or about 100% of the contents of the ordinary volume of colon contents. Antibiotic treatment can precede the colon-cleansing protocol.

If a patient has received an antibiotic for treatment of an infection, or if a patient has received an antibiotic as part of a specific pretreatment protocol, in one embodiment the antibiotic should be stopped in sufficient time to allow the antibiotic to be substantially reduced in concentration in the gut before the bacterial composition is administered. In one embodiment, the antibiotic may be discontinued 1, 2, or 3 days before the administration of the bacterial composition. In one embodiment, the antibiotic can be discontinued 3, 4, 5, 6, or 7 antibiotic half-lives before administration of the bacterial composition. If the pretreatment protocol is part of treatment of an acute infection, the antibiotic may be chosen so that the infection is sensitive to the antibiotic, but the constituents in the bacterial composition are not sensitive to the antibiotic.

Any of the preparations described herein can be administered once on a single occasion or on multiple occasions, such as once a day for several days or more than once a day on the day of administration (including twice daily, three times daily, or up to five times daily). Or the preparation can be administered intermittently according to a set schedule, e.g., once weekly, once monthly, or when the patient relapses from the primary illness. In another embodiment, the preparation can be administered on a long-term basis to assure the maintenance of a protective or therapeutic effect.

In one embodiment, a first microbial consortium comprising at least two bacterial species known to enhance colonization of beneficial organisms (e.g., Bacteroides vulgatus and Bacteroides ovatus) is administered to a subject prior to administration of a second microbial consortium.

Another aspect described herein relates to a method for enhancing the colonization and/or persistence of a microbial consortium, the method comprising administering a first microbial consortium comprising at least two bacterial species selected from the group consisting of: Bacteroides fragilis, Bacteroides ovatus, Bacteroides vulgatus, Parabacteroides distasonis, Prevotella melaninogenica, to a subject prior to administering a second microbial consortium comprising at least 4 bacterial species selected from the group consisting of: Clostridium ramosum, C. scindens, C. hiranonsis, C. bifermentans, C. leptum and C. sardiniensis, wherein the first microbial consortium enhances the colonization and/or persistence of the second microbial consortium.

In this context, persistence is the maintenance of one or more members of the microbial consortium in the subject (e.g., the gastrointestinal tract) at a number, biomass or activity that is at or above the threshold for treating and/or preventing food allergy. Persistence can be measured by obtaining a stool sample to determine the number, biomass, and/or activity of one or more members of the microbial consortium. In some embodiments, persistence can be measured by obtaining a ratio of the measured biomass of at least two members of the microbial consortium in the stool sample.

It is also contemplated herein that a first microbial consortium comprising at least two bacterial species selected from the group consisting of: Clostridium ramosum, C. scindens, C. hiranonsis, C. bifermentans, C. leptum and C. sardiniensis, is administered to a subject prior to administering a second microbial consortium comprising at least two bacterial species selected from the group consisting of: Bacteroides fragilis, Bacteroides ovatus, Bacteroides vulgatus, Parabacteroides distasonis, Prevotella melaninogenica.

It is also contemplated herein that a first microbial consortium comprising at least two bacterial species selected from the group consisting of: Clostridium ramosum, C. scindens, C. hiranonsis, C. bifermentans, C. leptum and C. sardiniensis, is administered to a subject in combination with (e.g., simultaneously) a second microbial consortium comprising at least two bacterial species selected from the group consisting of: Bacteroides fragilis, Bacteroides ovatus, Bacteroides vulgatus, Parabacteroides distasonis, and Prevotella melaninogenica.

### Efficacy

Typically, an atopic response, e.g., a food allergy or other allergic response, among others, can manifest with one of more of the following symptoms or indicators: (i) a marked drop in core body temperature, (ii) an increase in total IgE, (iii) an increase in allergen-specific IgE, (iv) mast cell expansion, (v) release of mast cell granule protease 1 (MMCP-1) and (vi) increase in Tₕ2 cell skewing. Thus, efficacious treatment and/or prevention of food allergy using the methods and compositions described herein can reduce or eliminate at least one of the symptoms or indicators associated with food allergy, as described above. In this context, reduced symptoms or indicators mean at least 20% reduced, at least 30% reduced, at least 40% reduced, at least 50% reduced, at least 60% reduced, at least 70% reduced, at least 80% reduced, at least 90% reduced, at least 95% reduced, at least 98% reduced or even at least 99% or further reduction. Methods for the measurement of each of these parameters are known to those of ordinary skill in the art.

The methods and compositions described herein provide treatment or prevention of food allergy involving or provoking anaphylaxis—i.e., IgE-mediated histamine release or direct allergen-mediated degranulation of mast cells and basophils and resulting pathology. Non-limiting examples include allergy or anaphylactic reaction to peanut, tree nuts, and shellfish, among others noted elsewhere herein. Food sensitivity, e.g., lactose intolerance or gluten intolerance involves different mechanisms. While it is contemplated that a microbial consortium as described herein can benefit those with food sensitivities (e.g., by reducing or eliminating a dysbiotic state and thereby reducing gut inflammation), the distinction between food sensitivities and food allergies should be specifically noted. First and foremost, sensitivities do not provoke an anaphylactic response.

Effective prevention of food allergy can be assessed using an accepted animal model, such as that described herein or others known to those of ordinary skill in the art, wherein a regimen that sensitizes the animals to a given food allergen in the absence of microbial consortium treatment fails to provoke a substantial allergic response in animals administered a protective microbial consortium as described herein. In this context, the term “fails to provoke a substantial allergic response” means that there is less than 20% of the allergic response (as measured by one or more of the criteria (i)-(vi) described above) seen in animals sensitized to the allergen but without administration of a protective or therapeutic microbial consortium as described herein. In human clinical practice, prevention or cure can be evaluated by administration of the given microbial consortium followed by administration of an allergen under controlled circumstances in a doctor's office or hospital setting. For prevention, the microbial consortium can be administered prior to a patient's initial exposure to or consumption of a given food allergen. For therapy for established food allergy, the microbial consortium can be administered as described herein, followed by consumption of the food allergen in a controlled clinical setting. A lack of allergic reaction, or even a reduced allergic reaction relative to the patient's previous allergic responses to the allergen (i.e., at least 20% reduced, at least 30% reduced, at least 40% reduced, at least 50% reduced, at least 60% reduced, at least 70% reduced, at least 80% reduced, at least 90% reduced, at least 95% reduced, at least 98% reduced or even at least 99% or further reduction) is evidence of effective treatment.

Repeated administration of the microbial consortium may be beneficial to maintain a protective or curative effect.

In addition, efficacy of a particular formulation can be determined in vitro or in an in vivo or in situ mouse model as described in herein or as known in the art (e.g., Noval Rivas et al. J Allergy Clin Immunol (2013) 131(1):201-212 or Noval Rivas et al, Immunity (2015) 42:512-523, the contents of which are each incorporated herein in their entirety).

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al (eds), Molecular Biology and Biotechnology: a Comprehensive Desk Reference, published by YCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1 st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the methods and compositions described herein can be defined according to any of the following numbered paragraphs:
1) A pharmaceutical composition comprising:
   a. a preparation comprising a microbial consortium of isolated bacteria that comprises two to twenty species of viable gut bacteria, at least two of which are selected from the group consisting of: Bacteroides vulgatus, Parabacteroides distasonis, and Prevotella melaninogenica, in an amount sufficient to treat or prevent a dysbiosis when administered to an individual in need thereof, and
   b. a pharmaceutically acceptable carrier.
2) The pharmaceutical composition of paragraph 1, further comprising at least one of Bacteroides fragilis and Bacteroides ovatus.
3) The pharmaceutical composition of paragraph 1, further comprising Bacteroides fragilis and Bacteroides ovatus.
4) The pharmaceutical composition of paragraph 1, wherein the preparation comprises each of Bacteroides fragilis, Bacteroides ovatus, Bacteroides vulgatus, Parabacteroides distasonis, and Prevotella melaninogenica.
5) The pharmaceutical composition of any one of paragraphs 1-4, further comprising one or more of the bacteria in TABLE 4.
6) The pharmaceutical composition of any one of paragraphs 1-5, wherein the dysbiosis is associated with an inflammatory disease or a metabolic disorder.
7) The pharmaceutical composition of any one of paragraphs 1-6, wherein the dysbiosis is associated with an atopic disease or disorder.
8) The pharmaceutical composition of any one of paragraphs 1-7, wherein the viable gut bacteria are anaerobic gut bacteria.
9) The pharmaceutical composition of any one of paragraphs 1-8, formulated to deliver the viable bacteria to the small intestine.
10) The pharmaceutical composition of any one of paragraphs 1-9, wherein the pharmaceutically acceptable carrier comprises an enteric coating composition that encapsulates the microbial consortium.
11) The pharmaceutical composition of paragraph 10, wherein the enteric coating composition is in the form of a capsule, gel, pastille, tablet or pill.
12) The pharmaceutical composition of any one of paragraphs 1-11, wherein the viable gut bacteria are human gut bacteria.
13) The composition of any one of paragraphs 1-12, wherein the consortium comprises at least three species selected from the group consisting of: Bacteroides fragilis, Bacteroides ovatus, Bacteroides vulgatus, Parabacteroides distasonis, and Prevotella melaninogenica.
14) The pharmaceutical composition of any one of paragraphs 1-13, wherein the consortium comprises at least four species selected from the group consisting of Bacteroides fragilis, Bacteroides ovatus, Bacteroides vulgatus, Parabacteroides distasonis, and Prevotella melaninogenica.
15) The pharmaceutical composition of any one of paragraphs 1-14, wherein the consortium further comprises at least one species selected from the group consisting of: Clostridium ramosum, Clostridium scindens, Clostridium hiranonsis, Clostridium bifermentans, Clostridium leptum, and Clostridium sardiniensis.
16) The pharmaceutical composition of any one of paragraphs 1-15, wherein the consortium further comprises at least two species selected from the group consisting of: Clostridium ramosum, Clostridium scindens, Clostridium hiranonsis, Clostridium bifermentans, Clostridium leptum, and Clostridium sardiniensis.
17) The pharmaceutical composition of any one of paragraphs 1-16, wherein the consortium further comprises at least three species selected from the group consisting of: Clostridium ramosum, Clostridium scindens, Clostridium hiranonsis, Clostridium bifermentans, Clostridium leptum, and Clostridium sardiniensis.
18) The pharmaceutical composition of any one of paragraphs 1-17, wherein the consortium further comprises at least four species selected from the group consisting of: Clostridium ramosum, Clostridium scindens, Clostridium hiranonsis, Clostridium bifermentans, Clostridium leptum, and Clostridium sardiniensis.
19) The pharmaceutical composition of any one of paragraphs 1-18, wherein the consortium further comprises at least five species selected from the group consisting of Clostridium ramosum, Clostridium scindens, Clostridium hiranonsis, Clostridium bifermentans, Clostridium leptum, and Clostridium sardiniensis.
20) The pharmaceutical composition of any one of paragraphs 1-19, wherein the consortium further comprises each of the species Clostridium ramosum, Clostridium scindens, Clostridium hiranonsis, Clostridium bifermentans, Clostridium leptum, and Clostridium sardiniensis.
21) The pharmaceutical composition of any one of paragraphs 1-20, wherein the species of viable gut bacteria are present in substantially equal biomass.
22) The pharmaceutical composition of any one of paragraphs 1-21, wherein the composition is formulated to deliver a dose of at least 1×10⁹ colony forming units (CFUs).
23) The pharmaceutical composition of any one of paragraphs 1-22, wherein the composition is formulated to deliver at least 1×10⁹ CFUs in less than 30 capsules per one-time dose.
24) The pharmaceutical composition of any one of paragraphs 1-23, wherein the composition is frozen for storage.
25) The pharmaceutical composition of any one of paragraphs 1-24, wherein the species of viable gut bacteria are encapsulated under anaerobic conditions.
26) The pharmaceutical composition of paragraph 25, wherein anaerobic conditions comprise one or more of the following:
   a. oxygen impermeable capsules,
   b. addition of a reducing agent including N-acetylcysteine, cysteine, or methylene blue to the composition, or
   c. use of spores for organisms that sporulate
27) The pharmaceutical composition of any one of paragraphs 1-26, wherein the composition comprises at least two bacterial species, each comprising a 16S rDNA sequence at least 97% identical to a 16S rDNA sequence present in a reference strain operational taxonomic unit, the reference strains selected from the species Bacteroides vulgatus, Parabacteroides distasonis, and Prevotella melaninogenica
28) The pharmaceutical composition of any one of paragraphs 1-27, wherein the composition comprises at least three bacterial species, each comprising a 16S rDNA sequence at least 97% identical to a 16S rDNA sequence present in a reference strain operational taxonomic unit, the reference strains selected from the species Bacteroides vulgatus, Parabacteroides distasonis and Prevotella melaninogenica.
29) The pharmaceutical composition of any one of paragraphs 1-28, wherein the composition comprises at least four bacterial species, each comprising a 16S rDNA sequence at least 97% identical to a 16S rDNA sequence present in a reference strain operational taxonomic unit, the reference strains selected from the species Bacteroides fragilis, Bacteroides ovatus, Bacteroides vulgatus, Parabacteroides distasonis, and Prevotella melaninogenica.
30) The pharmaceutical composition of any one of paragraphs 1-29, wherein the composition comprises at least five bacterial species, each comprising a 16S rDNA sequence at least 97% identical to a 16S rDNA sequence present in a reference strain operational taxonomic unit, the reference strains including each of the species Bacteroides fragilis, Bacteroides ovatus, Bacteroides vulgatus, Parabacteroides distasonis, and Prevotella melaninogenica.
31) The pharmaceutical composition of any one of paragraphs 1-30, wherein the composition does not comprise any of the Species Escherichia coli, Klebsiella pneumoniae, Proteus mirabilis, Enterobacter cloacae, Bilophila wadsworthia, Alistipes onderdonkii, Desulfovibrio species, Lactobacillus johnsonii, or Parasutterella excrementihominis.
32) The pharmaceutical composition of any one of paragraphs 1-31, wherein the composition does not comprise bacteria of the Genera Bilophila, Enterobacter, Escherichia, Klebsiella, Proteus, Alistipes, Blautia, Desulfovibrio, and Parasutterella.
33) The pharmaceutical composition of any one of paragraphs 1-32, wherein the composition does not comprise bacteria of the Families Desulfovibrionaceae, Enterobacteriaceae, Rikenellaceae, and Sutterellaceae.
34) The pharmaceutical composition of any one of paragraphs 1-33, wherein the composition does not comprise bacteria of the Families Laciobacillaceae, or Enterbacteriaceae.
35) The pharmaceutical composition of any one of paragraphs 1-34, wherein the composition does not comprise bacteria of the Order Burkholdales, Desulfovibrionales, or Enterobacteriales.
36) The pharmaceutical composition of paragraph 1, which comprises at least four species of viable non-pathogenic gut bacteria.
37) The pharmaceutical composition of any one of paragraphs 1-36, which comprises at least two and up to eleven species of viable non-pathogenic gut bacteria.
38) The pharmaceutical composition of any one of paragraphs 1-37, wherein the microbial consortium comprises Bacteroides fragilis, Bacteroides ovatus, Bacteroides vulgatus, Parabacteroides distasonis, Prevotella melaninogenica, Clostridium ramosum, Clostridium scindens, Clostridium rhiranonsis, Clostridium bifermentans, Clostridium lepturn, and Clostridium sardiniensis
39) The pharmaceutical composition of any one of paragraphs 1-35 or paragraph 37, wherein the consortium consists essentially of: Bacteroides vulgatus, Parabacteroides distasonis, and Prevotella melaninogenica.
40) The pharmaceutical composition of any one of paragraphs 1-39, wherein the consortium consists essentially of Bacteroides fragilis, Bacteroides ovatus, Bacteroides vulgatus, Parabacteroides distasonis, and Prevotella melaninogenica.
41) The pharmaceutical composition of any one of paragraphs 1-40, wherein the consortium consists essentially of: Clostridium ramosum, Clostridium scindens, Clostridium hiranonsis, Clostridium bifermentans, Clostridium lepturn, Clostridium sardiniensis, Bacteroides fragilis, Bacteroides ovatus, Bacteroides vulgatus, Parabacteroides distasonis, and Prevotella melaninogenica.
42) The pharmaceutical composition of paragraph 10 or paragraph 11, wherein the enteric coating comprises a polymer, nanoparticle, fatty acid, shellac, or a plant fiber.
43) The pharmaceutical composition of any one of paragraphs 1-42, wherein the microbial consortium is encapsulated, lyophilized, formulated in a food item, or is formulated as a liquid, gel, fluid-gel, or nanoparticles in a liquid.
44) The pharmaceutical composition of any one of paragraphs 1-43, further comprising a prebiotic composition.
45) A pharmaceutical composition comprising:
   a. a preparation comprising at least two species of isolated, viable, anaerobic gut bacteria selected from the group consisting of: Bacteroides vulgatus, Parabacteroides distasonis, and Prevotella melaninogenica, in an amount sufficient to treat or prevent dysbiosis when administered to an individual in need thereof, and
   b. a pharmaceutically acceptable carrier.
46) A pharmaceutical composition comprising:
   a. a preparation comprising at least three species of isolated, viable, anaerobic gut bacteria comprising: Bacteroides vulgatus, Parabacteroides distasonis, and Prevotella melaninogenica, in an amount sufficient to treat or prevent dysbiosis when administered to an individual in need thereof, and
   b. a pharmaceutically acceptable carrier.
47) A pharmaceutical composition comprising:
   a. a preparation comprising at least four species of isolated, viable, anaerobic gut bacteria selected from the group consisting of: Bacteroides fragilis, Bacteroides ovatus, Bacteroides vulgatus, Parabacteroides distasonis, and Prevotella melaninogenica, in an amount sufficient to treat or prevent a dysbiosis when administered to an individual in need thereof, and
   b. a pharmaceutically acceptable carrier.
48) A pharmaceutical composition comprising:
   a. a preparation comprising isolated, viable, anaerobic gut bacteria including each of Bacteroides fragilis, Bacteroides ovatus, Bacteroides vulgatus, Parabacteroides distasonis, and Prevotella melaninogenica, in an amount sufficient to treat or prevent dysbiosis when administered to an individual in need thereof, and
   b. a pharmaceutically acceptable carrier.
49) The pharmaceutical composition of any one of paragraphs 45-48, wherein the dysbiosis is associated with an inflammatory disease or a metabolic disease or disorder
50) The pharmaceutical compositions of any one of paragraphs 45-48, wherein the dysbiosis is associated with an atopic disease or disorder.
51) The pharmaceutical composition of any one of paragraphs 45-48, which comprises not more than forty species of viable, anaerobic gut bacteria.
52) The pharmaceutical composition of any one of paragraphs 45-48, which comprises not more than thirty species of viable, anaerobic gut bacteria.
53) The pharmaceutical composition of any one of paragraphs 45-48, which comprises not more than twenty species of viable, anaerobic gut bacteria.
54) The pharmaceutical composition of any one of paragraphs 45-48, which comprises not more than fifteen species of viable, anaerobic gut bacteria.
55) The pharmaceutical composition of any one of paragraphs 45-48, which comprises not more than eleven species of viable, anaerobic gut bacteria.
56) The pharmaceutical composition of any one of paragraphs 45-50, which further comprises at least one species of bacteria selected from the group consisting of: Clostridium ramosum, Clostridium scindens, Clostridium hiranonsis, Clostridium bifermentans, Clostridium leptum, and Clostridium sardiniensis.
57) The pharmaceutical composition of any one of paragraphs 45-56, which further comprises at least two species of bacteria selected from the group consisting of: Clostridium ramosum, Clostridium scindens, Clostridium hiranonsis, Clostridium bifermentans, Clostridium leptum, and Clostridium sardiniensis.
58) The pharmaceutical composition of any one of paragraphs 45-57, which further comprises at least three species of bacteria selected from the group consisting of: Clostridium ramosum, Clostridium scindens, Clostridium hiranonsis, Clostridium bifermentans, Clostridium leptum, and Clostridium sardiniensis.
59) The pharmaceutical composition of any one of paragraphs 45-58, which further comprises at least four species of bacteria selected from the group consisting of: Clostridium ramosum, Clostridium scindens, Clostridium hiranonsis, Clostridium bifermentans, Clostridium leptum, and Clostridium sardiniensis.
60) The pharmaceutical composition of any one of paragraphs 45-59, which further comprises at least five species of bacteria selected from the group consisting of: Clostridium ramosum, Clostridium scindens, Clostridium hiranonsis, Clostridium bifermentans, Clostridium leptum, and Clostridium sardiniensis.
61) The pharmaceutical composition of any one of paragraphs 45-60, which further comprises Clostridium ramosum, Clostridium scindens, Clostridium hiranonsis, Clostridium bifermentans, Clostridium leptum, and Clostridium sardiniensis.
62) The pharmaceutical composition of any one of paragraphs 45-61, wherein the microbial species do not comprise any of the Species Escherichia coli, Klebsiella pneumoniae, Proteus mirabilis, Enterobacter cloacae, Bilophila wadsworthia, Alistipes onderdonkii, Desulfovibrio species, Lactobacillus johnsoni, and Parasutterella excrementihominis.
63) The pharmaceutical composition of any one of paragraphs 45-62, wherein the microbial species do not comprise bacteria of the Genera Bilophila, Enterobacter, Escherichia, Klebsiella, Proteus, Alistipes, Blautia, Desulfovibrio, and Parasutterella
64) The pharmaceutical composition of any one of paragraphs 45-63, wherein the microbial species do not comprise bacteria of the Families Desulfovibrionaceae, Enterobacteriaceae, Rikenellaceae, and Sutterellaceae.
65) The pharmaceutical composition of any one of paragraphs 45-64, wherein the microbial species do not comprise bacteria of the Families Lactobacillaceae, or Enterbacteriaceae
66) The pharmaceutical composition of any one of paragraphs 45-65, wherein the microbial species do not comprise bacteria of the Order Burkholdales, Desulfovibrionales, or Enterobacteriales.
67) The pharmaceutical composition of any one of paragraphs 45-66, wherein the pharmaceutically acceptable carrier comprises an enteric coating composition that encapsulates the microbial consortium.
68) The pharmaceutical composition of any one of paragraphs 45-67, formulated to deliver the viable bacteria to the small intestine.
69) The pharmaceutical composition of any one of paragraphs 45-68, wherein the pharmaceutically acceptable carrier comprises a capsule, gel, pastille, tablet or pill.
70) The pharmaceutical composition of any one of paragraphs 45-69, wherein the consortium of viable gut bacteria is formulated with an enteric coating.
71) The pharmaceutical composition of any one of paragraphs 45-70, wherein the species of viable gut bacteria are human gut bacteria.
72) The pharmaceutical composition of any one of paragraphs 45-71, wherein the species of viable gut bacteria are present in substantially equal biomass.
73) The pharmaceutical composition of any one of paragraphs 45-72, wherein the composition is formulated to deliver a dose of at least 1×10⁹ colony forming units (CFUs).
74) The pharmaceutical composition of any of paragraphs 45-73, wherein the composition is formulated to deliver at least 1×10⁹ CFUs in less than 30 capsules per one-time dose.
75) The pharmaceutical composition of any of paragraphs 45-74, wherein the composition is frozen for storage.
76) The pharmaceutical composition of any of paragraphs 45-75, wherein the species of viable gut bacteria are encapsulated under anaerobic conditions.
77) The pharmaceutical composition of paragraph 76, wherein anaerobic conditions comprise one or more of the following:
   a. oxygen impermeable capsules,
   b. addition of a reducing agent including N-acetylcysteine, cysteine, or methylene blue to the composition, or
   c. use of spores for organisms that sporulate.
78) The pharmaceutical composition of paragraph 67, wherein the enteric coating comprises a polymer, nanoparticle, fatty acid, shellac, or a plant fiber.
79) The pharmaceutical composition of any one of paragraphs 45-78, further comprising a prebiotic composition.
80) The pharmaceutical composition of any one of paragraphs 45-79, wherein the composition is encapsulated, a lyophilisate, formulated in a food item, or is formulated as a liquid, gel, fluid-gel, or nanoparticles in a liquid.
81) A method for treating, or preventing a dysbiosis in a subject, the method comprising: administering to a subject a pharmaceutical composition of any one of paragraphs 1-80, thereby treating, or preventing dysbiosis in the subject.
82) A method for the treatment, or prevention of gut inflammation or a metabolic disease or disorder, the method comprising: administering to a subject a pharmaceutical composition of any one of paragraphs 1-80, thereby treating, or preventing the gut inflammation or metabolic disease or disorder in the subject.
83) A method for the treatment, or prevention of an atopic disease or disorder, the method comprising: administering to a subject a pharmaceutical composition of any one of paragraphs 1-80, thereby treating, or preventing the atopic disease or disorder in the subject.
84) The method of paragraph 83, wherein the atopic disease or disorder is selected from the group consisting of: food allergy, eczema, asthma, and rhinoconjunctivitis.
85) The method of any one of paragraphs 81-84, wherein the pharmaceutical composition is administered by oral administration, enema, suppository, or orogastric tube.
86) The method of any one of paragraphs 81-85, wherein the species of viable gut bacteria are isolated and/or purified from a subject known to be tolerant to a selected food allergen.
87) The method of any one of paragraphs 81-86, wherein the species of viable gut bacteria are prepared by culture under anaerobic conditions.
88) The method of any one of paragraphs 81-87, wherein the species of viable gut bacteria are formulated to maintain anaerobic conditions.
89) The method of paragraph 88, wherein anaerobic conditions are maintained by one or more of the following:
   a. oxygen impermeable capsules,
   b. addition of a reducing agent including N-acetylcysteine, cysteine, or methylene blue to the composition, or
   c. use of spores for organisms that sporulate
90) The method of any one of paragraphs 81-89, further comprising administering a prebiotic composition.
91) The method of any one of paragraphs 81-90, wherein the pharmaceutical composition is enteric coated.
92) The method of any one of paragraphs 81-91, wherein the treatment administered prevents and/or reverses Tₕ2 programming.
93) The method any one of paragraphs 81-92, wherein the subject is a human subject.
94) The method of any one of paragraphs 81-93, wherein the subject is under the age of 2 years old.
95) The method of any one of paragraphs 81-93, wherein the subject is age 2 to under 5 years old.
96) The method of any one of paragraphs 81-93, wherein the subject is age 5 to under 12 years old
97) The method of any one of paragraphs 81-93, wherein the subject is age 12 to under 18 years old.
98) The method of any one of paragraphs 81-93, wherein the subject is age 18 to under 65 years old.
99) The method of any one of paragraphs 81-93, wherein the subject is over age 65 years old.
100) The method any one of paragraphs 81-99, further comprising a step of diagnosing the subject as having or as likely to develop an inflammatory disease or an atopic disease or disorder.
101) The method any one of paragraphs 81-100, further comprising a step of testing a fecal sample from the subject for the presence and/or levels of one or more of the bacteria in the pharmaceutical composition.
102) The method of any one of paragraphs 83-101, wherein the atopic disease is a food allergy, and wherein the food allergy comprises allergy to soy, wheat, eggs, dairy, peanuts, tree nuts, shellfish, fish, mushrooms, stone fruits and/or other fruits.
103) The method any one of paragraphs 83-102, wherein the pharmaceutical composition is administered before the first exposure to a potential food allergen.
104) The method any one of paragraphs 83-103, wherein the pharmaceutical composition is administered upon clinical signs of atopic symptoms.
105) The method any one of paragraphs 81-104, wherein the pharmaceutical composition is administered to an individual diagnosed with a food allergy.
106) The method any one of paragraphs 81-105, wherein the subject is pretreated with an antibiotic.
107) A method for reducing or eliminating a subject's immune reaction to an allergen, the method comprising: administering to a subject a pharmaceutical composition of any of paragraphs 1-80, thereby reducing or eliminating a subject's immune reaction to an allergen.
108) The method of paragraph 107, wherein the allergen is a food allergen.
109) The method of paragraph 108, wherein the allergen is selected from the group consisting of: soy, wheat, eggs, dairy, peanuts, tree nuts, shellfish, fish, mushrooms, stone fruits and other fruits.
110) The method of any one of paragraphs 107-109, wherein the pharmaceutical composition is administered by oral administration, enema, suppository, or orogastric tube.
111) The method of any one of paragraphs 107-110, wherein the treatment prevents and/or reverses T_{H}2 programming.
112) The method of any one of paragraphs 107-111, wherein the subject is a human subject.
113) The method of any one of paragraphs 107-112, wherein the subject is under the age of 2 years old.
114) The method of any one of paragraphs 107-112, wherein the subject is age 2 to under 5 years old.
115) The method of any one of paragraphs 107-112, wherein the subject is age 5 to under 12 years old
116) The method of any one of paragraphs 107-112, wherein the subject is age 12 to under 18 years old.
117) The method of any one of paragraphs 107-112, wherein the subject is age 18 to under 65 years old.
118) The method of any one of paragraphs 107-112, wherein the subject is over age 55 years old.
119) The method of any one of paragraphs 107-118, further comprising a step of diagnosing the subject as having an IgE-mediated allergy.
120) The method of any one of paragraphs 107-119, further comprising a step of testing a fecal sample from the subject for the presence and/or levels of one or more of the bacteria in the pharmaceutical composition.
121) The method of paragraph 119, wherein the IgE-mediated allergy is a food allergy selected from the group consisting of: allergy to soy, wheat, eggs, dairy, peanuts, tree nuts, shellfish, fish, mushrooms, stone fruits and other fruits.
122) The method of any one of paragraphs 107-121, wherein the pharmaceutical composition is administered after an initial exposure and/or reaction to a potential allergen,
123) The method of any one of paragraphs 107-122, wherein the biomass of each of the microbes in the administered compositions is greater than the biomass of each of the microbes relative to a reference.
124) The method of any one of paragraphs 107-123, wherein the subject is pretreated with an antibiotic.
125) The method of any one of paragraphs 107-124, wherein the subject is pretreated with a fasting period not longer than 24 hours.
126) A method of monitoring a subject's microbiome, the method comprising: determining the presence and/or biomass in a biological sample obtained from a subject, and wherein if at least two or more species selected from the group consisting of Bacteroides fragilis, Bacteroides ovatus, Bacteroides vulgatus, Parabacteroides distasonis, and Prevotella melaninogenica, are absent or low relative to a reference, the subject is treated with the pharmaceutical composition of any one of paragraphs 1-80.
127) The method of paragraph 126, wherein the method further comprises predicting that a subject will have an immune response to an allergen when the at least two members are absent, the biomass of the at least two members is low relative to a reference, or at least one member of a dysbiotic species is present or elevated relative to a reference.
128) The method of paragraph 127, wherein the method is repeated at least one additional time.
129) The method of paragraph 126, wherein the biological sample is a fecal sample.
130)A method of treating atopic disease or disorder in an individual in need thereof, the method comprising administering a composition of any one of paragraphs 1-80 to the individual.
131) The method of paragraph 130, wherein the administration shifts the balance of Tₕ1/Tₕ2 cells towards Tₕ1 T cells.
132) The method of paragraph 130, wherein the administration reduces the number or activity of Tₕ2 T cells.
133) A method of reducing the number or activity of Tₕ2 cells in a tissue of an individual in need thereof, the method comprising administering a pharmaceutical composition of any one of paragraphs 1-80 to the individual.
134) The method of paragraph 133, wherein the tissue is a gut tissue.

### Examples

The data provided herein, e.g., in the figures and elsewhere, show that a microbial consortium of several species (i.e., 3, 4, 5, or 6 species, for example) can protect against developing dysbiosis and associated conditions, an atopic disease, or food allergy in a mouse model. Treatment with such a consortium of bacteria can reverse Tₕ2 programming of T_{regs}. Treatment and/or prevention of dysbiosis and associated conditions, an atopic disease, or food allergy using a similar microbial consortium of microbes in humans is specifically indicated.

Further methods for testing or measuring the efficacy of a microbial consortium in a mouse model of food allergy are known in the art and/or can be found in e.g., Noval Rivas et al. J Allergy Clin Immunol (2013) 131(1) 201-212 or Noval Rivas et al. Immunity (2015) 42:512-523, the contents of which are each incorporated herein in their entirety.

### Example 1: Therapeutic Microbiota to Treat Food Allergy

### Summary

Food allergy is a growing national problem, affecting 6% of children, and 3% of US teens and adults. Unfortunately for these children and their families, the standard of care remains to avoid offending foods and manage symptoms as they occur. Therapies using oral desensitization, alone or with anti-IgE (Omalizumab™), remain experimental with limited success. Needed are therapies that target the aberrant immune responses. As such, this study shows the use of gut microbiota as a therapeutic intervention to promote tolerizing responses that can prevent or mitigate effects of Tₕ2/allergic responses.

Food allergies occur with development of Tₕ2-allergic responses to foodstuffs, in contrast to tolerizing T-regulatory responses that mitigate such responses mucosally. The Tₕ2 responses promote food antigen-specific IgE antibody and recruitment of mucosal mast cells, in contrast to regulatory responses, which inhibit these effects. Once sensitized to one or more food antigens, re-exposure can induce life-threatening anaphylactic responses. Capacity to promote tolerizing responses supports a broad-based therapeutic approach that can act at the earliest stages of exposure as well as in the already-sensitized patient to prevent aberrant allergic responses across a spectrum of foodstuffs.

Leveraging the genetically susceptible IL4RA F709 mouse model of food allergy defined human commensal communities that can both prevent and cure food allergies in preclinical models have been developed. These communities leverage a new therapeutic pathway for patients—immunomodulation from the luminal side of the gut, the space in which the gut microbiota resides. Human gut microbiota consists of many hundreds of species that provide critical functions in normal human development and health, from maturing of the immune system, providing essential nutrients such as B vitamins and vitamin K, and assisting in digestion and metabolism of dietary and exogenous compounds, including drugs and ingested foodstuffs.

### Consortia Development

For pre-clinical studies in mice the component members are grown individually in nutrient-rich media under appropriate anaerobic conditions, quantitated for biomass, and then the consortium is mixed under anaerobic conditions with approximately equal biomass of each component organism to a final concentration ˜5.0×10⁸ colony forming units (CFU)/mL. Input culture volumes for each species have been in the range of 100 mL-1 L As needed, cultures with a stationary phase biomass <5×10⁸ CFU/mL are concentrated by centrifugation with re-suspension handled under anaerobic conditions.

When mixed, the total biomass remains approximately 5×10⁸ CFU/mL. 2 mL aliquots are placed in cryovials with an anaerobic/pre-reduced atmosphere, snap frozen on liquid nitrogen and stored at −80° C. until use. Rapid freezing has shown to have <½ log effects on the biomass of the component organisms and no effect on efficacy in animal models. For studies, tubes are thawed and mice administered 200 uL of this solution weekly to twice weekly by oral gavage, resulting in a total introduced biomass of 1×10⁸ CFU/mouse. The measurements of gut contents in adult mice (stomach through anus) range from 4-8 mL of material. The gavaged consortium is thus 2.5-5% of the total volume of contents in the mouse gut and >10% of the volume of contents in the small bowel.

In terms of pre-existing microbial biomass from the conventional microbiota—the mouse small intestine on average has ˜10⁴ CFU/mL in proximal duodenum with increase to 10⁸ CFU/mL in the ileum. Biomass increases to 10⁹-10¹⁰ CFU/mL in the cecum and colon.

From the standpoint of microbial biomass at locations in the mouse small bowel, the primary site of action of the consortia to promote regulatory T cell responses, the consortium is 10,000× the biomass of the duodenal microbiota, and 1-2× the biomass of the jejunal and ileal microbiota.

In comparison, the adult human gut may contain 4.5 L of material, of which 1 L relates to ingested foodstuffs with 3.5 L of secretions including saliva, bile, and other fluids from the pancreas and intestines. These fluids and electrolytes are largely resorbed in the right colon, subsequent to fecal compaction and passage. Within the intestines, the biomass of organisms also varies, with the highest concentration in the cecum and right side of the colon (10¹⁰-10¹² CFU/mL). In contrast, in the small intestine—the believed site of action, the biomass also ranges from 10⁴ CFU/mL in the duodenum to 10⁸ CFU/mL in the ileum.

### Human Dosing

The CFU/dose for humans is based on the following parameters:

(1) Treatment of Clostridium difficile with oral capsule formulations of human stool: Data from OpenBiome and other groups have shown successful treatment of Clostridium difficile colitis with capsule formulation that administers 3-5×10⁹ CFU in a range of 12-30 capsules taken per one-time dose. A standard 12-capsule regimen is expected to deliver approximately 4.2×10⁹ CFU per dose.

(2) Alter the small intestinal microbiota to promote immunomodulation. An encapsulated formulation releasing contents in the proximal small bowel will deliver a dose of 3-5×10⁹ CFU, exceeding the duodenal biomass by a factor of 10,000, and approaching a 1:1 ratio with communities in the jejunum and ileum.

Other formulations including nanoparticles in liquid, with optional pre-biotic compounds to enhance colonization and viability, or a reconstituted lyophilisate are contemplated, however given the need to prevent exposure to oxygen and for ease of storage and administration, the first formulation uses encapsulated material.

### Administration

Given the obligatory anaerobic nature of the component species, phase I studies use encapsulated formulations with the following properties:

Stage I:
Can be swallowed by an adult or child >8 years of age.
Excludes oxygen
Holds a volume so that a person needs to take 15 or less capsules per dose
Can be stored frozen (−20° C. or −80° C.) and thawed prior to administration
Releases contents after passage through the stomach

In one embodiment, the capsules used by OpenBiome™ for oral FMT therapy are used to encapsulate the GP-IIa mixtures. Other options are also available commercially and contemplated herein. In some embodiments, the capsules consist of frozen material (in order to ensure an adequate product) that is thawed prior to administration and is encapsulated, free of oxygen, with material that survives intact into the small intestine.

### Scale of Culture

The animal studies used pilot cultures in the range of 100-1000 mL. To generate human doses, the culture is scaled by at least a factor of 10 The following steps are contemplated herein.

(1) Perform growth curves in different media conditions—to optimize growth conditions and correlate an OD600 with plated biomass.
(2) Grow the component members anaerobically in liquid media. Media is pre-reduced and incubated at 37° C. with some level of agitation (e.g., 150 rpm or with stirring/fermenter baffles) to insure a maximal culture density. Depending upon the fermenter system, nitrogen or anaerobic gas mixtures can be spared to maintain anaerobic conditions. However, none of the component species require H₂ or CO₂ for growth, beyond maintaining appropriate acid/base balance.
(3) Concentrate select members as needed to obtain desired input density: commonly done by centrifugation at 5-1 OK RPM with pull-off of supernatant under anaerobic conditions and resuspension in a lesser volume of new culture media or appropriate suspension buffer. The new culture density is confirmed by OD600 reading and viability by plating to solid media.
(4) Aggregate the cultured into the combined consortium: Estimated biomass from the OD600 readings are used to estimate the volume and prepare the aggregate.
(5) Prepare capsules: done under anaerobic conditions to preserve viability.
(6) Store capsules: optimal to store at conditions available clinically, e.g. −20° C.
(7) Quality Control: In addition to QC for prior steps and media, the final community will be evaluated to insure the appropriate species are present and in desired viable biomass. Analyses on materials for pre-clinical studies used 16S rRNA gene phylotyping with culture a qPCR-based methods. Metagenomic approaches may also be used to rule-out contamination with nonbacterial species or viruses.

It is further contemplated herein that growth conditions are optimized for the scaled cultures.

In some embodiments, media formulations are developed such that they lack animal products and/or substrates that might be associated with sensitizing antigens in foodstuffs. In addition, one of skill in the art can assess if additives to the inoculum enhance viability in capsules and once released in vivo. Materials can include preservatives and prebiotic compounds.

Stage II: It is further contemplated herein that the consortia described herein are formulated as a liquid formulation that can be administered to infants and young children. It is contemplated herein that such formulations comprise mixtures of spores from sporulating species, leveraging non-sporulating obligate anaerobes with limited aerotolerance, and including reducing factors in a liquid formula to buffer against short-term exposure to oxygen in ambient air and upon entry into the digestive tract. Compounds such as the amino acid cysteine or n-acetylcysteine, which have been used therapeutically in infants and have a robust safety profile are contemplated.

Additional pre-clinical animal models for use in testing formulations include e.g., neonatal swine models of food allergy, including ones for foodstuffs common in the diet of both humans and pigs.

### Example 2: OTU Clustering Method for Data from Human and Animal Studies

DNA Extraction and Sequencing for 16S rRNA Gene Phylotyping. A Multiplexed amplicon library covering the V4 region of the 16S rDNA gene was generated from DNA extracted from human stool, mouse fecal pellets or segments of snap-frozen gut tissues using MO BIO™ Power-Fecal™ DNA Isolation Kits (MO BIO™ Laboratories) with custom modification to enhance lysis of Gram positive commensals with thick cell walls. The rest of library preparation followed the protocol of with dual-index barcodes. Aggregated libraries are sequenced with paired-end 250 bp reads on the Illumina™ MiSeq platform. The aggregate library pool was size selected from 300-500 bp on a Pippin™ prep 1.5% agarose cassette (Sage Sciences™) according to the manufacturer's instructions. Concentration of the pool is measured by qPCR (Kapa Biosystems™) and loaded onto the MiSeq™ (Illumina™) at 6-9 pM with 20% phiX spike-in to compensate for low base diversity according to Illumina™'s standard loading protocol.

16S rRNA Data preprocessing. Sequencing aims to obtain 10-50K usable reads per sample after quality filtering. Raw sequencing reads were processed using the mothur software package (v. 1.35.1) and custom Python and R scripts, which perform de-noising, quality filtering, alignment against the ARB Silva reference database of 16S rDNA gene sequences, and clustering into Operational Taxonomic Units (OTUs) at 97% identity.

16S rRNA Data Analysis. To statistically test for differences between control and food allergic subjects in abundances of microbial taxa (OTUs), the DESeq2 software package was employed to support of analyses relative to host co-variates such as age, food allergy status, diet and antibiotic use in human cohort, OTUs showing significant differences were defined by: (1) adjusted p-value <=0.1; (2) relative abundance >=0.01 in either control or food allergic groups; (3) absolute value of log 2 fold changes >=2.

To improve the resolution of taxonomic calls and show phylogenetic relationships, a separate method used the Pplacer software package to perform phylogenetic placement of individual OTU. Pplacer uses a likelihood-based methodology to place short sequencing reads of 16S rRNA amplicons on a reference tree, and also generates taxonomic classifications of the short sequencing reads using a least common ancestor-based algorithm. The reference tree required for phylogenetic placement is generated using full-length or near full-length (>1,200 nt) 16S rDNA sequences of type species from the Ribosomal Database Project (RDP).

For all statistical testing for 16S rDNA data analysis, p-values were adjusted for multiple hypothesis testing using the method of Benjamini and Hochberg (BH). Heat map plots are generated using custom R scripts.

Alpha diversity values (richness of a sample in terms of the diversity of the OTUs observed in it) were calculated using Shannon entropy to measure diversity in each sample. Beta-diversity values (distance between samples based on differences in OTUs present in each sample) were calculated using the unweighted/weighted Unifrac dissimilarity measure, to assess differences in overall microbial community structure.

(3) OTU Mappings of the Defined Species

The following operational taxonomic units map to the defined species, as identified in gnotobiotic mice colonized with these consortia (TABLE 2 and TABLE 3). Fecal pellets were subjected to the above described 16S rRNA gene phylotyping over the V4 variable region.

TABLE 2
Mapping of the defined therapeutic species to OTU based on the 16S rRNA
V4 region.

| Species | OTU taxonomic mappings, V4 region- |
|---|---|
| Bacteroides | Bacteria:Bacteroidetes:Bacteroidia:Bacteroidales |
| fragilis | Bacteria:Bacteroidetes:Bacteroidia:Bacteroidales:Bacteroidaceae |
| | Bacteria:Bacteroidetes:Bacteroidia:Bacteroidales:Bacteroidaceae:Bacteroides |
| Bacteroides | Bacteria:Bacteroidetes:Bacteroidia:Bacteroidales |
| thetaiotaomicron | Bacteria:Bacteroidetes:Bacteroidia:Bacteroidales:Bacteroidaceae |
| | Bacteria:Bacteroidetes:Bacteroidia:Bacteroidales:Bacteroidaceae:Bacteroides |
| Bacteroides | Bacteria:Bacteroidetes:Bacteroidia:Bacteroidales |
| ovatus | Bacteria:Bacteroidetes:Bacteroidia:Bacteroidales:Bacteroidaceae |
| | Bacteria:Bacteroidetes:Bacteroidia:Bacteroidales:Bacteroidaceae:Bacteroides |
| Clostridium | Bacteria:Firmicutes:Clostridia:Clostridiales:Peptostreptococcaceae |
| bifermentans | Bacteria:Firmicutes:Clostridia:Clostridiales:Peptostreptococcaceae:Clostridium |
| | cluster XI |
| Clostridium | Bacteria:Firmicutes:Clostridia:Clostridiales:Peptostreptococcaceae |
| hiranonsis | Bacteria:Firmicutes:Clostridia:Clostridiales:Peptostreptococcaceae:Clostridium |
| | cluster XI |
| Clostridium | Bacteria:Firmicutes:Clostridia:Clostridiales:Ruminococcaceae |
| leptum | Bacteria:Firmicutes:Clostridia:Clostridiales:Ruminococcaceae:Clostridium |
| | cluster IV |
| Clostridium | Bacteria:Firmicutes:Erysipelotrichia:Erysipelotrichales:Erysipelotrichaceae |
| ramosum | Erysipelotrichales:Erysipelotrichaceae:Clostridium cluster XVIII |
| Clostridium | Bacteria:Firmicutes:Clostridia:Clostridiales: |
| sardiniensis | Bacteria:Firmicutes:Clostridia:Clostridiales:Clostridiaceae I |
| (absonum) | |
| Clostridium | Bacteria:Firmicutes:Clostridia:Clostridiales:Lachnospiraceae |
| scindens | Bacteria:Firmicutes:Clostridia:Clostridiales:Lachnospiraceae:Clostridium |
| | cluster XIVa |
| Parabacteroides | Bacteria:Bacteroidetes:Bacteroidia:Bacteroidales:Porphyoromondaceae |
| goldsteinii | Bacteria:Bacteroidetes:Bacteroidia:Bacteroidales:Porphyoromondaceae: |
| | Parabacteroides |
| Prevotella | Bacteria:Bacteroidetes:Bacteroidia:Bacteroidales: |
| tannerae | Bacteria:Bacteroidetes:Bacteroidia:Bacteroidales:Prevotellaceae |
| | Bacteria:Bacteroidetes:Bacteroidia:Bacteroidales:Prevotellaceae: |
| | Prevotella |

TABLE 3
Mapping of the dysbiotic consortium species to OTU based on the 16S rRNA
V4 region

| Species | OTU mapping |
|---|---|
| Bilophila | Bacteria:Proteobacteria:Deltaproteobacteria:Desulfovibrionales |
| wadsworthia | Bacteria:Proteobacteria:Deltaproteobacteria:Desulfovibrionales:Desolfovibrionaceae |
| | Bacteria:Proteobacteria:Deltaproteobacteria:Desulfovibrionales: |
| | Desolfovibrionaceae:Bilophila |
| Enterobacter | Bacteria:Proteobacteria:Gammaproteobacteria:Enterobacteriales |
| cloacae | Bacteria:Proteobacteria:Gammaproteobacteria:Enterobacteriales:Enterobacteriaceae |
| | Bacteria:Proteobacteria:Gammaproteobacteria:Enterobacteriales:Enterobacteriaceae: |
| | Enterbacter |
| Escherichia | Bacteria:Proteobacteria:Gammaproteobacteria:Enterobacteriales |
| coli | Bacteria:Proteobacteria:Gammaproteobacteria:Enterobacteriales:Enterobacteriaceae |
| | Bacteria:Proteobacteria:Gammaproteobacteria:Enterobacteriales:Enterobacteriaceae: |
| | Escherichia |
| Klebsiella | Bacteria:Proteobacteria:Gammaproteobacteria:Enterobacteriales |
| pneumonia | Bacteria:Proteobacteria:Gammaproteobacteria:Enterobacteriales:Enterobacteriaceae |
| | Bacteria:Proteobacteria:Gammaproteobacteria:Enterobacteriales:Enterobacteriaceae: |
| | Klebsiella |
| Proteus | Bacteria:Proteobacteria:Gammaproteobacteria:Enterobacteriales |
| mirabilis | Bacteria:Proteobacteria:Gammaproteobacteria:Enterobacteriales:Enterobacteriaceae |
| | Bacteria:Proteobacteria:Gammaproteobacteria:Enterobacteriales:Enterobacteriaceae: |
| | Proteus |

Species of microorganisms associated with protection from the development of food allergy were identified in a longitudinal study of pediatric human subjects (TABLE 4).

TABLE 4
Additional “beneficial” OTU identified in the longitudinal pediatric human
cohort as associated with protection from development of food allergy.

| | Nearest species |
|---|---|
| | mapping(s) with |
| OTU taxonomic mappings with sequencing of the V4 region- | pplacer |
| Bacteria:Firmicutes:Clostridia:Clostridiales:Lachnospiraceae | Clostridium |
| Bacteria:Firmicutes:Clostridia:Clostridiales:Lachnospiraceae:Clostridium | hathewayi |
| cluster XIVa | |
| Bacteria:Firmicutes:Clostridia:Clostridiales:Lachnospiraceae:Hungatella | |
| Bacteria:Firmicutes:Clostridia:Clostridiales:Lachnospiraceae | Clostridium nexile, |
| Bacteria:Firmicutes:Clostridia:Clostridiales:Lachnospiraceae:Clostridium | Clostridium |
| cluster XIVa | hylemonae, |
| | Clostridium |
| | glycyrrhizindyticum, |
| | Clostridium |
| | scindens, |
| | Clostridium |
| | lavalense, |
| | Clostridium |
| | fimetarium, |
| | Clostridium |
| | symbiosum |
| Bacteria:Firmicutes:Clostridia:Clostridiales:Ruminococcaceae | Clostridium |
| Bacteria:Firmicutes:Clostridia:Clostridiales:Ruminococcaceae:Clostridium | sporosphaeroides |
| cluster IV | |
| Bacteria:Firmicutes:Negativicutes:Selenomonadales:Veillonellaceae | Dialister |
| Bacteria:Firmicutes:Negativicutes:Selenomonadales:Veillonellaceae:Dialister | proprionicifaciens, |
| | Dialister |
| | succinatiphilus |
| Bacteria:Bacteroidetes:Bacteroidia:Bacteroidales:Porphyoromondaceae | Parabacteroides |
| Bacteria:Bacteroidetes:Bacteroidia:Bacteroidales:Porphyoromondaceae: | distasonis, |
| Parabacteroides | Parabacteroides |
| | goldsteinii, |
| | Parabacteroides |
| | merdae |
| Bacteria:Firmicutes:Clostridia:Clostridiales:Peptostreptococcaceae | Peptostreptococcus |
| Bacteria:Firmicutes:Clostridia:Clostridiales:Peptostreptococcaceae: | anaerobius |
| Peptostreptococcus | |
| Bacteria:Firmicutes:Clostridia:Clostridiales:Ruminococcaceae | Subdoligranulum |
| Bacteria:Firmicutes:Clostridia:Clostridiales:Ruminococcaceae: | variabile |
| Subdoligranulum | |
| Bacteria:Firmicutes:Negativicutes:Selenomonadales:Veillonellaceae | Veilonella ratti |
| Bacteria:Firmicutes:Negativicutes:Selenomonadales:Veillonellaceae:Veillonella | |

Microorganisms that are associated with the development of food allergy were identified in a longitudinal study of pediatric human subjects (TABLE 5).

TABLE 5
Additional “dysbiotic” OTU identified in the longitudinal pediatric human
cohort as associated with development of food allergy.

| | Nearest species |
|---|---|
| | mapping |
| OTU taxonomic mappings with sequencing of the V4 region | with pplacer |
| Bacteroidetes:Bacteroidia:Bacteroidales:Rikenellaceae: | Alistipes |
| Bacteroidetes:Bacteroidia:Bacteroidales:Rikenellaceae:Alistipes | onderdonkii |
| Firmicutes:Clostridia:Clostridiales:Lachnospiraceae | Blautia |
| Firmicutes:Clostridia:Clostridiales:Lachnospiraceae:Blautia | wexlerae, |
| | Blautia |
| | henselae |
| Bacteria:Proteobacteria:Deltaproteobacteria:Desulfovibrionales | Bilophila |
| Bacteria:Proteobacteria:Deltaproteobacteria:Desulfovibrionales: | wadsworthia, |
| Desolfovibrionaceae | Desulfovibrio |
| Bacteria:Proteobacteria:Deltaproteobacteria:Desulfovibrionales: | species |
| Desolfovibrionaceae:Bilophila | |
| Bacteria:Proteobacteria:Deltaproteobacteria:Desulfovibrionales: | |
| Desolfovibrionaceae:Bilophila:Desulfovibrio | |
| Firmicutes:Bacilli:Lactobacillales:Lactobacillaceae:Lactobacillus | Lactobacillus |
| | johnsoni |
| Bacteria:Proteobacteria:Betaproteobacteria:Burkholderales: | Parasutterella |
| Bacteria:Proteobacteria:Betaproteobacteria:Burkholderales:Sutterellaceae | excrementihominis |
| Bacteria:Proteobacteria:Betaproteobacteria:Burkholderales:Sutterellaceae: | |
| Parasutterella | |
| Firmicutes:Clostridia:Clostridiales:Lachnospiraceae | Roseburia |
| Firmicutes:Clostridia:Clostridiales:Lachnospiraceae:Roseburia | inulivorans |

### Example 3: Microbiology-Level Activities Used in Selection of Defined Species

The species in the defined consortia were selected per known biochemical, immunologic and microbiologic functions with capacity to affect beneficial immunomodulatory responses in the host. Without wishing to be bound by theory, microbiologic mechanisms of action can include the following.

### Adjuvant Effects of Microbial Products:

Described herein are embodiments of microbial products to stimulate the development, proliferation, and activity of regulatory T cells (T_{regs}) and other immune cell pathways. The production of key microbial antigens from commensal anaerobes, including their lipoteichoic acid (LTA), exo-polysaccharides (PSA), LPS, bacterial flagellin, and bacterial DNA can act through stimulating toll-like-receptor pathways (e.g., TLR→MyD88 and other immune cell pathways) to skew mucosal T cells to a regulatory vs. allergic phenotype. In contrast, published data have shown that bacterial cell wall fractions from members of the negative control consortium can promote aberrant stimulation of both allergic (Tₕ2) and pro-inflammatory (Tₕ1) responses. The distinct portions of these molecules that skew towards tolerance vs. allergy or inflammation highlight the interplay between mammalian hosts and colonizing microbiota, including the microbial products that signal the host to maintain a healthy homeostasis versus elicit pathogenic immune responses.

### Mucosal and Immunoprotective Functions of Microbial End-Products of Metabolism:

Short chain fatty acids (SCFA) are natural end-products of microbial anaerobic fermentation as are additional small molecule metabolites from anaerobic fermentation of different carbon sources. End-products such as butyrate have been shown to provide a primary energy source to the gut epithelium and to contribute to the development of tolerizing responses in mucosal locations. The consortia selected produce a dominance of butyrate and propionate from the fermentation of simple and complex carbohydrates that may be in the gut lumen, per the diet and secretion of host factors. These factors would likely act in combination with other microbial activities to mediate the desired immunomodulatory effects.

### Biochemical Activities:

The species selected perform the full complement of bile acid transformations and also transform a variety of other molecules including other cholesterol-derivatives, biogenic amines, lipids and production of aryl hydrocarbons which may serve as microbial siderophores, quorum sensing molecules and other metabolic intermediates within the microbial cell. Such metabolites are potentially capable of stimulating host aryl-hydrocarbon receptor (AHR) pathways which have also been demonstrated to promote tolerizing responses in the gut mucosa.

### Gut Conditioning:

Microbiologically, select members of the consortia are known to aid the subsequent colonization, biochemical and further immunoprotective roles of other species. Both Bacteroides fragilis and Bacteroides thetaiotaomicron, when included in defined flora, assist the growth of more fastidious members of the Bacteroidetes, Firmicutes and Actinobacteria. Clostridium ramosum has demonstrated comparable effects in defined colonizations of germ-free mice with other commensals. Effects are multi-factorial, and include maturing of gut epithelial responses, altered host secretion of glycoconjugates which can serve as carbon sources for the commensal flora, enhancing gut peristalsis and digestion, reducing lumen gut oxygen tension so more obligately anaerobic species can flourish, and releasing metabolites, and/or extracellular products of microbial digestion which support the growth of additional species by providing carbon and/or nitrogen sources, vitamins, and other essential micronutrients.

### Reducing the Biomass of Dysbiotic or Pathogenic Species:

Animal models conducted by our group have also shown that the species in the gut protect communities can reduce the biomass of the Proteobacteria species in the negative control consortium. Without wishing to be bound by theory, mechanistically these biomass reductions also reduce the antigen burden of products from these species that preferentially skew towards allergic responses.

### Example 4: Gut-Protect (GP-II) Consoritum—GPIIa

Composition of GP-IIa Bacteroides fragilis, Bacteroides ovatus, Bacteroides vulgatus, Parabacteroides distasonis, Prevotella melaninogenica

GP-IIa can be used to prevent, treat, and cure food allergy in a mouse IL4raF709 food allergy model.

### Introduction

The role of pathogenic dysbiosis in food allergy (FA) remains unclear. It was observed that FA infants exhibited dysbiotic fecal microbiota that evolved compositionally over time. Both infants and mice with FA had decreased secretory IgA and increased IgE binding to fecal bacteria, indicative of a broader breakdown of oral tolerance in FA than hitherto appreciated. A consortium of commensal human Clostridiales species, reflective of taxa impacted by dysbiosis, suppressed FA in mice and normalized the gut mucosal immune responses, as did a separate immunomodulatory consortium of human origin Bacteroidales species. The two consortia induced distinct subsets of regulatory T (T_{reg}) cells that were deficient in FA subjects and mice. Thus, different commensals act to stimulate specific T_{reg} cell populations to protect against FA, while dysbiosis impairs this regulatory response to promote disease.

Food allergy (FA) is a major public health concern, whose prevalence has grown dramatically over the past decade. FA now affects 6% of children under 5 years, and 3% of teens and adults. Most FA is acquired in the first or second year of life, indicating that early childhood exposures have profound long-term health consequences. The hygiene hypothesis stipulates that microbial exposures play a critical role in the development of protection against allergic diseases, and that alterations in those exposures, including changes in the host microbial flora, may underlie the rise in allergic diseases. In that regard, several studies have shown that factors impacting gut microbial colonization and composition early in life, including method of delivery (i.e., cesarean section), antibiotic use, and breastfeeding influence the development of atopic disease. Less information is available on the role of gut microbiota in human FA. Reduced gut microbiota diversity and an elevated ratio of the abundance of Enterobacteriaceae to Bacteroidaceae species in early infancy have been associated with subsequent food sensitization, suggesting that the initial stages of gut colonization with particular microbial communities may contribute to the development of atopic disease, including FA.

Prior studies have shown that the presence and composition of the gut microbiota influences the host's susceptibility to FA. Mice raised in a sterile environment cannot be tolerized to antigens given orally, have reduced IgA levels and IL-10 producing regulatory T (T_{reg}) cells. In contrast, colonization with Segmented Filamentous Bacteria (SFB) and Clostridia species promotes the development of EL-17 producing T cells and T_{reg} cells, respectively^{11,12}.

The results provided herein show that in a FA-prone genetic mouse model (Il4ra^{F709} mice), the acquisition of FA is associated with a gut microbiota signature that is distinct from that of FA-tolerant mice. Furthermore, transfer of fecal microbiota from FA but not tolerant mice to germ-free (GF) recipients transmitted susceptibility to FA. More recently, Stefka et al. found that sensitization to a food allergen was increased in mice that have been treated with antibiotics or were devoid of commensal microbiota. By selectively colonizing gnotobiotic mice, allergy-protective capacity was conferred by a Clostridia-predominant microbiota.

These findings suggest that unfavorable alterations in the development of the gut microbiota early in life favor the emergence of dysbiotic communities with concomitant reductions in beneficial species. In combination, such changes may result in a failure to promote tolerant immune responses, thus raising the host's susceptibility to allergic and inflammatory responses. Mechanisms by which the commensal microbiota may promote oral tolerance to food allergens include their enhancement of epithelial cell barrier integrity and elicitation of protective mucosal T_{reg} cell responses. The production of short-chain fatty acids, such as acetate, propionate and butyrate, by commensals such as Clostridia species, reinforce mucosal tolerance by recruiting and stabilizing T_{reg} cells in the gut. Colonization with commensal bacteria also expands populations of induced T_{reg}(iT_{reg}) cells in the gut.

Here, it is demonstrated that FA infants manifest an evolving dysbiosis that impacts beneficial gut commensals. Furthermore, administration of defined bacterial consortia of human-origin commensals, one composed of culturable species from the order Clostridialis and the other of species from order Bacteroidales, successfully prevented FA and suppressed established disease in FA-prone Il4ra^{F709} mice. Both consortia conferred protection by inducing protective T_{reg} cell populations, which are deficient in FA subjects and FA-prone mice. Thus, these results identify a common mechanism by which commensals prevent FA, and they underscore the potential for employing defined microbial consortia as oral microbial therapy in promoting disease prevention and remission.

### Results:

Promotion of Oral Tolerance in FA by Immunomodulatory Human Bacteroidales Species.

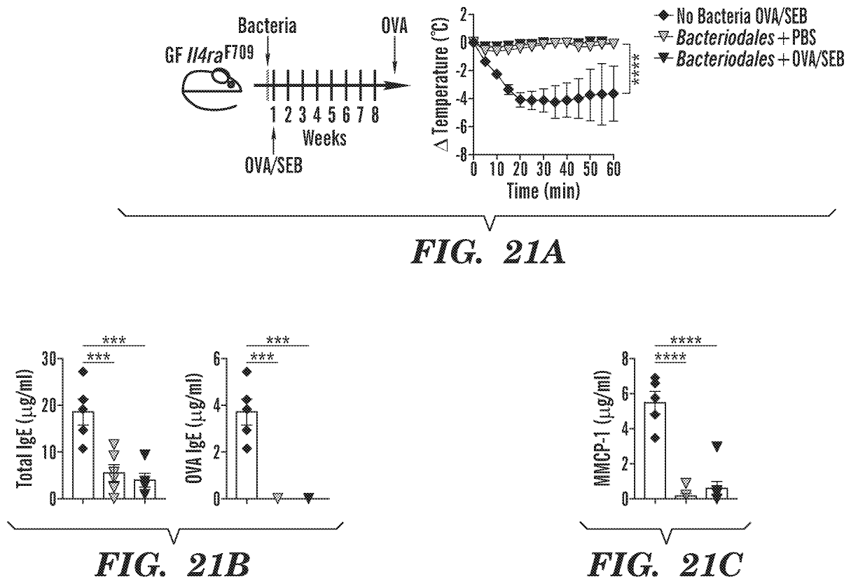

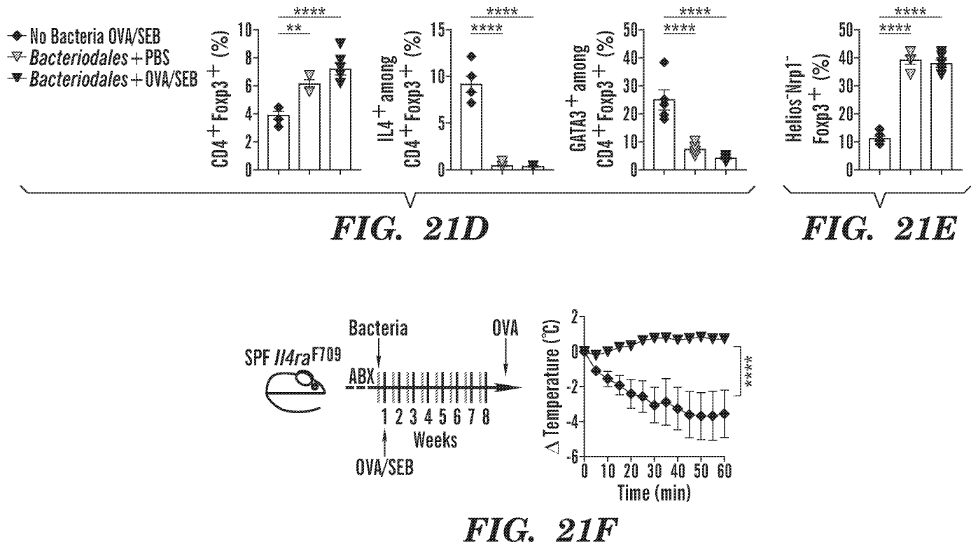

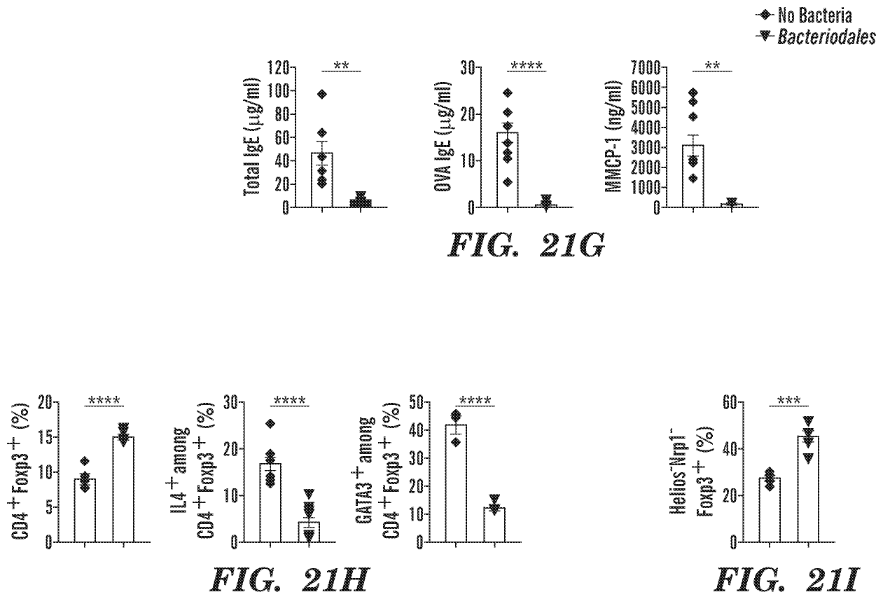

To determine if the capacity to promote oral tolerance in FA was restricted to Clostridiales species or was shared by other immunomodulatory bacteria, a consortium of five human-origin Bacteroidales species were tested, including B. fragilis, B. ovatus, B. vulgatus, P. melaninogenica, and P. distasonis (OTU24, CRS P. distasonis). Similar to the case of the Clostridiales consortium, the choice of these species reflected their availability as type species, their well-characterized genomic and metabolic profiles, ease of culturability and their previous implication in promoting T_{reg} cells in the gut. Results revealed that colonization with the Bacteroidales consortium completely protected against the induction of FA in GF Il4a^{F709} mice upon their sensitization with OVA/SEB (FIG. 21A-21E). Furthermore, the Bacteroidales consortium protected conventional SPF Il4ra^{F709} mice from developing FA when it was given in tandem with OVA/SEB during the sensitization protocol, as per the Clostridiales mix (FIG. 21F-21I). These results established that protection against FA is not a unique attribute of Clostridia species but could be affected by other immunomodulatory bacteria.

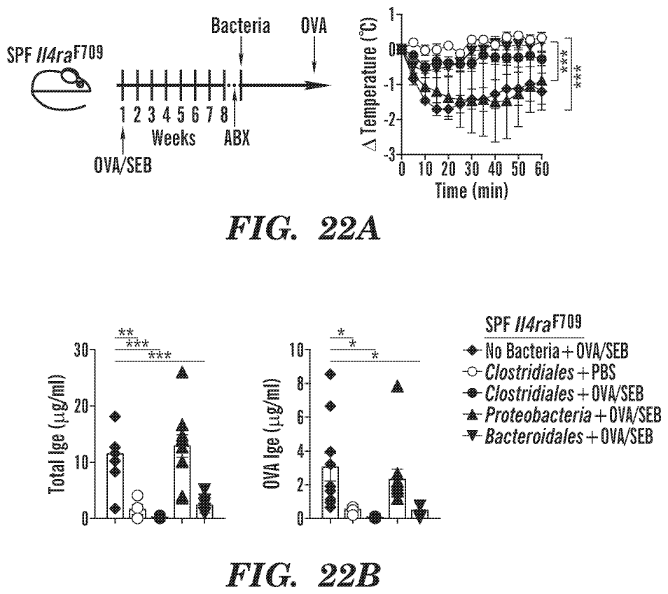

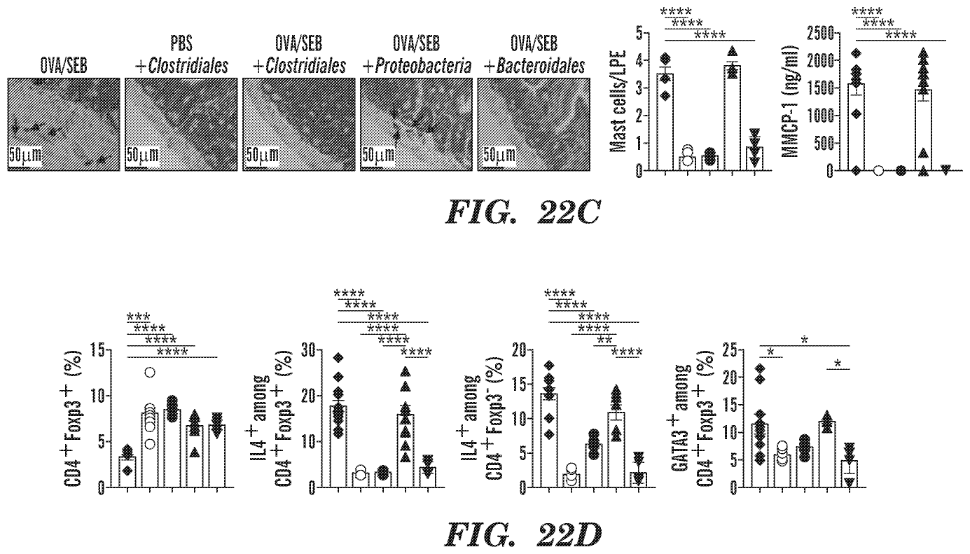

To determine whether bacteriotherapy with the Clostridiales and Bacteroidales consortia could suppress FA once the disease was established, conventional Il4raF709 mice were sensitized with OVA/SEB once weekly for eight weeks to establish disease. The mice were then treated with a short course of antibiotics and further sensitized with OVA/SEB for an additional 4 weeks with or without bacterial therapy with either the Clostridiales, Bacteroidales or Proteobacteria consortium. The mice were then challenged orally with OVA and analyzed. Results showed that therapy with either the Clostridiales or Bacteroidales but not the Proteobacteria consortium prevented the OVA/SEB-sensitized Il4ra^{F709} mice from reacting to the OVA challenge (FIG. 22A). The Clostridiales and Bacteroidales but not the Proteobacteria consortium suppressed the total and OVA-specific serum IgE responses, the rise in serum MMCP-1 post OVA challenge, and the mast cell expansion (FIG. 22B, FIG. 22C). While all bacterial consortia increased the frequencies of MLN T_{reg} cells in this disease curative model (FIG. 22D), only the Clostridiales and Bacteroidales consortia but not the Proteobacteria consortium suppressed the food allergy-associated T_{reg} cell Tₕ2 cell-like reprogramming (FIG. 22D).

Bacteroidales Consortium Demonstrates Extended Persistence In Vivo in Mice after a Single Dose.

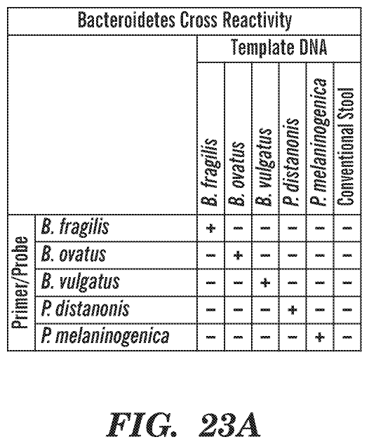

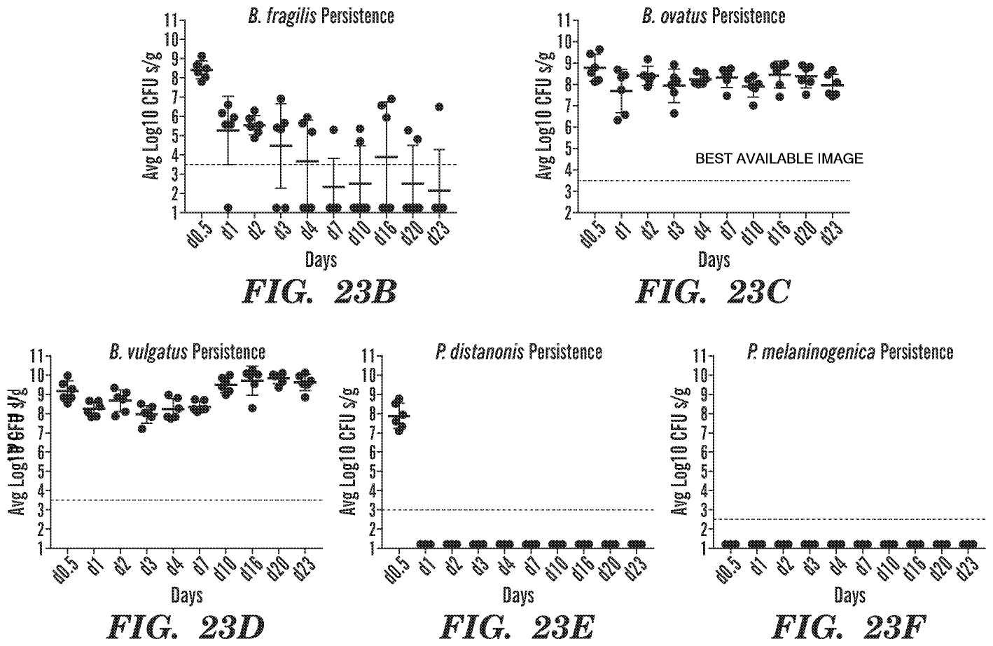

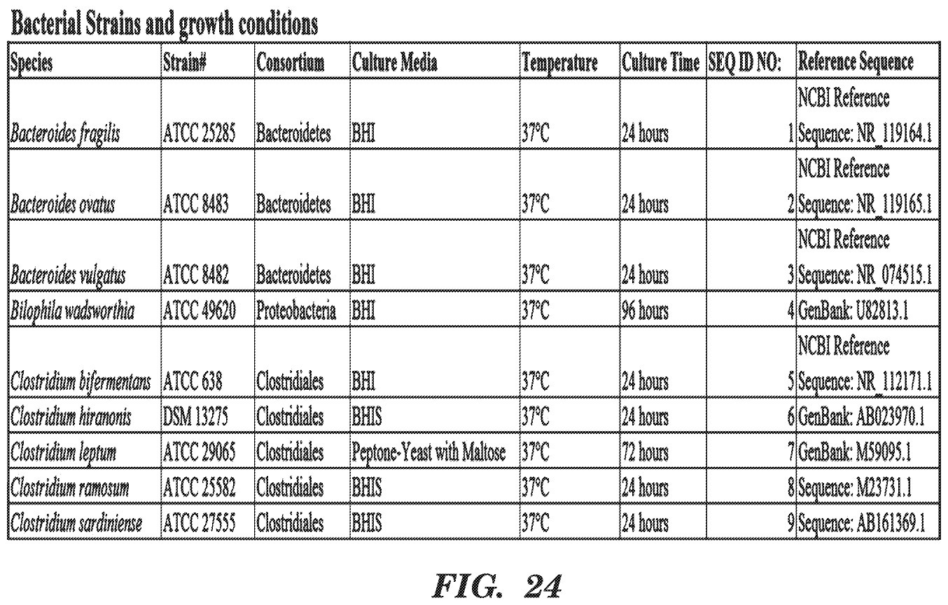

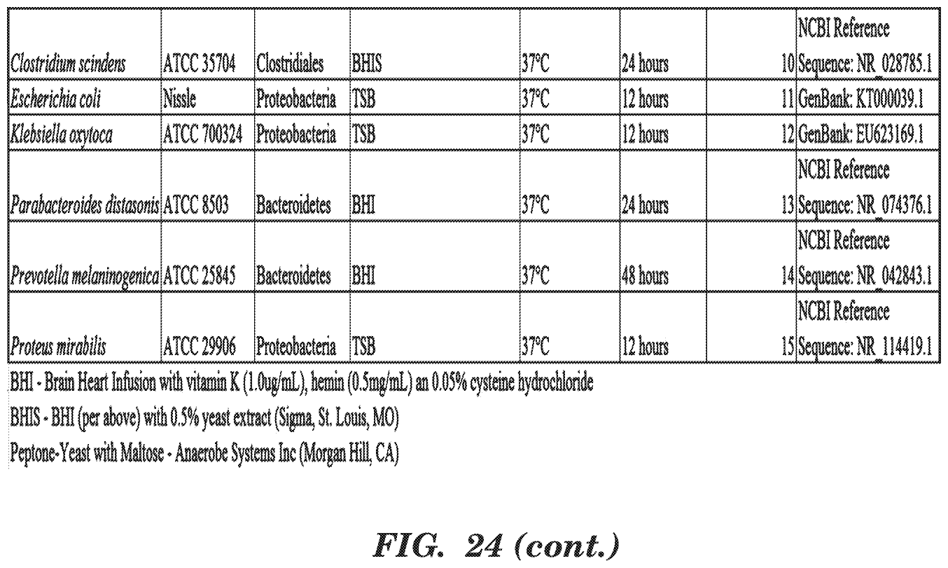

To determine persistence of the consortium members, conventional IL4raF709 mice receiving a single dose of the consortium were followed for 3 weeks with serial collection of fecal samples prior to and after dosing. The dose contained approximately 1×10⁷ CFU/g of each organism. Specific quantitative qPCR probes for each species, and that did not cross-react with conventional microbiota (FIG. 23A), were used to assess molecular biomass of the organisms administered in the consortium dose. As shown in FIG. 23B-23F, B. vulgatus and B. ovatus persisted in all mice during the sampling period. In more than half of mice, B. fragilis was detectable. P. distasonis was detectable at 12 hours but not thereafter. P. melaninogenica was not detectable at 12 hours after administration (gut transit time in adult mice is 6-7 hours).

SEQUENCES

```
SEQ ID NO: 1 (Bacteroides fragilis strain ATCC 25285
16S ribosomal RNA, partial sequence)
NCBI Reference Sequence: NR_119164.1
      1 ATGAACGCTA GCTACAGGCT TAACACATGC AAGTCGAGGG GCATCAGGAA GAAAGCTTGC
    61 TTTCTTTGCT GGCGACCGGC GCACGGGTGA GTAACACGTA TCCAACCTGC CCTTTACTCG
  121 GGGATAGCCT TTCGAAAGAA AGATTAATAC CCGATAGCAT AATGATTCCG CATGGTTTCA
  181 TTATTAAAGG ATTCCGGTAA AGGATGGGGA TGCGTTCCAT TAGGTTGTTG GTGAGGTAAC
  241 GGCTCACCAA GCCTTCGATG GATAGGGGTT CTGAGAGGAA GGTCCCCCAC ATTGGAACTG
  301 AGACACGGTC CAAACTCCTA CGGGAGGCAG CAGTGAGGAA TATTGGTCAA TGGGCGCTAG
  361 CCTGAACCAG CCAAGTAGCG TGAAGGATGA AGGCTCTATG GGTCGTAAAC TTCTTTTATA
  421 TAAGAATAAA GTGCAGTATG TATACTGTTT TGTATGTATT ATATGAATAA GGATCGGCTA
  481 ACTCCGTGCC AGCAGCCGCG GTAATACGGA GGATCCGAGC GTTATCCGGA TTTATTGGGT
  541 TTAAAGGGAG CGTAGGTGGA CTGGTAAGTC AGTTGTGAAA GTTTGCGGCT CAACCGTAAA
  601 ATTGCAGTTG ATACTGTCAG TCTTGAGTAC AGTAGAGGTG GGCGGAATTC GTGGTGTAGC
  661 GGTGAAATGC TTAGATATCA CGAAGAACTC CGATTGCGAA GGCAGCTCAC TGGACTGCAA
  721 CTGACACTGA TGCTCGAAAG TGTGGGTATC AAACAGGATT AGATACCCTG GTAGTCCACA
  781 CAGTAAACGA TGAATACTCG CTGTTTGCGA TATACAGTAA GCGGCCAAGC GAAAGCATTA
  841 AGTATTCCAC CTGGGGAGTA CGCCGGCAAC GGTGAAACTC AAAGGAATTG ACGGGGGCCC
  901 GCACAAGCGG AGGAACATGT GGTTTAATTC GATGATACGC GAGGAACCTT ACCCGGGCTT
  961 AAATTGCAGT GGAATGATGT GGAAACATGT CAGTGAGCAA TCACCGCTGT GAAGGTGCTG
1021 CATGGTTGTC GTCAGCTCGT GCCGTGAGGT GTCGGCTTAA GTGCCATAAC GAGCGCAACC
1081 CTTATCTTTA GTTACTAACA GGTTATGCTG AGGACTCTAG AGAGACTGCC GTCGTAAGAT
1141 GTGAGGAAGG TGGGGATGAC GTCAAATCAG CACGGCCCTT ACGTCCGGGG CTACACACGT
1201 GTTACAATGG GGGGTACAGA AGGCAGCTAG CGGGTGACCG TATGCTAATC CCAAAATCCT
1261 CTCTCAGTTC GGATCGAAGT CTGCAACCCG ACTTCGTGAA GCTGGATTCG CTAGTAATCG
1321 CGCATCAGCC ACGGCGCGGT GAATACGTTC CCGGGCCTTG TACACACCGC CCGTCAAGCC
1381 ATGGGAGCCG GGGGTACCTG AAGTACGTAA CCGCAAGGAT CGTCCTAGGG TAAAAC
SEQ ID NO: 2 (Bacteroides ovatus strain ATCC 8483 16S ribosomal RNA,
partial sequence)
NCBI Reference Sequence: NR_119165.1
      1 ATGAACGCTA GCTACAGGCT TAACACATGC AAGTCGAGGG GCAGCATTTT NGTTTGCTTG
    61 CAAACTGAAG ATGGCGACCG GCGCACGGGT GAGTAACACG TATCCAACCT GCCGATAACT
  121 CCGGNATAGC CTTTCGAAAG AAAGATTAAT ACCNGATAGC ATACGAANAN CGCATGNTAN
  181 TTTTATTAAA GAATTTCGGT TATCGATGGG GATGCGTTCC ATTAGTTTGT TGGCGGGGTA
  241 ACGGCCCACC AAGACTACGA TGGATAGGGG TTCTGAGAGG AAGGTCCCCC ACATTGGAAC
  301 TGAGACACGG TCCAAACTCC TACGGGAGGC AGCAGTGAGG AATATTGGTC AATGGGCGAG
  361 AGCCTGAACC AGCCAAGTAG CGTGAAGGAT GANGGCCCTA TGGGTCGTAA ACTTCTTTTA
  421 TATGGGAATA AAGTNTTCCA CGTGTGGAAT TTTGTATGTA CCATATGAAT AAGGATCGGC
  481 TAACTCCGTG CCAGCAGCCG CGGTAATACG GAGGATCCGA GCGTTATCCG GATTTATTGG
  541 GTTTAAAGGG AGCGTAGGTG GATTGTTAAG TCAGTTGTGA AAGTTTGCGG CTCAACCGTA
  601 AAATTGCAGT TGAAACTGGC AGTCTTGAGT ACAGTAGAGG TGGGCGGAAT TCGTGGTGTA
  661 GCGGTGAAAT GCTTAGATAT CACGAAGAAC TCCGATTGCG AAGGCAGCTC ACTAGACTGN
  721 NACTGACACT GATGCTCGAA AGTGTGGGTA TCAAACAGGA TTAGATACCC TGGTAGTCCA
  781 CACAGTAAAC GATGAATACT CGCTGTTTGC GATATACAGT AAGCGGCCAA GCGAAAGCAT
  841 TAAGTATTCC ACCTGGGGAG TACGCCGGCA ACGGTGAAAC TCAAAGGAAT TGACGGGGGC
  901 CNGCACAAGC GGAGGAACAT GTGGTTTAAT TCGATGATAC GCGAGGAACC TTACCCGGGC
  961 TTAAATTGCA ACNGAATATA TTGGAAACAG TATAGCCGNA AGGCTGTTGT GAAGGTGCTG
1021 CATGGTTGTC GTCAGCTCGT GCCGTGAGGT GTCGGCTTAA GTGCCATAAC GAGCGCAACC
1081 CNTATCTTTA GTTACTAACA GGTTATGCTG AGGACTCTAG AGAGACTGCC GTCGTAAGAT
1141 GTGAGGAAGG TGGGGATGAC GTCAAATCAG CACGGCCCTT ACGTCCGGGG CTACACACGT
1201 GTTACAATGG GGGGTACAGA AGGCAGCTAC CNGGNGACAG GATGCTAATC CCAAAAACCT
1261 CTCTCAGTTC GGATCGAAGT CTGCAACCCG ACTTCGTGAA GCTGGATTCG CTAGTAATCG
1321 CGCATCAGCC ATGGCGCGGT GAATACGTTC CCGGGCCTTG TACACACCGC CCGTCAAGCC
1381 ATGAAAGCCG GGGGT
SEQ ID NO: 3 (Bacteroides vulgatus strain ATCC 8482 16S ribosomal RNA,
partial sequence)
NCBI Reference Sequence: NR_074515.1
      1 TATTACAATG AAGAGTTTGA TCCTGGCTCA GGATGAACGC TAGCTACAGG CTTAACACAT
    61 GCAAGTCGAG GGGCAGCATG GTCTTAGCTT GCTAAGGCCG ATGGCGACCG GCGCACGGGT
  121 GAGTAACACG TATCCAACCT GCCGTCTACT CTTGGACAGC CTTCTGAAAG GAAGATTAAT
  181 ACAAGATGGC ATCATGAGTC CGCATGTTCA CATGATTAAA GGTATTCCGG TAGACGATGG
  241 GGATGCGTTC CATTAGATAG TAGGCGGGGT AACGGCCCAC CTAGTCTTCG ATGGATAGGG
  301 GTTCTGAGAG GAAGGTCCCC CACATTGGAA CTGAGACACG GTCCAAACTC CTACGGGAGG
  361 CAGCAGTGAG GAATATTGGT CAATGGGCGA GAGCCTGAAC CAGCCAAGTA GCGTGAAGGA
  421 TGACTGCCCT ATGGGTTGTA AACTTCTTTT ATAAAGGAAT AAAGTCGGGT ATGGATACCC
  481 GTTTGCATGT ACTTTATGAA TAAGGATCGG CTAACTCCGT GCCAGCAGCC GCGGTAATAC
  541 GGAGGATCCG AGCGTTATCC GGATTTATTG GGTTTAAAGG GAGCGTAGAT GGATGTTTAA
  601 GTCAGTTGTG AAAGTTTGCG GCTCAACCGT AAAATTGCAG TTGATACTGG ATATCTTGAG
  661 TGCAGTTGAG GCAGGCGGAA TTCGTGGTGT AGCGCTGAAA TgCTTAGATA TCACGAAGAA
  721 CTCCGATTGC GAAGGCAGCC TGCTAAGCTG CAACTGACAT TGAGGCTCGA AAGTGTGGGT
  781 ATCAAACAGG ATTAGATACC CTGGTAGTCC ACACGGTAAA CGATGAATAC TCGCTGTTTG
  841 CGATATACTG CAAGCGGCCA AGCGAAAGCG TTAAGTATTC CACCTGGGGA GTACGCCGGC
  901 AACGGTGAAA CTCAAAGGAA TTGACGGGGG CCCGCACAAG CGGAGGAACA TGTGGTTTAA
  961 TTCGATGATA CGCGAGGAAC CTTACCCGGG CTTAAATTGC AGATGAATTA CGGTGAAAGC
1021 CGTAAGCCGC AAGGCATCTG TGAAGGTGCT GCATGGTTGT CGTCAGCTCG TGCCGTGAGG
1081 TGTCGGCTTA AGTGCCATAA CGAGCGCAAC CCTTGTTGTC AGTTACTAAC AGGTTCCGCT
1141 GAGGACTCTG ACAAGACTGC CATCGTAAGA TGTGAGGAAG GTGGGGATGA CGTCAAATCA
1201 GCACGGCCCT TACGTCCGGG GCTACACACG TGTTACAATG GGGGGTACAG AGGGCCGCTA
1261 CCACGCGAGT GGATGCCAAT CCCCAAAACC TCTCTCAGTT CGGACTGGAG TCTGCAACCC
1321 GACTCCACGA AGCTGGATTC GCTAGTAATC GCGCATCAGC CACGGCGCGG TGAATACGTT
1381 CCCGGGCCTT GTACACACCG CCCGTCAAGC CATGGGAGCC GGGGGTACCT GAAGTGCGTA
1441 ACCGCGAGGA GCGCCCTAGG GTAAAACTGG TGACTGGGGC TAAGTCGTAA CAAGGTAGCC
1501 GTACCGGAAG
SEQ ID NO: 4 (Bilophila wadsworthia 16S ribosomal RNA gene, partial
sequence)
GenBank: U82813.1
      1 CTTAACACAT GCAAGTCGAA CGTGAAAGTC CTTCGGGATG AGTAAAAGTG GCGCACGGGT
    61 GAGTAACGCG TGGATAATCT ACCCTTAAGA TGGGGATAAC GGCTGGAAAC GGTCGCTAAT
  121 ACCGAATACG CTCCCGATTT TATCATTGGG GGGAAAGATG GCCTCTGCTT GCAAGCTATC
  181 GCTTAAGGAT GAGTCCGCGT CCCATTAGCT AGTTGGCGGG GTAACGGCCC ACCAAGGCAA
  241 CGATGGGTAG CCGGTCTGAG AGGATGACCG GCCACACTGG AACTGGAACA CGGTCCAGAC
  301 TCCTACGGGA GGCAGGAGTG GGGAATATTG CGCAATGGGC GAAAGCCTGA CGCAGCGACG
  361 CCGCGTGAGG GATGAAGGTT CTCGGATCGT AAACCTCTOT CAGGGSGGAA GAAACCCCCT
  421 CGTGTGAATA ATGCGAGGGC TTGACGGTAC CCCCAAAGGA AGCACCGGCT AACTCCGTGC
  481 CAGCAGCCGC GGTAATACGG AGGGTGCAAG CGTTAATCGG AATCACTGGG CGTAAAGCGC
  541 ACGTACGCGG CTTGGTAAGT CAGGGGTGAA ATCCCACAGC CCAACTGTGG AACTGCCTTT
  601 GATACTGCCA CGCTTGAGTA CCGGAGAGGG TGGCGGAATT CCAGGTGTAG GAGTGAAATC
  661 CGTAGATATC TGGAGGAACA CCGGTGGCGA AGGCGGCCAC CTGGACGGTA ACTGACGCTG
  721 AGGTGCGAAA GCGTGGGTAG CAAACAGGAT TAGATACCCT GGTAGTCCAC GCTGTAAACG
  781 ATGGGTGCNG GGTGCTGGGA TGTATGTCTC GGTGCCGTAG CTAACGCGAT AAGCACCCCG
  841 CCTGGGGAGT ACGGTCGCAA GGCTGAAACT CAAAGAAATT GACGGGGGCC CGCACAAGCG
  901 GTGGAGTATG TGGTTTAATT CGATGCAACG CGAAGAACCT TACCCAGGCT TGACATCTAG
  961 GGAACCCTTC GGAAATGAAG GGGTGCCCTT CGGGGAGCCC TAAGACAGGT GCTGCATGGC
1021 TGTCGTCAGC TCGTGCCGTG AGGTGTTGGG TTAAGTCCCG CAACGAGCGC AACCCCTATC
1081 TTCAGTTGCC AGCAGGTAAG GCTGGGCACT CTGGAGAGAC CGCCCCGGTC AACGGGGAGG
1141 AAGGTGGGGA CGACGTCAAG TCATCATGGC CCTTACGCCT GGGGCTACAC ACGTACTACA
1201 ATGGCGCGCA CAAAGGGTAG CGAGACCGCG AGGTGGAGCC AATCCCAAAA AACGCGTCCC
1261 AGTCCGGATT GGAGTCTGCA ACTCGACTCC ATGAAGTCGG AATCGCTAGT AATTCGAGAT
1321 CAGCATGCTC GGGTGAATGC GTTCCCGGGC CTTGTACACA CCGCCCGTCA CACCACGAAA
1381 GTCGGTTTTA CCCGAAGCCG GTGAGCTAAC TCGCAAGAGG AGCAGCCGTC TACGGTAGGG
1441 CCGATGATTG GGGTGAAGTC GTAACAA
SEQ ID NO: 5 ([Clostridium] bifermentans strain ATCC 638 16S ribosomal
RNA gene, partial sequence)
NCBI Reference Sequence: NR_112171.1
      1 CATRGCTCAG GATGAACGCT GGCGGCGTGC CTAACACATG CAAGTCGAGC GATCTCTTCG
    61 GAGAGAGCGG CGGACGGGTG AGTAACGCGT GGGTAACCTG CCCTGTACAC ACGGATAACA
  121 TACCGAAAGG TATACTAATA CGGGATAACA TATGAAAGTC GCATGGCTTT TGTATCAAAG
  181 CTCCGGCGGT ACAGGATGGA CCCGCGTCTG ATTAGCTAGT TGGTAAGGTA ATGGCTTACC
  241 AAGGCAACGA TCAGTAGCCG ACCTGAGAGG GTGATCGGCC ACACTGGAAC TGAGACACGG
  301 TCCAGACTCC TACGGGAGGC AGCAGTGGGG AATATTGCAC AATGGGCGAA AGCCTGATGC
  361 AGCAACGCCG CGTGAGCGAT GAAGGCCTTC GGGTCGTAAA GCTCTGTCCT CAAGGAAGAT
  421 AATGACGGTA CTTGAGGAGG AAGCCCCGGC TAACTACGTG CCAGCAGCCG CGGTAATATG
  481 TAGGGGGCTA GCGTTATCCG GAATTACTGG GCGTAAAGGG TGCGTAGGTG GTTTTTTAAG
  541 TCAGAAGTGA AAGGCTACGG CTCAACCGTA GTAAGCTTTT GAAACTAGAG AACTTGAGTG
  601 CAGGAGAGGA GAGTAGAATT CCTAGTGTAG CGGTGAAATG CGTAGATATT AGGAGGAATA
  661 CCAGTAGCGA AGGCGGCTCT CTGGACTGTA ACTGACACTG AGGCACGAAA GCGTGGGGAG
  721 CAAACAGGAT TAGATACCCT GGTAGTCCAC GCCGTAAACG ATGAGTACTA GGTGTCGGGG
  781 GTTACCCCCC TCGGTGCCGC ACTAACGCAT TAAGTACTCC GCCTGGGAAG TACGCTCGCA
  841 AGAGTGAAAC TCAAAGGAAT TTDCGGGGAC CCGCACAAGT AGCGGAGCAT GTGGTTTAAT
  901 TCGAAGCAAC GCGAAGAACC TTACCTAAGC TTGACATCCC AGTGACCTCT CCCTAATCGG
  961 AGATTTCCCT TCGGGGACAG TGGTGACAGG TGGTGCATGG TTGTCGTCAG CTCGTGTCGT
1021 GAGATGTTGG GTTAAGTCCC GCAACGAGCG CAACCCTTGC CTTTAGTTGC CAGCATTAAG
1081 TTGGGCACTC TAGAGGGACT GCCGAGGATA ACTCGGAGGA AGGTGGGGAT GACGTCAAAT
1141 CATCATGCCC CTTATGCTTA GGGCTACACA CGTGCTACAA TGGGTGGTAC AGAGGGTTGC
1201 CAAGCCGCGA GGTGGAGCTA ATCCCTTAAA GCCATTCTCA GTTCGGATTG TAGGCTGAAA
1261 CTCGCCTACA TGAAGCTGGA GTTACTAGTA ATCGCAGATC AGAATGCTGC GGTGAATGCG
1321 TTCCCGGGTC TTGTACACAC CGCCCGTCAC ACCATGGAAG TTGGGGGCGC CCGAAGCCGG
1381 TTAGCTAACC TTTTAGGAAG CGGCCGTCGA AGGTGAACAA ATGACTGGGG TGAAGTCGTA
1441 ACAAGGTANC CGTATCGGAA GGTGCGGCBG GATCAA
SEQ ID NO: 6 (Clostridium hiranonis gene for 16S rRNA, partial
sequence, strain: TO-931)
GenBank: AB023970.1
      1 ACATGCAAGT CGAGCGATTC TCTTCGGAGA AGAGCGGCGG ACGGGTGAGT AACGCGTGGG
    61 TAACCTGCCC TGTACACACG GATAACATAC CGAAAGGTAT GCTAATACGG GATAATATAT
  121 AAGAGTCGCA TGACTTTTAT ATCAAAGATT TTTCGGTACA GGATGGACCC GCGTCTGATT
  181 AGCTTGTTGG CGGGGTAACG GCCCACCAAG GCGACGATCA GTAGCCGACC TGAGAGGGTG
  241 ATCGGCCACA TTGGAACTGA GACACGGTCC AAACTCCTAC GGGAGGCAGC AGTGGGGAAT
  301 ATTGCACAAT GGGCGCAAGC CTGATGCAGC AACGCCGCGT GAGCGATGAA GGCCTTCGGG
  361 TCGTAAAGCT CTGTCCTCAA GGAAGATAAT GACGGTACTT GAGGAGGAAG CCCCGGCTAA
  421 CTACGTGCCA GCAGCCGCGG TAATACGTAG GGGGCTAGCG TTATCCGGAT TTACTGGGCG
  481 TAAAGGGTGC GTAGGCGGTC TTTCAAGTCA GGAGTTAAAG GCTACGGCTC AACCGTAGTA
  541 AGCTCCTGAT ACTGTCTGAC TTGAGTGCAG GAGAGGAAAG CGGAATTCCC AGTGTAGCGG
  601 TGAAATGCGT AGATATTGGG AGGAACACCA GTAGCGAAGG CGGCTTTCTG GACTGTAACT
  661 GACGCTGAGG CACGAAAGCG TGGGGAGCAA ACAGGATTAG ATACCCTGGT AGTCCACGCT
  721 GTAAACGATG AGTACTAGTT GTCGGAGGTT ACCCCTTCGG TGCCGCAGCT AACGCATTAA
  781 GTACTCCGCC TGGGGAGTAC GCACGCAAGT GTGAAACTCA AAGGAATTGA CGGGGACCCG
  841 CACAAGTAGC GGAGCATGTG GTTTAATTCG AAGCAACGCG AAGAACCTTA CCTAGGCTTG
  901 ACATCCTTCT GACCGAGGAC TAATCTCCTC TTTCCCTCCG GGGACAGAAG TGACAGGTGG
  961 TGCATGGTTG TCGTCAGCTC GTGTCGTGAG ATGTTGGGTT AAGTCCCGCA ACGAGCGCAA
1021 CCCTTGTCTT TAGTTGCCAT CATTAAGTTG GGCACTCTAG AGAGACTGCC AGGGATAACC
1081 TGGAGGAAGG TGGGGATGAC GTCAAATCAT CATGCCCCTT ATGCCTAGGG CTACACACGT
1141 GCTACAATGG GTGGTACAGA GGGCAGCCAA GCCGTGAGGT GGAGCAAATC CCTTAAAGCC
1201 ATTCTCAGTT CGGATTGTAG GCTGAAACTC GCCTACATGA AGCTGGAGTT ACTAGTAATC
1261 GCAGATCAGA ATGCTGCGGT GAATGCGTTC CCGGGTCTTG TACACACCGC CCGTCACACC
1321 ATGGGAGTTG GAGACACCCG AAGCCGACTA TCTAACCTTT TGGGAGAAGT CGTCCCCCTC
1381 GAATCAATAC CCC
SEQ ID NO: 7 (Clostridium leptum 16S ribosomal RNA)
GenBank: M59095.1
      1 NNNNNNNNNN NNNNNNNNNN NNNNNNNNNN NNNNNNNNNN NNNNNNNNNN NNNNNNNNNN
    61 NNNNNNNNNN NNNNNNNNNN NNNNTTGGAT TTAACTTAGT GGCGGACGGG TGAGTAACGC
  121 GTGAGTAACC TGCCTTTCAG AGGGGGATAA CGTTCTGAAA AGAACGCTAA TACCGCATAA
  181 CATCAATTTA TCGCATGATA GGTTGATCAA AGGAGCAATC CGCTGGAAGA TGNACTCGCG
  241 TCCGATTAGC CAGTTGGCGG GGTAACGGCC NACCAAAGCG ACGATCGGTA GCCGGACTGA
  301 GAGGTTGAAC GGCCACATTG GGACTGAGAC ACGGCCNNGA CTCCTACGGG AGGCAGCAGT
  361 GGGGGATATT GCACAATGGG GGAAACCCNG ATGCAGCAAC GCCGCGTGAG GGAAGAAGGT
  421 TTTCGGATTG TAAACCTCTG TTCTTAGTGA CGATAATGAC GGTAGCTAAG GAGAAAGCTC
  481 CNNNNAACTA CGTGCCAGCA GCCGCGGTAA TACGTAGGGA GCNAGCGTTG TCCGGATTTA
  541 CTGGGTGTAA AGGGTGCGTA GGCGGCGAGG CAAGTCAGGC GTGAAATCTA TGCGCTTAAC
  601 CCATAAACTG CGCTTGAAAC TGTCTTGCTT GAGTGAAGTA GAGGTAGGCG GAATTCCCNG
  661 TGTAGCGGTN AAATGCGTAG AGATCGGGAG GAACACCAGT GGCGAAGGCG GCCTACTGGG
  721 CTTTAACTGA CGCTGAAGCA CGAAAGCATG GGTAGCAAAC AGGATTAGAT ACCCTGGTAG
  781 TCCATGCCGT AAACGATGAT TACTAGGTGT GNNGGGGGTC TNACCCNNTC CGTGCCGCAG
  841 TTAACACAAT AAGTAATCCA CCTCGGGAGT ACGGCCGCAA GGTTGAAACT CAAAGGAATT
  901 GACGGNNNCC CGCACAAGCA GTGGAGTATG TNGTTTAATT CGAANNAACG CGAAGAACCT
  961 TACCAGGNCT TGACATCCGT CTAACGAAGC AGAGATGCAT TAGGTGCCCT TCGGGGNAAG
1021 GCGAGACAGG TGGTGCATGG TTGTCGTCAG CTCGTGTCGT GAGATGTTGG GTTAAGTCNN
1081 GCAACGAGCG CAACNCTTGT TTCTAGTTGC TACGCAAGAG CACTCTAGAG AGACTGCCGT
1141 TGACAAAACG GAGGAAGGTG GGGACGACGT CAAATCATCA TGCCCNNTAT GACCTGGGCC
1201 ACACACGTAC TACAATGGCT GTANACAGAG GGAAGCAAAG CCGCGAGGTG GAGCAAAACC
1261 CTAAAAGCAG TCCCAGTTCG GATCGCAGGC TGCAACCCGC CTGCGTGAAG TCGGAATTGC
1321 TAGTAATCGC GGATCAGCAT GCCGCGGTGA ATACGTTCCC GGGCNNTGTA CACACCGCCC
1381 GTCACACCAT GGGAGCCGGT AATACCCGAA GCCAGTAGTT CAACCGCAAG GAGAGCGCTG
1441 TCGAAGGTAG GATTGGCGAC NNGGG
SEQ ID NO: 8 (C. ramosum 16S ribosomal RNA small subunit.)
Accesion: M23731.1
      1 ACAATGGAGA GTTTGATCCT GGCTCAGGAT GAACGCTGGC GGCGTGCCTA ATACATGCAA
    60 GTCGAACGCN AGCACTTGTG CTTCGAGTGG CGAACGGGTG AGTAATACAT AAGTAACCTG
  120 CCCTAGACAG GGGGATAACT ATTGGAAACG ATAGCTAAGA CCGCATAGGT ACGGACACTG
  180 CATGGTGACC GTATTAAAAG TGCCTCAAAG CACTGGTAGA GGATGGACTT ATGGCGCATT
  240 AGCTAGTTGG CGGGGTAACG GCCCACCAAG GCGACGATGC GTAGCCGACC TGAGAGGGTG
  300 ACCGGCCACA CTGGGACTGA GACACGGCCC AGACTCCTAC GGGAGGCAGC AGTAGGGAAT
  360 TTTCGGCAAT GGGGGAAACC CTGACCGAGC AACGCCGCGT GAAGGAAGAA GGTTTTCGGA
  420 TTGTAAACTT CTGTTATAAA GGAAGAACGG CGGCTACAGG AAATGGTAGC CGAGTGACGG
  480 TACTTTATTA GAAAGCCACG GCTAACTACG TGCCAGCAGC CGCGGTAATA CGTAGGTGGC
  540 NAGCGTTATC CGGAATTATT GGGCGTAAAG AGGGAGCAGG CGCCAGCAAG GGTCTGTGGT
  600 GAAAGCCTGA AGCTTAACTT CAGTAAGCCA TAGAAACCAG GCAGCTAGAG TGCAGGAGAG
  660 GATCGTGGAA TTCCATGTGT AGCGGTGAAA TGCGTAGATA TATGGAGGAA CACCAGTGGC
  720 GAAGGCGACG ATCTGGCCTG CAACTGACGC TCAGTCCCGA AAGCGTGGGG AGCAAATAGG
  780 ATTAGATACC CTAGTAGTCC ACGCCGTAAA CGATGAGTAC TAAGTGTTGG ATGTCAAAGT
  840 TCAGTGCTGC AGTTAACGCA ATAAGTACTC CGCCTGAGTA GTACGTTCGC AAGAATGAAA
  900 CTCAAAGGAA TTGACGGGGG CCCGCACAAG CGGTGGAGCA TGTGGTTTAA TTCGAAGCAA
  960 CGCGAAGAAC CTTACCAGGT CTTGACATAC TCATAAAGGC TCCAGAGATG GAGAGATAGC
1020 TATATGAGAT ACAGGTGGTG CATGGTTGTC GTCAGCTCGT GTCGTGAGAT GTTGGGTTAA
1080 GTCCCGCAAC GAGCGCAACC CTTATCGTTA GTTACCATCA TTAAGTTGGG GACTCTAGCG
1140 AGACTGCCAG TGACAAGCTG GAGGAAGGCG GGGATGACGT CAAATCATCA TGCCCCTTAT
1200 GACCTGGGCT ACACACGTGC TACAATGGAT GGTGCAGAGG GAAGCGAAGC CGCGAGGTGA
1260 AGCAAAACCC ATAAAACCAT TCTCAGTTCG GATTGTAGTC TGCAACTCGA CTACATGAAG
1320 TTGGAATCGC TAGTAATCGC GAATCAGCAT GTCGCGGTGA ATACGTTCTC GNGCCTTGTA
1380 CACACCGCCC GTCACACCAC GAGAGTTGAT AACACCCGAA GCNGGTGGCC TAACCGCAAG
1440 GAAGGAGCTG TCTAAGGTGG GATTGATGAT NGGGGNNNNN NNGTAACAAG GTATCCCTAC
1500 GNGAACGNNN NNNNNGATCA CCTCCTTTCN
SEQ ID NO: 9 (Clostridium sardiniense gene for 16S rRNA, strain:
DSM 599, sub_clone:c1.)
Sequence: AB161369.1
TTTAAATTGAGAGTTTGATCCTGGCTCAGGACGAACGCTGGCGGCGTGCCTAACACATGCAAGTCGAGCG
ATGAAGTTTCCTTCGGGAAACGGATTAGCGGCGGACGGGTGAGTAACACGTGGGTAACCTGCCTCATAGA
GGGGAATAGCCTTCCGAAAGGAAGATTAATACCGCATAACATTGCAGTTTCGCATGAAACAGCAATTAAA
GGAGCAATCCGCTATGAGATGGACCCGCGGCGCATTAGCTAGTTGGTAAGGTAATGGCTTACCAAGGCGA
CGATGCGTAGCCGACCTGAGAGGGTGATCGGCCACATTGGGACTGAGACACGGCCCAGACTCCTACGGGA
GGCAGCAGTGGGGAATATTGCACAATGGGGGAAACCCTGATGCAGCAACGCCGCGTGAGTGATGACGGTC
TTCGGATTGTAAAGCTCTGTCTTTGGGGACGATAATGACGGTACCCAAGGAGGAAGCCACGGCTAACTAC
GTGCCAGCAGCCGCGGTAATACGTAGGTGGCAAGCGTTGTCCGGATTTACTGGGCGTAAAGGGAGCGTAG
GCGGATTTTTAAGTGGGATGTGAAATACCCGGGCTCAACCTGGGTGCTGCATTCCAAACTGGGAATCTAG
AGTGCAGGAGGGGAGAGTGGAATTCCTAGTGTAGCGGTGAAATGCGTAGAGATTAGGAAGAACACCAGTG
GCGAAGGCGACTCTCTGGACTGTAACTGACGCTGAGGCTCGAAAGCGTGGGGAGCAAACAGGATTAGATA
CCCTGGTAGTCCACGCCGTAAACGATGAATACTAGGTGTAGGGGTTTCGATACCTCTGTGCCGCCGCTAA
CGCATTAAGTATTCCGCCTGGGGAGTACGGTCGCAAGATTAAAACTCAAAGGAATTGACGGGGGCCCGCA
CAAGTAGCGGAGCATGTGGTTTAATTCGAAGCAACGCGAAGAACCTTACCTAGACTTGACATCTTCTGCA
TTACCCTTAATCGGGGAAGTCCTTTCGGGGACAGAATGACAGGTGGTGCATGGTTGTCGTCAGCTCGTGT
CGTGAGATGTTGGGTTAAGTCCCGCAACGAGCGCAACCCCTATTGTTAGTTGCTACCATTAAGTTGAGCA
CTCTAGCGAGACTGCCCGGGTTAACCGGGAGGAAGGTGGGGATGACGTCAAATCATCATGCCCCTTATGT
CTAGGGCTACACACGTGCTACAATGGCAAGTACAGAGAGATGCAATACCGTGAGGTGGAGCTAAACTTCA
AAACTTGTCTCAGTTCGGATTGTAGGCTGAAACTCCCCTACATGAAGCTGGAGTTACTAGTAATCGCGAA
TCAGCATGTCGCGGTGAATACGTTCCCGGGCCTTGTACACACCGCCCGTCACACCATGAGAGTTGGCAAT
ACCCAAAGTTCGTGAGCTAACGCGTAAGCGAGGCAGCGACCTAAGGTAGGGTCAGCGATTGGGGTGAAGT
CGTAACAAGGTAGCCGTAGGAGAACCTGCGGCTGGATCACCTCCTTTCT
SEQ ID NO: 10 ([Clostridium] scindens strain ATCC 35704 16S ribosomal
RNA, partial sequence
NCBI Reference Sequence: NR_028785.1
      1 GAGAGTTTGA TCCTGGCTCA GGATGAACGC TGGCGGCGTG CCTAACACAT GCAAGTCGAA
    61 CGAAGCGCCT GGCCCCGACT TCTTCGGAAC GAGGAGCCTT GCGACTGAGT GGCGGACGGG
  121 TGAGTAACGC GTGGGCAACC TGCCTTGCAC TGGGGGATAA CAGCCAGAAA TGGCTGCTAA
  181 TACCGCATAA GACCGAAGCG CCGCATGGCG CGGCGGCCAA AGCCCCGGCG GTGCAAGATG
  241 GGCCCGCGTC TGATTAGGTA GTTGGCGGGG TAACGGCCCA CCAAGCCGAC GATCAGTAGC
  301 CGACCTGAGA GGGTGACCGG CCACATTGGG ACTGAGACAC GGCCCAGACT CCTACGGGAG
  361 GCAGCAGTGC GGAATATTGC ACAATGGGGG AAACCCTGAT GCAGCGACGC CGCGTGAAGG
  421 ATGAAGTATT TCGGTATGTA AACTTCTATC AGCAGGGAAG AAGATGACGG TACCTGACTA
  481 AGAAGCCCCG GCTAACTACG TGCCAGCAGC CGCGGTAATA CGTAGGGGGC AAGCGTTATC
  541 CGGATTTACT GGGTGTAAAG GGAGCGTAGA CGGCGATGCA AGCCAGATGT GAAAGCCCGG
  601 GGCTCAACCC CGGGACTGCA TTTGGAACTG CGTGGCTGGA GTGTCGGAGA GGCAGGCGGA
  661 ATTCCTAGTG TAGCGGTGAA ATGCGTAGAT ATTAGGAGGA ACACCAGTGG CGAAGGCGGC
  721 CTGCTGGACG ATGACTGACG TTGAGGCTCG AAAGCGTGGG GAGCAAACAG GATTAGATAC
  781 CCTGGTAGTC CACGCCGTAA ACGATGACTA CTAGGTGTCG GGTGGCAAGG CCATTCGGTG
  841 CCGCAGCAAA CGCAATAAGT AGTCCACCTG GGGAGTACGT TCGCAAGAAT GAAACTCAAA
  901 GGAATTGACG GGGACCCGCA CAAGCGGTGG AGCATGTGGT TTAATTCGAA GCAACGCGAA
  961 GAACCTTACC TGATCTTGAC ATCCCGATGC CAAAGCGCGT AACGCGCTCT TTCTTCGGAA
1021 CATCGGTGAC AGGTGGTGCA TGGTTGTCGT CAGCTCGTGT CGTGAGATGT TGGGTTAAGT
1081 CCCGCAACGA GCGCAACCCC TATCTTCAGT AGCCAGCATT TTGGATGGGC ACTCTGGAGA
1141 GACTGCCAGC GAGAACCTGG AGGAAGGTGG GGATGACGTC AAATCATCAT GCCCCTTATG
1201 ACCAGGGCTA CACACGTGCT ACAATGGCGT AAACAAAGGG AGGCGAACCC GCGAGGGTGG
1261 GCAAATCCCA AAAATAACGT CTCAGTTCGG ATTGTAGTCT GCAACTCGAC TACATGAAGT
1321 TGGAATCGCT AGTAATCGCG AATCAGAATG TCGCGGTGAA TACGTTCCCG GGTCTTGTAC
1381 ACACCGCCCG TCACACCATG GGAGTCAGTA ACGCCCGAAG CCGGTGACCC AACCCGTAAG
1441 GGAGGGAGCC GTCGAAGGTG GGACCGATAA CTGGGGTGAA GTCGTAACAA GGTAGCCGTA
1501 TCGGAAGGTG CGGCTGGATC ACCTCCTTC
SEQ ID NO: 11 (Escherichia coli Nissle 1917 strain URCS8 16S ribosomal
RNA gene, partial sequence)
GenBank: KT000039.1
    1 GCTTGCTCCA CCGGAAAAAG AAGAGTGGCG AACGGGTGAG TAACACGTGG GTAACCTGCC
  61 CATCAGAAGC GGATAACACT TGGAAACAGG TGCTAATACC GTATAACAAT CGAAACCOCA
121 TGGTTTTGAT TTGAAAGGCG CTTTCGGGTG TCGCTGATGG ATGGACCCGC GGTGCATTAG
181 CTAGTTGGTG AGGTAACGGC TCACCAAGGC CACGATGCAT AGCCGACCTG AGAGGGTGAT
241 CGGCCACATT GGGACTGAGA CACGGCCCAA ACTCCTACGG GAGGCAGCAG TAGGGAATCT
301 TCGGCAATGG ACGAAAGTCT GACCGAGCAA CGCCGCGTGA GTGAAGAAGG TTTTCGGATC
361 GTAAAACTCT GTTGTTAGAG AAGAACAAGG ATGAGAGTAA CTGTTCATCC CTTGACGGTA
421 TCTAACCAGA AAGCCACGGC TAACTACGTG CCAGCAGCCG CGGTAATACG TAGGTGGCAA
481 GCGTTGTCCG GATTTATTGG GCGTAAAGCG AGCGCAGGCG GTTTCTTAAG TCTGATGTGA
541 AAGCCCCCGG CTCAACCGGG GAGGGTCATT GGAAACTGGG AGACTTGAGT GCAGAAGAGG
601 ACAGTGGAAT TCCATGTGTA GCGCTGAAAT CCGTAGATAT ATGGAGGAAC ACCAGTGCCG
661 AAGGCGGCTC TCTGGTCTGT AACTGACGCT GAGGCTCGAA AGCGTGGGGA GCAAACAGGA
721 TTAGATACCC TGGTAGTCCA CGCCGTAAAC GATGAGTGCT AAGTGTTGGA GGGTTTCCGC
781 CCTTCAGTGC TGCAGCTAAC GCATTAAGCA CTCCGCCTGG GGAGTACGAC CGCAAGGTTG
841 AAAC
SEQ ID NO: 12 (Klebsiella oxytoca culture-collection ATCC: 700324
clone d08 16S-23S ribosomal RNA intergenic spacer, partial sequence;
and tRNA-Ile and tRNA-Ala genes, complete sequence)
GenBank: EU623169.1
    1 CCTGAAAGAA CCTGCCTTTG TAGTGCTCAC ACAGATTGTC TGATGAAAAA TAAGCAGTAA
  61 GAAAATCTCT GCAGGCTTGT AGCTCAGGTG GTTAGAGCGC ACCCCTGATA AGGGTGAGGT
121 CGGTGGTTCA AGTCCACTCA GGCCTACCAA ATTTCTGCTG ATGCTGCGTT GCGGCGACAC
181 TCACATACTT TAAGTATGTT TCGTGTCACC ACGCCTTGCC TCAACAGAAA TTAAGGTTGA
241 TGAGATTTTA ACTACGATGG GGCTATAGCT CAGCTGGGAG AGCGCCTGCT TTGCACGCAG
301 GAGGTCTGCC GTTCGATCCC GCATAGCTCC ACCATCATTA CTGCCAAAAA CAAGAAAACT
361 TCAGAGTGTA CCTGAAAAGG TTCACTGCGA AGTTTTGCTC TTTAAAAATC TGGATCAAGC
421 TGAAAATTGA AACGACACAC AGCTAATGTG TGTTCGAGTC TCTCAAATTT TCGCGACACG
481 ATGATGTTTC ACGAAACATC TTCGGGTTGT GA
SEQ ID NO: 13 (Parabacteroides distasonis strain ATCC 8503 16S
ribosomal RNA, partial sequence)
NCBI Reference Sequence: NR_074376.1
      1 CAATTTAAAC AACGAAGAGT TTGATCCTGG CTCACGATCA ACGCTAGCGA CACGCTTAAC
    61 ACATGCAAGT CGAGGGGCAG CGGGGTGTAG CAATACACCG CCGGCGACCG GCGCACGGGT
  121 GAGTAACGCG TATGCAACTT GCCTATCAGA GGGGGATAAC CCGGCGAAAG TCGGACTAAT
  181 ACCGCATGAA GCAGGGATCC CGCATGGGAA TATTTGCTAA AGATTCATCG CTGATAGATA
  241 GGCATGCGTT CCATTAGGCA GTTGGCGGGG TAACGGCCCA CCAAACCGAC GATGGATAGG
  301 GGTTCTGAGA GGAAGGTCCC CCACATTGGT ACTGAGACAC GGACCAAACT CCTACGGGAG
  361 GCAGCAGTGA GGAATATTGG TCAATGGCCG AGAGGCTGAA CCAGCCAAGT CGCGTGAGGG
  421 ATGAAGGTTC TATGGATCGT AAACCTCTTT TRTAAGGGAA TARAGTGCGG GACGTGTCCC
  481 GTTTTGTATG TACCTTATGA ATAAGGATCG GCTAACTCCG TGCCAGCAGC CGCGGTAATA
  541 CGGAGGATCC GAGCGTTATC CGGATTTATT GGGTTTAAAG GGTGCGTAGG CGGCCTTTTA
  601 AGTCAGCGGT GAAAGTCTGT GGCTCAACCA TAGAATTGCC GTTGAAACTG GGGGGCTTGA
  661 GTATGTTTGA GGCAGGCGGA ATGCGTGGTG TAGCGGTGAA ATGCATAGAT ATCACGCAGA
  721 ACCCCGATTG CGAAGGCAGC CTGCCAAGCC ATTACTGACG CTGATGCACG AAAGCGTGGG
  781 GATCAAACAG GATTAGATAC CCTGGTAGTC CACGCAGTAA ACGATGATCA CTAGCTGTTT
  841 GCGATACACT GTAAGCGGCA CAGCGAAAGC GTTAAGTGAT CCACCTGGGG AGTACGCCGG
  901 CAACGGTGAA ACTCAAAGGA ATTGACGGGG GCCCGCACAA GCGGAGGAAC ATGTGGTTTA
  961 ATTCGATGAT ACGCGAGGAA CCTTACCCGG GTTTGAACGC ATTCGGACCG AGGTGGAAAC
1021 ACCTTTTCTA GCAATAGCCG TTTGCGAGGT GCTGCATGGT TGTCGTCAGC TCGTGCCGTG
1081 AGGTGTCGGC TTRAGTGCCA TAACGAGCGC ARCCCTTGCC ACTAGTTACT AACAGGTTAG
1141 GCTGAGGACT CTGGTGGGAC TGCCAGCGTA AGCTGCGAGG AAGGCGGGGA TGACGTCAAA
1201 TCAGCACGGC CCTTACATCC GGGGCGACAC ACGTGTTACA ATGGCGTGGA CAAAGGGAGG
1261 CCACCTGGCG ACAGGGAGCG AATCCCCAAA CCACGTCTCA GTTCGGATCG GAGTCTGCAA
1321 CCCGACTCCG TGAAGCTGGA TTCGCTAGTA ATCGCGCATC AGCCATGGCG CGGTGAATAC
1381 GTTCCCGGGC CTTGTACACA CCGCCCGTCA AGCCATGGGR GCCGGGGGTA CCTGAAGTCC
1441 GTAACCGAAA GGATCGGCCT AGGGTAAAAC TGGTGACTGG GGCTAAGTCG TAACAAG
SEQ ID NO: 14 (Prevotella melaninogenica strain ATCC 25845 16S
ribosomal RNA gene, partial sequence)
NCBI Reference Sequence: NR_042843.1
      1 GATGAACGCT AGCTAGAGGC TTAACACATG CAAGTNGAGG GGAAACGGCA TTGAGTGCTT
    61 GCACTCTTTG GACGTCGACC GGCGCACGGG TGAGTAACGC GTATCCAACC TTCCCATTAC
  121 TGTGGGATAA CCTGCCGAAA GGCAGACTAA TACCGCATAG TCTTCGATGA CGGCATCAGA
  181 TTTGAAGTAA AGATTTATCG GTAATGGATG GGGATGCGTC TGATTAGCTT GTTGGCGGGG
  241 TAACGGCCCA CCAAGGCAAC GATCAGTAGG GGTTCTGAGA GGAAGGTCCC CCACATTGGA
  301 ACTGAGACAC GGTCCAAACT CCTACGGGAG GCAGCAGTGA GGAATATTGG TCAATGGACG
  361 GAAGTCTGAA CCAGCCAAGT AGCGTGCAGG ATGACGGCCC TATGGGTTGT AAACTGCTTT
  421 TGTATGGGGA TAAAGTTAGG GACGTGTCCC TATTTGCAGG TACCATACGA ATAAGGACCG
  481 GCTAATTCCG TGCCAGCAGC CGCGGTAATA CGGAAGGTCC AGGCGTTATC CGGATTTATT
  541 GGGTTTAAAG GGAGCGTAGG CTGGAGATTA AGTGTGTTGT GAAATGTAGA CGCTCAACGT
  601 CTGAATTGCA GCGCATACTG GTTTCCTTGA GTACGCACAA CGTTGGCGGA ATTCGTCGTG
  661 TAGCGGTGAA ATGCTTAGAT ATGACGAAGA ACTCCGATTG CGAAGGCAGC TGACGGGAGC
  721 GCAACTGACG CTTAAGCTCG AAGGTGCGGG TATCAAACAG GATTAGATAC CCTGGTAGTC
  781 CGCACAGTAA ACGATGGATG CCCGCTGTTG GTACCTGGTR TCAGCGGCTA AGCGAAAGCA
  841 TTAAGCATCC CACCTGGGGA GTACGCCGGC AACGGTGAAA CTCAAAGGAA TTGACGGGGG
  901 CCCGCACAAG CGGAGGAACA TGTGGTTTAA TTCGATGATA CGCGAGGAAC CTTACCCGGG
  961 CTTGAATTGC AGAGGAAGGA TTTAGAGATA ATGACGCCCT TCGGGGTCTC TGTGAAGGTG
1021 CTGCATGGTT GTCGTCAGCT CGTGCCGTGA GGTGTCGGCT TAAGTGCCAT AACGAGCGCA
1081 ACCCCTCTCT TCAGTTGCCA TCAGGTTAAG CTGGGCACTC TGGAGACACT GCCACCGTAA
1141 GGTGTGAGGA AGGTGGGGAT GACGTCAAAT CAGCACGGCC CTTACGTCCG GGGCTACACA
1201 CGTGTTACAA TGGCCGGTAC AGAGGGACGG TGTAATGCAA ATTGCATCCA ATCTTGAAAG
1261 CCGGTCCCAG TTCGGACTGG GGTCTGCAAC CCGACCCCAC GAAGCTGGAT TCGCTAGTAA
1321 TCGCGCATCA GCCATGGCGC GGTGAATACG TTCCCGGGCC TTGTACACAC CGCCCGTCAA
1381 GCCATGAAAG CCGGGGGTGC CTGAAGTCCG TGACCGCAAG GATCGGCCTA GGGCAAAACT
1441 GGTGATTGGG GCTAAGTCGT AACAAGGTAG CCGTACCGGA AGGTGCGGCT GGAACACCTC
1501 CTTTCT
SEQ ID NO: 15 (Proteus mirabilis strain ATCC 29906 16S ribosomal RNA
gene, partial sequence)
NCBI Reference Sequence: NR_114419.1
      1 TGATCCTGGC TCAGATTGAA CGCTGGCGGC AGGCCTAACA CATGCAAGTC GAGCGGTAAC
    61 ACGAGAAAGC TTSCTTTCTT GCTCACGASC CGCGGACGCG TGAGTAATGT ATCGGGATCT
  121 GCCCGATAGA GGGGGATAAC TACTGGAAAC GGTGGCTAAT ACCGCATAAT GTCTACGGAC
  181 CAAAGCAGGG GCTCTTCGGA CCTTGCACTA TCGGATGAAC CCATATGGGA TTAGCTAGTA
  241 GGTGGGGTAA AGGCTCACCT AGGCGACGAT CTCTAGCTGG TCTGAGAGGA TGATCAGCCA
  301 CACTGGGACT GAGACACGGC CCAGACTCCT ACGGGAGGCA GCAGTGGGGA ATATTGCACA
  361 ATGGGCGCAA GCCTGATGCA GCCATGCCGC GTGTATGAAG AAGGCCTTAG GGITGTAAAG
  421 TACTTTCAGC GGGGAGGAAG GTGATAAGGT TAATACCCTT ATCAATTGAC GTTACCCGCA
  481 GAAGAAGCAC CGGCTAACTC CGTGCCAGCA GCCGCGGTAA TACGGAGGGT GCAAGCGTTA
  541 ATCGGAATTA CTGGGCGTAA AGCGCACGCA GGCGGTCAAT TAAGTCAGAT GTGAAAGCCC
  601 CGAGCTTAAC TTGGGAATTG CATCTGAAAC TGGTTGGCTA GAGTCTTGTA GAGGGGGGTA
  661 GAATTCCATG TGTAGCGGTG AAATGCGTAG AGATGTGGAG GAATACCGGT GGCGAAGGCG
  721 GCCCCCTGGA CAAAGACTGA CGCTCAGGTG CGAAAGCGTG GGGAGCAAAC AGGATTAGAT
  781 ACCCTGGTAG TCCACGCTGT AAACGATGTC GATTTAGAGG TTGTGGTCTT GAACCGTGGC
  841 TTCTGGAGCT AACGCGTTAA ATCGACCGCC TGGGGAGTAC GGCCGCAAGG TTAAAACTCA
  901 AATGAATTGA CGGGGGCCCG CACAAGCGGT GGAGCATGTG GTTTAATTCG ATGCAACGCG
  961 AAGAACCTTA CCTACTCTTG ACATCCAGCG AATCCTTTAG AGATAGAGGA GTGCCTTCGG
1021 GAACGCTGAG ACAGGTGCTG CATGGCTGTC GTCAGCTCGT GTTGTGAAAT GTTGGGTTAA
1081 GTCCCGCAAC GAGCGCAACC CTTATCCTTT GTTGCCAGCA CGTAATGGTG GGAACTCAAA
1141 GGAGACTGCC GGTGATAAAC CGGAGGAAGG TGGGGATGAC GTCAAGTCAT CATGGCCCTT
1201 ACGAGTAGGG CTACACACGT GCTACAATGG CAGATACAAA GAGAAGCGAC CTCGCGAGAG
1261 CAAGCGGAAC TCATAAAGTC TGTCGTAGTC CGGATTGGAG TCTGCAACTC GACTCCATGA
1321 AGTCGGAATC GCTAGTAATC GTAGATCAGA ATGCTACGGT GAATACGTTC CCGGGCCTTG
1381 TACACACCGC CCGTCACACC ATGGGAGTGG GTTGCAAAAG AAGTAGGTAG CTTAACCTTC
1441 GGGAGGGCGC TTACCACTTT GTGATTCATG ACTGGGGTGA AGTCGTAACA AGGTAGC
```

1.-46. (canceled)

47. A pharmaceutical composition comprising:
a. a preparation consisting of at least four species of isolated, viable, anaerobic gut bacteria selected from the group consisting of: Bacteroides fragilis, Bacteroides ovatus, Bacteroides vulgatus, Parabacteroides distasonis, and Prevotella melaninogenica, in an amount sufficient to treat or prevent a food allergy when administered to an individual in need thereof, and
b. a pharmaceutically acceptable carrier.

48.-67. (canceled)

68. The pharmaceutical composition of claim 47, wherein the pharmaceutical composition is formulated to deliver the viable bacteria to the small intestine.

69. The pharmaceutical composition of claim 47, wherein the pharmaceutical composition is formulated as a capsule, a gel, a pastille, or a tablet.

70. The pharmaceutical composition of claim 47, wherein the pharmaceutical composition comprises an enteric coating.

71. (canceled)

72. The pharmaceutical composition of claim 47, wherein the species of viable gut bacteria are present in substantially equal biomass.

73. The pharmaceutical composition of claim 47, wherein the pharmaceutical composition is formulated to deliver a dose of at least 1×10⁷ colony forming units (CFUs).

74.-77. (canceled)

78. The pharmaceutical composition of claim 70, wherein the enteric coating comprises a polymer, a nanoparticle, a fatty acid, a shellac, or a plant fiber.

79. (canceled)

80. The pharmaceutical composition of claim 47, wherein the pharmaceutical composition is encapsulated, is a lyophilisate, is formulated in a food item, or is formulated as a liquid, gel, fluid-gel, or nanoparticles in a liquid.

81. (canceled)

82. (canceled)

83. A method for the treatment, or prevention of food allergy, the method comprising: administering to a subject the pharmaceutical composition of claim 47, thereby treating or preventing the food allergy in the subject.

84. (canceled)

85. The method of claim 83, wherein the pharmaceutical composition is administered by oral administration, enema, suppository, or orogastric tube.

86. (canceled)

87. (canceled)

88. The pharmaceutical composition of claim 47, wherein the gut bacteria are formulated to maintain anaerobic conditions.

89. (canceled)

90. The method of claim 83, further comprising administering to the subject a prebiotic composition.

91. (canceled)

92. (canceled)

93. The method of claim 83, wherein the subject is a human subject.

94.-101. (canceled)

102. The method of claim 83, wherein the food allergy comprises allergy to one or more of soy, wheat, eggs, dairy, peanuts, tree nuts, shellfish, fish, mushrooms, stone fruit or other fruit.

103. The method of claim 83, wherein the pharmaceutical composition is administered before the set is first exposed to a potential food allergen.

104. (canceled)

105. The method of claim 83, wherein the subject has been diagnosed with a food allergy.

106. The method of claim 83, wherein the subject is pretreated with an antibiotic.

107. A method for reducing or eliminating a subject's immune reaction to a food allergen, the method comprising: administering to the subject the pharmaceutical composition of claim 47, thereby reducing or eliminating the subject's immune reaction to the food allergen.

108. (canceled)

109. The method of claim 107, wherein the food allergen is selected from the group consisting of: soy, wheat, eggs, dairy, peanuts, tree nuts, shellfish, fish, mushrooms, stone fruit and other fruit.

110. The method of claim 107, wherein the pharmaceutical composition is administered by oral administration, enema, suppository, or orogastric tube.

111. (canceled)

112. The method of claim 107, wherein the subject is a human subject.

113.-121. (canceled)

122. The method of claim 107, wherein the pharmaceutical composition is administered after the subject's initial exposure to a potential food allergen.

123. (canceled)

124. The method of claim 107, wherein the subject is pretreated with an antibiotic.

125. The method of claim 107, wherein the subject is pretreated with a fasting period not longer than 24 hours prior to the administration of the pharmaceutical composition.

126.-132. (canceled)

133. A method of reducing the number or activity of Tₕ2 cells in a tissue of an individual in need thereof, the method comprising administering the pharmaceutical composition of claim 47 to the individual.

134. The method of claim 133, wherein the tissue is a gut tissue.
